(19) United States
(12) Patent Application Publication
(10) Pub. No.: US 2023/0405025 A1
(43) Pub. Date: Dec. 21, 2023

(54) CANNABINOID-CONTAINING COMPOSITIONS AND USE FOR TREATING AND PREVENTING DISEASES

(71) Applicant: Max Biology Co. Ltd., Phnom Penh (KH)

(72) Inventors: Sunil SHAH, Birmingham (GB); Sean NGU, Phnom Penh (KH)

(21) Appl. No.: 18/030,439

(22) PCT Filed: Oct. 4, 2021
(86) PCT No.: PCT/IB2021/059094

§ 371 Date: Apr. 5, 2023

Related U.S. Application Data
(60) Provisional application No. 63/087,618, filed on Oct. 5, 2020.

(51) Int. Cl. A61K 31/00; A61K 31/575; A61K 31/618; A61K 31/355; A61K 45/06; A61K 9/00; A61K 31/01; A61K 31/015; A61K 31/352; A61K 9/16; A61P 17/00; A61P 27/02

(52) U.S. Cl. CPC A61K 31/658; A61K 31/575; A61K 31/618; A61K 31/355; A61K 45/06; A61K 9/0051; A61P 27/02; A61K 31/015; A61K 31/352; A61K 9/0014; A61K 9/1617; A61K 9/1641; A61P 17/00; A61K 31/01

(57) ABSTRACT

The invention provides compositions containing a cannabinoid (e.g., cannabidiol) and optionally one or more bioactive agents for the treatment and/or prevention of diseases including ophthalmic diseases or conditions, dermatologic conditions, infections, inflammatory diseases, and vision disorders. The invention also provides compositions and methods of preparing the compositions containing cannabinoids for cosmetic dermatologic applications and a pharmaceutical eluting contact lens. Furthermore, the invention provides delivery vehicles including lipid-polymer particles and methods of preparing the lipid-polymer particles to deliver the compositions to a subject.

### BACKGROUND OF THE INVENTION

Ophthalmic conditions such as dry eye disease affect millions of adults in the United States. Meibomian Gland Dysfunction (MGD) is a leading cause of dry eye disease and along with blepharitis, and meibomianitis, it is characterized by inflammation of meibomian glands and skin areas around the eyelids. Infections of the skin (e.g., Acne) and genitalia (e.g., bacterial vaginosis or candidiasis) are also commonly associated with inflammation. The present disclosure addresses the need for new pharmacological formulations that are effective for treating ophthalmic, dermatologic, infective, inflammatory diseases as well as vision disorders, such as presbyopia, myopia, and astigmatism, and also effective for cosmetic applications relating to skin pigmentation. Furthermore, delivery of bioactive agents with low solubility in aqueous media has been a challenge for drug delivery. An approach for targeted delivery of insoluble bioactive agents has been the use of liposomes and/or micellar nanoparticles as carrier systems. However, challenges with stability, degradation, and bioavailability have hindered the widespread implementation of such delivery systems. Accordingly, new formulations of delivery systems are required for effective delivery of bioactive agents.

### SUMMARY OF THE INVENTION

The invention provides compositions containing cannabinoids (e.g., cannabidiol) and optionally other bioactive agents for the treatment and/or prevention of diseases including ophthalmic diseases or conditions, dermatologic conditions, infections, inflammatory diseases, and vision disorders. The invention also provides compositions and methods of preparing the compositions containing cannabinoids for cosmetic dermatologic applications and a pharmaceutical eluting contact lens. Furthermore, the invention provides delivery vehicles including lipid-polymer particles and methods of preparing the lipid-polymer particles to deliver the compositions to a subject.

In one aspect, the invention features a pharmaceutical composition for the treatment or prevention of an ophthalmic condition, including therapeutically effective amounts of at least one cannabinoid (e.g., cannabidiol or derivatives thereof), and a pharmaceutically acceptable excipient or carrier, wherein the cannabinoid is present in a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition. In some embodiments, the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

In some embodiments, the composition further includes one or more terpenes selected from the group consisting of myrcene, beta-caryophyllene, linalool, alpha pinene, beta-pinene, ocimene, terpinolene, ocimene, terpinolene, alpha terpineol, alpha terpinene, gamma terpinene, alpha phellandrene, cymene, camphene, delta-3-carene, fenchol, 1,8-cineole, nerolidol, borneol, eucalyptol, camphene, and limonene, wherein one or more terpene is present at a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition.

In further embodiments, the composition includes one or more flavonoid selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, isoflavonoids, and a combination of one or more flavonoid, wherein one or more flavonoid is present at a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition.

In some embodiments, the composition includes β-sitosterol, wherein the β-sitosterol is present at a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition. In some embodiments, the composition further includes methyl salicylate, tocopherol, selected from the group consisting of alpha tocopherol, gamma tocopherol, and a combination of alpha and gamma tocopherol, alkaloids, lignans, or a combination of alkaloids and lignans.

In some embodiments, the composition includes a thickening agent, a solubilizing agent, a tonicity agent, a pH adjuster, a preservative, an antioxidant, osmotic agent, micellization agent, demulcent, surfactant, or a combination thereof. In some embodiments, the thickening agent is selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, and combinations thereof. In some embodiments, the solubilizing agent is selected from ethanol, glycerin, propylene glycol, polyethylene glycol, copolymers of ethylene oxide and propylene glycol, or combinations thereof. In some embodiments, the tonicity agent is selected from the group consisting of phosphate-buffered saline (PBS), Alsever's solution, Tris-buffered saline (TBS), water, balanced salt solutions (BSS), sodium chloride, potassium chloride, calcium chloride, magnesium chloride, mannitol, sorbitol, dextrose, glycerin, propylene glycol, ethanol, trehalose, or combinations thereof. In some embodiments, the preservative is selected from the group consisting of benzalkonium chloride (BAK), cetrimonium, sodium perborate, ethylenediaminetetraacetic acid (EDTA), chlorobutanol, or combinations thereof. In some embodiments, the antioxidant is selected from the group consisting of citric acid, sodium sulfite, vitamin E, carnosine, N-acetylcarnosine, pyruvate, resveratrol, astaxanthin, glutathione, cysteine, cysteine ascorbate, or combinations thereof.

In some embodiments, the pharmaceutical composition is encapsulated in liposomes. In some embodiments, the composition includes a liquid formulation suitable for use as an eye drop. In some embodiments, the composition includes an aqueous solution including at least one water soluble cannabinoid and saline. In some embodiments, the composition is coated on nanoparticles.

In some embodiments, the composition is formulated as a gel, a thin film, an ointment, non-aqueous solution, a solid form, a paste, a liquid, an aerosol, a mist, a polymer, a film, an emulsion, a suspension, or an injectable formulation. In some embodiments, the cannabinoid is present in the composition at a concentration of about 1% (w/w) to about 10% (w/w) (e.g., 2% (w/w), 3% (w/w), 4% (w/w), 5% (w/w), 6% (w/w), 7% (w/w), 8% (w/w), 9% (w/w), or 10% (w/w)) and suspended in a non-allergic pharmaceutically acceptable carrier comprising white petrolatum at a concentration of about 10% (w/w) to about 80% (w/w) (e.g., 20% (w/w), 30% (w/w), 40% (w/w), 50% (w/w), 60% (w/w), 70% (w/w), or 80% (w/w)) mineral oil at a concentration of about 10% (w/w) to about 50% (w/w) (e.g., about 20% (w/w), 30% (w/w), 40% (w/w), or 50% (w/w)) and lanolin at a concentration of about 1% (w/w) to about 5% (w/w) (e.g., 2% (w/w), 3% (w/w), 4% (w/w), or 5% (w/w)).

In some embodiments, the composition is coated on punctal plugs and in further embodiments the punctal plugs are coated or impregnated with the pharmaceutical composition, e.g., impregnated with nanoparticles containing the pharmaceutical composition.

In some embodiments, the pharmaceutical composition further includes micellar water. In further embodiments, the pharmaceutical composition includes micellar water and a cannabinoid. In some embodiments, the pharmaceutical composition includes micellar water, a cannabinoid, and a bioactive agent.

In another aspect, the invention provides a pharmaceutical eluting contact lens including a contact lens coated or embedded with the pharmaceutical composition described herein. In some embodiments, the pharmaceutical composition is released from the contact lens when the lens is placed on an individual's eye.

In another aspect, the invention provides a method of treating an ophthalmic condition of an individual including a) providing a pharmaceutical composition described herein and b) administering a therapeutically effective amount of the pharmaceutical composition to an eye of an individual suspected of having an ophthalmic condition. In some embodiments, the pharmaceutical composition is in the form of an eye drop, an ointment, a contact lens coated or embedded with the pharmaceutical composition, or a punctal plug coated with the pharmaceutical composition, and administering a therapeutically effective amount of the pharmaceutical composition includes applying the eye drop, the ointment, the contact lens, or the punctal plug to the eye of the individual.

In some embodiments, the ophthalmic condition is keratitis. In some embodiments, the keratitis is bacterial keratitis, protozoal keratitis, fungal keratitis, or viral keratitis.

In some embodiments, the ophthalmic condition is conjunctivitis. In some embodiments, the conjunctivitis is bacterial conjunctivitis or viral conjunctivitis.

In some embodiments, the ophthalmic condition is episcleritis, scleritis, corneal abrasion, inflammation of the eye, injury to the eye following eye surgery (e.g., laser eye surgery), blepharoconjunctivitis, ocular rosacea, or glaucoma.

In another aspect, the invention provides a method for treating a human suffering from dry eye including a) providing a pharmaceutical composition described herein and b) administering a therapeutically effective amount of the pharmaceutical composition to an eye of the human suffering from dry eye.

In another aspect, the invention provides a method for treating a human suffering from meibomianitis including a) providing a pharmaceutical composition described herein and b) administering a therapeutically effective amount of the pharmaceutical composition to an eye of the human suffering from meibomianitis.

In some embodiments, the pharmaceutical composition is a liquid ophthalmic composition, a solution, a suspension, an emulsion, or an in-situ gel system. In some embodiments, wherein the pharmaceutical composition is a solid ophthalmic composition selected from the group consisting of a gel and an ointment, the pharmaceutical composition is administered to an eyelid of the human.

In another aspect, the invention provides a method for treating a human suffering from blepharitis including a) providing a pharmaceutical composition described herein and b) administering a therapeutically effective amount of the pharmaceutical composition to an eye of the human suffering from blepharitis.

In some embodiments, the pharmaceutical composition is a liquid ophthalmic composition. In some embodiments, the liquid ophthalmic composition is selected from the group consisting of a solution, a suspension, an emulsion, and an in-situ gel system.

In some embodiments, the pharmaceutical composition is a solid ophthalmic composition that is a gel or an ointment, and the pharmaceutical composition is administered prior to sleep (e.g., at nighttime or daytime) to an eye of the human, e.g., for the treatment or dry eye or MGD).

In another aspect, the invention provides a kit for treating an ophthalmic condition including a) a first ophthalmic formulation containing a cannabinoid or derivatives thereof for administration during daytime and b) a second ophthalmic formulation containing a cannabinoid or derivatives thereof for administration prior to sleep (e.g., at nighttime or at daytime) wherein the second ophthalmic formulation has a higher viscosity than the first ophthalmic formulation. In some embodiments, the first and the second ophthalmic formulation contains a cannabinoid or derivative thereof in a concentration between about 0.01% to about 10% by weight (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%). In some embodiments, the first ophthalmic formulation is an eye drop, wherein the eye drop is a solution, a suspension, or an emulsion. In some embodiments, the second ophthalmic formulation is selected from the group consisting of an in-situ gel, a gel, and an ointment.

In some embodiments of any of the above aspects, the pharmaceutical composition further includes micellar water. In further embodiments, the pharmaceutical composition includes micellar water and a cannabinoid. In some embodiments, the pharmaceutical composition includes micellar water, a cannabinoid, and a bioactive agent.

In another aspect, the invention provides a method for treating an ophthalmic condition of an individual including a) administering a first ophthalmic formulation to an eye of the individual, wherein the first ophthalmic formulation is a liquid ophthalmic formulation that includes at least one cannabinoid or derivatives thereof in a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition, and a pharmaceutically acceptable excipient or carrier and b) administering a second ophthalmic formulation to an eye, wherein the second ophthalmic formulation includes at least one cannabinoid or derivatives thereof in a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition, and a pharmaceutically acceptable excipient or carrier, and wherein the second ophthalmic formulation has a higher viscosity than the first ophthalmic formulation. In some embodiments, the second ophthalmic formulation is administered to an area surrounding the eye. In some embodiments, the second ophthalmic formulation is administered to an area surrounding the eyelid. In some embodiments, the first ophthalmic formulation is selected from the group consisting of a solution, a suspension, and an emulsion. In some embodiments, the second ophthalmic formulation is selected from the group consisting of an in-situ forming gel, a gel, and an ointment. In some embodiments, the second ophthalmic formulation is administered at about bedtime. In some embodiments the first ophthalmic formulation is administered during daytime or during daylight hours. In some embodiments, the first ophthalmic formulation is administered at a dosing schedule selected from the group consisting of once per day, twice per day, three times per day, four times per day, five times per day, six times per day, seven times per day, eight times per day, nine times per day, ten times per day, eleven times per day, twelve times per day, and hourly.

In some embodiments the composition further includes Manuka honey.

In another aspect, the invention provides a pharmaceutical composition including therapeutically effective amounts of a) a cannabinoid and b) a parasympathetic agonist. In some embodiments, the pharmaceutical composition further includes an active agent selected from the group consisting of a sympathetic antagonist, a sympathetic agonist, and combinations thereof. In some embodiments, the active agent is a sympathetic antagonist. In some embodiments, the active agent is a combination of a sympathetic agonist and a sympathetic antagonist. In some embodiments, the sympathetic antagonist is an alpha-adrenergic blocking agent such as dapiprazole and thymoxamine. In some embodiments, the cannabinoid is cannabidiol. In some embodiments, the active agent is a sympathetic agonist. In some embodiments the cannabinoid is CBD-1, the parasympathetic agonist is pilocarpine, and the sympathetic agonist is brimonidine.

In some embodiments, the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

In some embodiments, the parasympathetic agonist is a cholinergic agonist. In further embodiments, the parasympathetic agonist is selected from the group consisting of bethanechol, carbamylcholine, cevimeline, and pilocarpine. In some embodiments, the parasympathetic agonist is a cholinesterase inhibitor. In further embodiments, the cholinesterase inhibitor is selected from the group consisting of delta-9-tetrahydrocannabinol, carbamates, physostigmine, neostigmine, pyridostigmine, ambenonium, demecarium, rivastigmine, phenanthrene derivatives, galantamine, caffeine—noncompetitive, piperidines, donepezil, tacrine, edrophonium, huperzine, ladostigil, ungeremine and lactucopicrin.

In some embodiments, the sympathetic agonist is selected from the group consisting of brimonidine, clonidine, guanfacine, guanabenz, guanoxabenz, guanethidine, iopidine, tizanidine, and xylazine.

In some embodiments, the sympathetic agonist is an adrenergic agonist. In further embodiments, the adrenergic agonist is a α2 adrenergic agonist.

In some embodiments, the pharmaceutical composition includes one or more terpenes selected from the group consisting of myrcene, beta-caryophyllene, linalool, alpha pinene, beta pinene, ocimene, terpinolene, ocimene, terpinolene, alpha terpineol, alpha terpinene, gamma terpinene, alpha phellandrene, cymene, camphene, delta-3-carene, fenchol, 1,8-cineole, nerolidol, borneol, eucalyptol, camphene, and limonene, wherein one or more terpene is present at a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition. In some embodiments, the pharmaceutical composition includes one or more flavonoid selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, isoflavonoids, and a combination of one or more flavonoid, wherein one or more flavonoid is present at a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition.

In some embodiments, the pharmaceutical composition includes a solubilizing agent is optionally selected from ethanol, glycerin, propylene glycol, polyethylene glycol, copolymers of ethylene oxide and propylene glycol, and combinations thereof.

In some embodiments, the pharmaceutical composition is formulated for application to the eye. In some embodiments, the pharmaceutical composition includes a liquid formulation suitable for use as an eye drop or a punctal plug.

In some embodiments, the composition includes CBD-1, pilocarpine or carbachol, and brimonidine or iopidine.

In another aspect, the invention provides a method of treating a vision disorder including a) providing a pharmaceutical composition described herein and b) administering a therapeutically effective amount of the pharmaceutical composition to an eye of a subject identified as having a vision disorder. In some embodiments, the pharmaceutical composition is in the form of an eye drop or an ointment.

In some embodiments, the vision disorder is selected from the group consisting of presbyopia, hypermetropia, and astigmatism. In some embodiments, the pharmaceutical composition is administered to the eye of the subject at least twice per day during a course of treatment. In some embodiments, the pharmaceutical composition is administered to the eye of the subject at least once per day during a course of treatment. In some embodiments, the pharmaceutical composition is administered to the eye of the subject at least once every two days during a course of treatment. In some embodiments, the pharmaceutical composition is administered to the eye of the subject at a dosing interval selected from the group consisting of four times a day, three times a day, twice a day, once a day, once every two days, once every three days, and once every week. In some embodiments, the pharmaceutical composition is administered each day for at least seven days.

In another aspect, the invention provides a topical composition for the treatment or prevention of a dermatological condition. The composition includes a therapeutically effective amount of at least one cannabidiol, its analogs, derivatives, or a combination thereof, wherein the cannabidiol, its analogs, derivatives, or a combination thereof is present in a concentration between about 0.001% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition, and appropriate topical pharmaceutically acceptable excipients or carriers.

In some embodiments, the therapeutically effective amount of the cannabidiol, its analogs, derivatives, or a combination thereof is about 0.01% to about 4% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 4%, e.g., about 2%, 3%, or 4%) by weight of the composition. In some embodiments, the therapeutically effective amount of the cannabidiol, its analogs, derivatives, or a combination thereof is about 0.1% to about 3% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 4%, e.g., about 2%, or 3%) by weight of the composition.

In some embodiments, the cannabidiol, its analogs, derivatives, or a combination thereof is one or more of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

In some embodiments, the topical composition includes one or more terpenes selected from the group consisting of myrcene, beta-caryophyllene, linalool, alpha pinene, beta-pinene, ocimene, terpinolene, ocimene, terpinolene, alpha terpinol, alpha terpinene, gamma terpinene, alpha phellandrene, cymene, camphene, delta-3-carene, fenchol, 1,8-cineole, nerolidol, borneol, eucalyptol, camphene, and limonene. In some embodiments, the one or more terpenes are present in a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition.

In some embodiments, the topical composition includes one or more flavonoid is selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, isoflavonoids, and a combination of one or more flavonoid, wherein one or more flavonoid is present in a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition.

In some embodiments, the topical composition includes β-sitosterol or methyl salicylate wherein β-sitosterol or methyl salicylate are present in a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition. In some embodiments, the topical composition further includes tocopherol, selected from the group consisting of alpha tocopherol, gamma tocopherol, and a combination of alpha and gamma tocopherol, wherein the tocopherol is present in a concentration between about 0.01% to about 20% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, 10%, 11%, 12%, 13%, 14%, 15%, 16%, 17%, 18%, 19%, or 20%) by weight of the composition.

In some embodiments, the topical composition further includes alkaloids, lignans, or a combination of alkaloids and lignans. In some embodiments, the topical composition further includes one or more active agents selected from the group consisting of an antibiotic, an antiseptic agent, antiprotozoal, an antifungal, an antibacterial agent, an analgesic and an antiviral agent.

In some embodiments, the topical composition includes a thickening agent, a solubilizing agent, a tonicity agent, a pH adjuster, a preservative, an antioxidant, osmotic agent, micellization agent, demulcent, surfactant, a co-surfactant, a penetration enhancer, a chelating agent, or a combination thereof. In some embodiments, the thickening agent is selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, cellulose, chitosan, and combinations thereof. In some embodiments, the solubilizing agent is selected from ethanol, glycerin, propylene glycol, polyethylene glycol, copolymers of ethylene oxide and propylene glycol, and combinations thereof. In some embodiments, the tonicity agent is selected from the group consisting of phosphate-buffered saline (PBS), Alsever's solution, Tris-buffered saline, water, balanced salt solutions (BSS), sodium chloride, potassium chloride, calcium chloride, magnesium chloride, mannitol, sorbitol, dextrose, glycerin, propylene glycol, ethanol, trehalose, and combinations thereof. In some embodiments, the preservative is selected from the group consisting of benzalkonium chloride (BAK), cetrimonium, sodium perborate, EDTA, chlorobutanol, and combinations thereof. In some embodiments, the antioxidant is selected from the group consisting of citric acid, sodium sulfite, vitamin E, carnosine, N-acetylcarnosine, pyruvate, resveratrol, astaxanthin, glutathione, cysteine, cysteine ascorbate, and combinations thereof.

In some embodiments, the topical composition includes a pharmaceutically acceptable carrier including a hydrophobic or lipophilic substance including a paraffin oil, mineral oil, petrolatum, bee wax, butylhydroxytoluene, liquid lanolin, propylene carbonate, an ester of a C8-C18 organic acid, a C8-C30 fatty alcohol, a silicone oil, a vegetable oil, a fractionated or hydrogenated vegetable oil, a monoglyceride, a diglyceride, a triglyceride, a phospholipid, dimethyl isosorbide, a volatile solvent, N-methylpyrrolidone, dimethylacetamide, dimethylformamide; dimethyl sulfoxide, or a combination thereof. In some embodiments, the composition is encapsulated in liposomes, micelles, noisome, fullerene, nanoshell, quantum dot, dendrimer, lipid-polymer nanoparticles or a combination thereof.

In some embodiments, the topical composition is coated on nanoparticles or charged polymers. In some embodiments, the composition is formulated as a foam, cream, paste, gel, aerosol, ointment, shampoo, or lotion.

In some embodiments, the topical composition further includes micellar water. In further embodiments, the topical composition includes micellar water and a cannabinoid. In some embodiments, the topical composition includes micellar water, a cannabinoid, and a bioactive agent.

In some embodiments, the cannabinoid is present in the composition at a concentration of about 1% (w/w) to about 10% (w/w) (e.g., 2% (w/w), 3% (w/w), 4% (w/w), 5% (w/w), 6% (w/w), 7% (w/w), 8% (w/w), 9% (w/w), or 10% (w/w)) and suspended in a non-allergic pharmaceutically acceptable carrier comprising white petrolatum at a concentration of about 10% (w/w) to about 80% (w/w) (e.g., 20% (w/w), 30% (w/w), 40% (w/w), 50% (w/w), 60% (w/w), 70% (w/w), or 80% (w/w)) mineral oil at a concentration of about 10% (w/w) to about 50% (w/w) (e.g., about 20% (w/w), 30% (w/w), 40% (w/w), or 50% (w/w)) and lanolin at a concentration of about 1% (w/w) to about 5% (w/w) (e.g., 2% (w/w), 3% (w/w), 4% (w/w), or 5% (w/w)).

In another aspect, the invention provides a method of treating a dermatological condition of a subject, including a) providing a topical composition of any one of claims and b) topically applying a therapeutically effective amount of the topical composition to the subject's skin suspected of having a dermatological condition. In some embodiments, the dermatological condition is eczema. In some embodiments, the dermatological condition is dermatitis. In some embodiments, the dermatitis is atopic dermatitis, contact dermatitis, seborrheic dermatitis, perioral dermatitis, or xerotic eczema. In some embodiments, the dermatological condition is psoriasis, acne vulgaris, dryness of the skin, tinea pedis, hives, impetigo, non-melanoma cancer, itching dermatosis, acne rosacea, aging of skin, or dandruff.

In another aspect, the invention provides a pharmaceutical composition for the treatment or prevention of bacterial vaginosis. The composition includes therapeutically effective amounts of at least one cannabinoid or derivatives thereof, and a pharmaceutically acceptable excipient or carrier, wherein the cannabinoid is present in a concentration between about 0.01% to about 30% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%) by weight of the composition. In some embodiments the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1). In some embodiments, the composition further includes a thickening agent, a solubilizing agent, a pH adjuster, a preservative, surfactant, or a combination thereof. In some embodiments, the thickening agent is selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, and combinations thereof.

In some embodiments the preservative is selected from the group consisting of benzalkonium chloride (BAK), cetrimonium, sodium perborate, EDTA, chlorobutanol, and combinations thereof.

In some embodiments, the composition is a cream, an ointment, a gel, a suppository, or a capsule. In some embodiments, the composition is non-flowing and the concentration of the cannabinoid is between about 0.1% and about 30% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%) (w/w).

In some embodiments, the composition includes an antibiotic wherein the antibiotic is selected from the group consisting of metronidazole, clindamycin, tinidazole, tetracycline, amoxicillin, ampicillin, lumefloxacin, norfloxacin, afloxam, ciproflaxin, azithromycin and cefltoxine. In some embodiments, the composition includes a steroid wherein the steroid is selected from the group consisting of hydrocortisone, cortisone acetate, prednisone, prednisolone, methylprednisolone, dexamethasone, betamethasone, triamcinolone, and beclomethasone, and fluorometholone.

In another aspect, the invention provides a method of treating an individual having bacterial vaginosis, including a) providing a pharmaceutical composition including a therapeutically effective amount of at least one cannabinoid or derivatives thereof and pharmaceutically acceptable excipients and b) administering a therapeutically effective amount of the pharmaceutical composition to a vulvovaginal surface of the individual.

In some embodiments, the pharmaceutical composition is in a form selected from the group consisting of a cream, an ointment, a gel, a suppository, and a capsule. In further embodiments, administering a therapeutically effective amount of the pharmaceutical composition to the vulvovaginal surface includes applying the ointment, gel, or cream to the mucosal surfaces in the vaginal cavity of the individual. In some embodiments, administering a therapeutically effective amount of the pharmaceutical composition to the vulvovaginal surface includes applying the ointment, gel, or cream to a non-mucosal surface of a vulva of the individual. In some embodiments, the pharmaceutical composition is in the form of a suppository or capsule and administering a therapeutically effective amount of the pharmaceutical composition to the vulvovaginal surface includes inserting the suppository or capsule into the vaginal cavity of the individual.

In some embodiments, the pharmaceutical composition further includes an antibiotic. In further embodiments, the antibiotic is selected from the group consisting of metronidazole, clindamycin, tinidazole, tetracycline, amoxicillin, ampicillin, lumefloxacin, norfloxacin, afloxam, ciproflaxin, azithromycin and cefltoxine.

In some embodiments, the pharmaceutical composition further includes micellar water. In further embodiments, the pharmaceutical composition includes micellar water and a cannabinoid. In some embodiments, the pharmaceutical composition includes micellar water, a cannabinoid, and a bioactive agent.

In another aspect, the invention provides a method of treating an individual having candidiasis, including a) providing a pharmaceutical composition that includes a therapeutically effective amount of at least one cannabinoid or derivatives thereof and pharmaceutically acceptable excipients and b) administering a therapeutically effective amount of the pharmaceutical composition to a vulvovaginal surface of the individual.

In some embodiments, the pharmaceutical composition is in a form selected from the group consisting of a cream, an ointment, a gel, a suppository, and a capsule. In some embodiments, the pharmaceutical composition is in the form of an ointment, gel, or cream, and wherein administering a therapeutically effective amount of the pharmaceutical composition to the vulvovaginal surface includes applying the ointment, gel, or cream to the mucosal surfaces in the vaginal cavity of the individual. In some embodiments, the pharmaceutical composition is in the form of an ointment, gel, or cream, and wherein administering a therapeutically effective amount of the pharmaceutical composition to the vulvovaginal surface includes applying the ointment, gel, or cream to a non-mucosal surface of a vulva of the individual. In some embodiments, the pharmaceutical composition is in the form of a suppository or capsule, and wherein administering a therapeutically effective amount of the pharmaceutical composition to the vulvovaginal surface includes inserting the suppository or capsule into the vaginal cavity of the individual. In some embodiments, the pharmaceutical composition further includes an antifungal, e.g., fluconazole. In some embodiments, the pharmaceutical composition further includes a topical steroid. In some embodiments, the pharmaceutical composition further includes an antifungal (e.g., fluconazole) and a topical steroid. In some embodiments, the pharmaceutical composition further includes an antibiotic. In some embodiments, the antibiotic is selected from the group consisting of metronidazole, clindamycin, tinidazole, tetracycline, amoxicillin, ampicillin, lumefloxacin, norfloxacin, afloxam, ciproflaxin, azithromycin and cefltoxine.

In some embodiments, the pharmaceutical composition further includes micellar water. In further embodiments, the pharmaceutical composition includes micellar water and a cannabinoid. In some embodiments, the pharmaceutical composition includes micellar water, a cannabinoid, and a bioactive agent.

In another aspect, the invention provides a kit for treating bacterial vaginosis or candidiasis, including a) a pessary containing or coated with a pharmaceutical composition that includes a cannabinoid or derivatives thereof and b) a topical formulation including a cannabinoid or a derivative thereof.

In another aspect, the invention provides a topical composition for lightening skin that includes a therapeutically effective amount of a skin lightening agent and a therapeutically effective amount of at least one cannabinoid, its analogs, derivatives, or a combination thereof, wherein the cannabinoid, its analogs, derivatives, or a combination thereof is present in a concentration between about 0.001% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition, and appropriate topical pharmaceutically acceptable excipients or carriers.

In some embodiments, the therapeutically effective amount of the cannabinoid, its analogs, derivatives, or a combination thereof is about 0.01% to about 4% by weight of the composition. In some embodiments, the therapeutically effective amount of the cannabinoid, its analogs, derivatives, or a combination thereof is about 0.1% to about 3% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, or 3%) by weight of the composition.

In some embodiments, the cannabinoid, its analogs, derivatives, or a combination thereof is one or more of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

In some embodiments, the skin lightening agent is selected from the group consisting of hydroquinone, L-ascorbic acid, glycolic acid, lactic acid, arbutin, kojic acid, daisy flower extract, licorice extract, and placenta extract. In some embodiments, the skin lightening agent is an inhibitor of tyrosinase. In some embodiments, the skin lightening agent is hydroquinone. In some embodiments, the therapeutically effective amount of the skin lightening agent is about 4%. In some embodiments, the therapeutically effective amount of the skin lightening agent is about 10%.

In some embodiments, the topical composition further includes an antibiotic. In some embodiments, the antibiotic is selected from the group consisting of erythromycin, clindamycin, lymecycline, tinidazole, tetracycline, amoxicillin, ampicillin, lumefloxacin, norfloxacin, afloxam, ciproflaxin, azithromycin, and cefltoxine.

In some embodiments, the topical composition includes a steroid. In some embodiments, the steroid is selected from the group consisting of hydrocortisone, cortisone acetate, prednisone, prednisolone, methylprednisolone, dexamethasone, betamethasone, triamcinolone, and beclomethasone, fluorometholone. Other glucocorticoids useful in the treatment method include, but are not limited to, 21-acetoxypregnenolone, alelometasone, algestone, amcinonide, budesonide, chloroprednisone, clobetasol, clobetasone (EUMOVATE®), clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, tixocortol, triamcinolone acetonide, triamcinolone benetonide, triamcinolone hexacetonide, combinations thereof, and mixtures thereof.

In some embodiments, the topical composition includes a vitamin. In some embodiments, the vitamin is selected from the group consisting of vitamin A, vitamin B, vitamin C, vitamin D, and vitamin E.

In some embodiments, the topical composition includes an agent for treating acne. In some embodiments is selected from the group consisting of salicylic acid, glycolic acid, or lactic acid.

In some embodiments, the topical composition includes an agent for treating wrinkles and/or fine lines. In some embodiments the agent for treating wrinkles and/or fine lines is a retinoid. In some embodiments, the topical composition further includes one or more terpenes selected from the group consisting of myrcene, beta-caryophyllene, linalool, alpha pinene, beta-pinene, ocimene, terpinolene, ocimene, terpinolene, alpha terpinol, alpha terpinene, gamma terpinene, alpha phellandrene, cymene, camphene, delta-3-carene, fenchol, 1,8-cineole, nerolidol, borneol, eucalyptol, camphene, and limonene. In some embodiments, the topical composition includes β-sitosterol, wherein the β-sitosterol is present in a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition. In some embodiments, the topical composition includes methyl salicylate, wherein the methyl salicylate is present in a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition. In some embodiments, the topical composition includes tocopherol, selected from the group consisting of alpha tocopherol, gamma tocopherol, and a combination of alpha and gamma tocopherol, wherein the tocopherol is present in a concentration between about 0.01% to about 20% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, 10%, 11%, 12%, 13%, 14%, 15%, 16%, 17%, 18%, 19%, or 20%) by weight of the composition. In some embodiments, the topical composition includes alkaloids, lignans, or a combination of alkaloids and lignans.

In some embodiments, the topical composition includes one or more active agents selected from the group consisting of an antibiotic, an antiseptic agent, antiprotozoal, an antifungal, an antibacterial agent, an analgesic, and an antiviral agent.

In some embodiments, the topical composition includes a thickening agent, a solubilizing agent, a tonicity agent, a pH adjuster, a preservative, an antioxidant, osmotic agent, micellization agent, demulcent, surfactant, a co-surfactant, a penetration enhancer, a chelating agent, or a combination thereof. In some embodiments, the thickening agent is selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, cellulose, chitosan, and combinations thereof.

In some embodiments, the solubilizing agent is selected from ethanol, glycerin, propylene glycol, polyethylene glycol, copolymers of ethylene oxide and propylene glycol, and combinations thereof. In some embodiments, the tonicity agent is selected from the group consisting of phosphate-buffered saline (PBS), Alsever's solution, Tris-buffered saline, water, balanced salt solutions (BSS), sodium chloride, potassium chloride, calcium chloride, magnesium chloride, mannitol, sorbitol, dextrose, glycerin, propylene glycol, ethanol, trehalose, and combinations thereof. In some embodiments, the preservative is selected from the group consisting of benzalkonium chloride (BAK), cetrimonium, sodium perborate, EDTA, chlorobutanol, and combinations thereof. In some embodiments, the antioxidant is selected from the group consisting of citric acid, sodium sulfite, vitamin E, carnosine, N-acetylcarnosine, pyruvate, resveratrol, astaxanthin, glutathione, cysteine, cysteine ascorbate, and combinations thereof. In some embodiments, the topical composition includes a pharmaceutically acceptable carrier that includes a hydrophobic or lipophilic substance including a paraffin oil, mineral oil, petrolatum, bee wax, butylhydroxytoluene, liquid lanolin, propylene carbonate, an ester of a C8-C18 organic acid, a C8-C30 fatty alcohol, a silicone oil, a vegetable oil, a fractionated or hydrogenated vegetable oil, a monoglyceride, a diglyceride, a triglyceride, a phospholipid, dimethyl isosorbide, a volatile solvent, N-methylpyrrolidone, dimethylacetamide, dimethylformamide; dimethyl sulfoxide, or a combination thereof.

In some embodiments, the topical composition further includes micellar water. In further embodiments, the topical composition includes micellar water and a cannabinoid. In some embodiments, the topical composition includes micellar water, a cannabinoid, and a bioactive agent.

In some embodiments, the composition is encapsulated in liposomes, micelles, noisome, fullerene, nanoshell, quantum dot, dendrimer, lipid-polymer compositions (e.g., lipid polymer nanoparticles), or any combination thereof. In some embodiments, the composition is coated on nanoparticles or charged polymers. In some embodiments, the composition is formulated as a foam, cream, paste, gel, aerosol, ointment, shampoo, or lotion. In some embodiments, the cannabinoid is present in the composition at a concentration of about 1% (w/w) to about 10% (w/w) (e.g., 2% (w/w), 3% (w/w), 4% (w/w), 5% (w/w), 6% (w/w), 7% (w/w), 8% (w/w), 9% (w/w), or 10% (w/w)) and suspended in a non-allergic pharmaceutically acceptable carrier comprising white petrolatum at a concentration of about 10% (w/w) to about 80% (w/w) (e.g., 20% (w/w), 30% (w/w), 40% (w/w), 50% (w/w), 60% (w/w), 70% (w/w), or 80% (w/w)) mineral oil at a concentration of about 10% (w/w) to about 50% (w/w) (e.g., about 20% (w/w), 30% (w/w), 40% (w/w), or 50% (w/w)) and lanolin at a concentration of about 1% (w/w) to about 5% (w/w) (e.g., 2% (w/w), 3% (w/w), 4% (w/w), or 5% (w/w)).

In another aspect, the invention provides a method of treating a dermatological condition of a subject including a) providing a topical composition described herein and b) topically applying a therapeutically effective amount of the topical composition to the subject's skin suspected of having a dermatological condition.

In some embodiments, the dermatological condition is excessive darkening of the subject's skin, a liver spot, hyperpigmentation, associated with overexposure to ultraviolet radiation, age spot, or seborrheic keratosis.

In another aspect, the invention provides a method of lightening and/or whitening the skin color of a subject, including a) providing a topical composition described herein and b) topically applying a therapeutically effective amount of the topical composition to the subject's skin.

In another aspect, the invention provides a topical composition for treating acne. The composition includes a therapeutically effective amount of an acne agent and a therapeutically effective amount of at least one cannabinoid, its analogs, derivatives, or a combination thereof, wherein the cannabinoid, its analogs, derivatives, or a combination thereof is present in a concentration between about 0.001% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition, and appropriate topical pharmaceutically acceptable excipients or carriers.

In some embodiments, the therapeutically effective amount of the cannabinoid, its analogs, derivatives, or a combination thereof is about 0.01% to about 4% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, or 4%) by weight of the composition. In some embodiments, the therapeutically effective amount of the cannabinoid, its analogs, derivatives, or a combination thereof is about 0.1% to about 3% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, or 3%) by weight of the composition. In some embodiments, the cannabinoid, its analogs, derivatives, or a combination thereof is one or more of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1). In some embodiments, the acne treatment agent is selected from the group consisting of salicylic acid, glycolic acid, or lactic acid. In some embodiments, the acne treatment agent is a retinol. In some embodiments, the topical composition for treating acne further includes an antibiotic. In some embodiments, the antibiotic is selected from the group consisting of erythromycin, clindamycin, lymecycline, tinidazole, tetracycline, amoxicillin, ampicillin, lumefloxacin, norfloxacin, afloxam, ciproflaxin, azithromycin, and cefltoxine.

In some embodiments, the topical composition for treating acne further includes a steroid. In further embodiments, the steroid is selected from the group consisting of hydrocortisone, cortisone acetate, prednisone, prednisolone, methylprednisolone, dexamethasone, betamethasone, triamcinolone, and beclomethasone, fluorometholone. Other glucocorticoids useful in the treatment method include, but are not limited to, 21-acetoxypregnenolone, alelometasone, algestone, amcinonide, budesonide, chloroprednisone, clobetasol, clobetasone (EUMOVATE®), clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, tixocortol, triamcinolone acetonide, triamcinolone benetonide, triamcinolone hexacetonide, combinations thereof, and mixtures thereof.

In some embodiments, the topical composition for treating acne further includes a vitamin. In further embodiments, the vitamin is selected from the group consisting of vitamin A, vitamin B, vitamin C, vitamin D, and vitamin E.

In some embodiments, the topical composition further includes micellar water. In further embodiments, the topical composition includes micellar water and a cannabinoid. In some embodiments, the topical composition includes micellar water, a cannabinoid, and a bioactive agent.

In another aspect, the invention provides a method of treating acne of a subject, including a) providing a topical composition described herein and b) topically applying a therapeutically effective amount of the topical composition to the subject's skin suspected of having a dermatological condition.

In another aspect, the invention provides a composition that includes a therapeutically effective amount of a cannabinoid, wherein the pharmaceutical composition is a powder that includes a guest particle adhered to a carrier particle. In some embodiments, the guest particle, carrier particle, or both include at least one cannabinoid. In some embodiments the cannabinoid is selected from the group consisting of cannabigerol-type, cannabichromene-type, a cannabidiol-type, a cannabinol-type, a cannabielsoin-type, and iso-tetrahydrocannabinol-type, a cannabicyclol-type, and a cannabicitran-type cannabinoid. In some embodiments, the cannabinoid is a cannabidiol-type cannabinoid. In some embodiments, the cannabidiol-type cannabinoid is selected from the group consisting of cannabidiol (CBD-1), cannabidiolic acid (CBDA), cannabidiol monomethyl ether (CBDM), cannabidiol-C4 (CBD-C4), cannabidivarinic acid (CBDVA), cannabidivarin (CBDV), and cannabidiorcol (CBD-C1).

In some embodiments, the guest particle, carrier particle, or both further include a bioactive agent. In some embodiments, the bioactive agent is a sedative, antianxiety agent, or a combination thereof. In some embodiments, the bioactive agent is selected from the group consisting of pentobarbital, secobarbital, diazepam, chlordiazepoxide, prazepam, clonazepam, midazolam, nitrazepam oxazepam, lorazepam, alprazolam, buspirone, flurazepam, temazepam, triazolam, chloral hydrate, zolpidem, zopiclone, eszopiclone, and diphenhydramine.

In some embodiments, the carrier particle, the guest particle, or both may include a solubility controlling agent, wherein the solubility controlling agent includes an anionic surfactant, a cationic surfactant; a non-ionic surfactant, a zwitterionic surfactant, an amino acid, a sugar, a water-soluble polymer, a disintegrant, or a combination thereof.

In some embodiments, the carrier particle, the guest particle, or both includes a terpene selected from the group consisting of myrcene, beta-caryophyllene, linalool, alpha-pinene, beta-pinene, ocimene, terpinolene, ocimene, terpinolene, alpha-terpineol, alpha-terpinene, gamma terpinene, alpha phellandrene, cymene, camphene, delta-3-carene, fenchol, 1,8-cineole, nerolidol, borneol, eucalyptol, camphene, and limonene, wherein the terpene is present at a concentration between about 0.01% to about 10% (e.g., about 0.01% to about 0.1%, e.g., about 0.02%, 0.03%, 0.04%, 0.05%, 0.06%, 0.07%, 0.08%, 0.09%, or 0.1%, e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%) by weight of the composition.

In some embodiments, the guest particles are micronized particles, aggregated liposomes, or aggregated nanoparticles. In some embodiments, the guest particle includes at least one cannabidiol.

In some embodiments, the carrier particle includes lactose, D-mannitol, sorbitol, erythritol, α-trehalose dihydrate, dextrose, glucose monohydrate, maltitol, maltose, xylitol hydroxyapatite, D-raffinose anhydrous, raffinose pentahydrate, a surfactant, polyvinyl alcohol, polyvinylpyrrolidone, poly (lactic-co-glycolic acid) (PLGA)), microcrystalline cellulose (MCC), hydroxyl propyl methyl cellulose (HPMC), an amino acid, magnesium stearate or cyclodextrins.

In some embodiments, the pharmaceutical composition includes two guest particle types adhered to one or more carrier particle types, wherein a first guest particle includes a cannabinoid, and a second guest particle includes a bioactive agent. In some embodiments, the pharmaceutical composition includes two or more carrier particle types adhered to one or more guest particle types. In some embodiments, the guest particle includes at least one cannabinoid. In some embodiments, the guest particles are slowly released into a subject's lung when the pharmaceutical composition is inhaled by the individual. In some embodiments, the carrier particle size is at least 5 times the average particle size of the guest particles. In some embodiments, the pharmaceutical composition is formulated as a dry powder inhaler.

In another aspect, the invention provides a method of treating a condition of a subject, including a) providing a composition as described herein and b) administering a therapeutically effective amount of the composition to the respiratory tract of the individual identified as having the condition; wherein the condition is selected from the group consisting of a sleep disorder, anxiety, post-traumatic stress disorder, a psychosomatic condition, a painful condition, an inflammatory condition, asthma, and diabetes. In some embodiments, the composition is administered by insufflation. In some embodiments, the condition is a sleep disorder. In some embodiments, the condition is anxiety. In some embodiments, the condition is post-traumatic stress disorder. In some embodiments, the condition is a psychosomatic condition. In some embodiments, the condition is pain. In some embodiments, the condition is an inflammatory condition. In some embodiments, the condition is asthma. In some embodiments, the condition is diabetes. In some embodiments, the composition is administered to the nasal sinuses and/or the lungs. In some embodiments the psychosomatic condition is insomnia.

In another aspect, the invention provides a composition that includes a plurality of lipid-polymer composite particles encapsulating a bioactive agent. The lipid-polymer composite particles may include a block copolymer, a lipid (e.g., a neutral lipid, a cationic lipid, or an anionic lipid), and a sterol. The plurality of lipid-polymer composite particles may have a mean particle size of from about 10 nm to about 1000 nm (e.g., from about 10 nm to about 500 nm, from about 10 nm to about 100 nm, from about 500 nm to about 1000 nm).

In some embodiments, the bioactive agent is a therapeutic agent, a nutraceutical agent, or a recreational agent.

In some embodiments, the bioactive agent is a cannabinoid or a cannabinoid derivative, a terpene, a flavonoid, an antibiotic, an antiseptic agent, an antifungal, an antibacterial, an analgesic, a non-steroidal anti-inflammatory agent, an antiprotozoal agent, a steroid, an antiviral agent, a lipophilic drug, an anti-VEGF agent, an anti-glaucoma agent, nicotine or a nicotine analogue, cyclosporin A, tacrolimus, isotretinoin, propofol, griseofulvin or any combination thereof.

In some embodiments, the block copolymer is a poloxamer (e.g., poloxamer 407).

In some embodiments, the weight ratio of the poloxamer and the bioactive agent is from about 2 to about 15.

In some embodiments, the lipid includes a carbon chain of length from 4 to 22 and a head group having a neutral, or cationic, or anionic head group. In some embodiments, the lipid is a phosphatidylcholine, a phosphatidylserine, a phosphatidylglycerol, a phosphatidylethanolamine, or a phosphatidylinositol.

In some embodiments, the concentration of the lipid is from about 0.1 mol % to about 10 mol % (e.g., about 0.1% to about 1%, e.g., about 0.2%, 0.3%, 0.4%, 0.5%, 0.6%, 0.7%, 0.8%, 0.9%, or 1%, e.g., about 1% to about 10%, e.g., about 2%, 3%, 4%, 5%, 6%, 7%, 8%, 9%, or 10%).

In some embodiments, the sterol is a phytosterol, a synthetic sterol, cholesterol, or a cholesterol analog.

In some embodiments, the concentration of the sterol is form about 5 mol % to about 50 mol % (e.g., about 5 mol % to about 10% mol, e.g., about 6% mol, 7% mol, 8% mol, 9% mol, or 10% mol, e.g., about 10% mol to about 50% mol, e.g., 15% mol, 20% mol, 25% mol, 30% mol, 35% mol, 40% mol, 45% mol, of 50% mol) of the total lipid composition.

In some embodiments, the weight ratio of the sterol to the lipid is from about 0.01 to about 0.50 (e.g., about 0.01 to about 0.1, e.g., about 0.02, 0.03, 0.04, 0.05, 0.06, 0.07, 0.08, 0.09, or 0.1, e.g., about 0.1 to about 0.50, e.g., 0.2, 0.3, 0.4, or 0.5).

In another aspect, the invention features a method of providing a bioactive agent to a subject by administering the composition of any of the above embodiments to the subject.

In some embodiments, the bioactive agent includes a cannabinoid and the dose is from about 0.01 mg/kg to about 30 mg/kg (e.g., about 0.01 mg/kg to about 0.1 mg/kg, e.g., 0.02 mg/kg, 0.03 mg/kg, 0.04 mg/kg, 0.05 mg/kg, 0.06 mg/kg, 0.07 mg/kg, 0.08 mg/kg, 0.09 mg/kg, or 0.10 mg/kg, e.g., 0.1 mg/kg to about 1.0 mg/kg, e.g., 0.2 mg/kg, 0.3 mg/kg, 0.4 mg/kg, 0.5 mg/kg, 0.6 mg/kg, 0.7 mg/kg, 0.8 mg/kg, 0.9 mg/kg, or 1.0 mg/kg, e.g., about 1.0 mg/kg to about 10 mg/kg, e.g., 2.0 mg/kg, 3.0 mg/kg, 4.0 mg/kg, 5.0 mg/kg, 6.0 mg/kg, 7.0 mg/kg, 8.0 mg/kg, 9.0 mg/kg, or 10 mg/kg, e.g., about 10 mg/kg to about 30 mg/kg, e.g., 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, or 30 mg/kg).

In some embodiments, mode of administration is topical, orally, by injection, sublingually, buccally, rectally, vaginally, by ocular route, by otic route, by nasal route, by inhalation, by nebulization, or transdermally.

In some embodiments, the composition further includes micellar water. In further embodiments, the composition includes micellar water and a cannabinoid. In some embodiments, the composition includes micellar water, a cannabinoid, and a bioactive agent. In some embodiments the composition further includes an antioxidant (e.g., citric acid or sodium sulfite). In some embodiments the composition is prepared by incorporating a lipid via ethanol injection. In some embodiments the lipid is incorporated at a temperature between 25-75° C. (e.g., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 60° C., 61° C., 62° C., 63° C., 64° C., 65° C., 66° C., 67° C., 68° C., 69° C., 70° C., 71° C., 72° C., 73° C., 74° C., or 75° C.).

In another aspect, the invention features a method of preparing the composition of any of the above embodiments. In some embodiments, preparation of the composition of any of the above embodiments includes a multi-step process. In some embodiments, the multi-step process includes a first step including homogenization of the bioactive agent with the polymer of any of the above embodiments to produce a homogenized solution and a second step including injection (e.g., immersed injection) of the lipid and the sterol of any of the above embodiments into the homogenized solution of the first step.

In another aspect, the invention provides a composition including a therapeutically effective amount of a cannabinoid and micellar water. In some embodiments, the composition further includes a therapeutically effective amount of a bioactive agent. In some embodiments, the bioactive agent includes an antibiotic, an antifungal, an antiviral, an analgesic, an antiseptic, a steroid, a non-steroidal anti-inflammatory drug. In some embodiments, the cannabinoid is present in a concentration between 0.01% to about 10% by weight of the composition. In some embodiments, the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

In another aspect, the invention provides a method of treating a dermatological condition of a subject, including a) providing a composition herein described and b) administering a therapeutically effective amount of the composition to the skin of the subject identified as having the condition. In some embodiments, the condition is selected from the group consisting of acne, eczema, dermatitis, psoriasis, acne vulgaris, dryness of the skin, tinea pedis, hives, impetigo, non-melanoma cancer, itching dermatosis, acne rosacea, aging of skin, or dandruff.

In another aspect, the invention provides a method of treating an ophthalmic condition of a subject, including a) providing a composition herein described and b) administering a therapeutically effective amount of the composition to the eye of the subject identified as having the condition. In some embodiments, the condition is selected from the group consisting of dry eye, blepharitis, meibomianitis, keratitis, bacterial keratitis, protozoal keratitis, fungal keratitis, viral keratitis, conjunctivitis, bacterial conjunctivitis, viral conjunctivitis, episcleritis, scleritis, corneal abrasion, inflammation of the eye, injury to the eye following eye surgery, blepharoconjunctivitis, ocular rosacea, glaucoma, and contact lens intolerance.

### Definitions

To facilitate the understanding of this invention, a number of terms are defined below. Terms defined herein have meanings as commonly understood by a person of ordinary skill in the areas relevant to the invention. Terms such as “a,” “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, any values provided in a range of values include both the upper and lower bounds, and any values contained within the upper and lower bounds.

As used herein, term “about,” as used herein, refers to ±10% of a recited value.

The term “administer” means to introduce a formulation of the present invention into the body of a patient in need thereof to treat a disease or condition.

The term “bioactive agent” as used herein refers to any synthetic or naturally occurring compound (in free form, salt form or solvated or hydrated form) having a desired biological or physiological effect (e.g., therapeutic, diagnostic, or prophylactic effect), such as a protein, drug, antigen, nutrient, cosmetic, fragrance, flavoring, diagnostic, pharmaceutical, vitamin, or dietary agent and will be formulated at a level sufficient to provide an in vivo concentration at a functional level (including local concentrations for topical compositions). Under some circumstances one or more of components of the lipid matrix i) (e.g., components (a), (b), (c) and/or (d)) may also be an active agent, although it is preferred that the optional bioactive agent (iii) should not be one of these components (e.g., should not be a component of the lipid matrix). Most preferred active agents are pharmaceutical agents (e.g., APIs) including drugs, vaccines, and diagnostic agents.

The term “block copolymer” as used herein, refers to a linear polymer having regions or blocks along its backbone chain which are characterized by similar hydrophilicity, hydrophobicity, or chemistry.

The term “diblock copolymer” means a block copolymer comprising two blocks.

The term “triblock copolymer” means a block copolymer comprising three blocks.

The term “multiblock copolymer” means a block copolymer comprising a plurality of blocks.

As used herein, the term “encapsulate,” “encapsulated,” or “encapsulating” refers to an enclosure of a moiety (e.g., a bioactive agent as defined herein) within an enclosed polymer assembly structure, e.g., a micelle. An encapsulated bioactive agent (e.g., an encapsulated cannabinoid) is enclosed by the polymer assembly structure, e.g., such an encapsulated moiety is located in the hydrophobic interior of the polymer assembly structure (e.g., the lumen of a micelle).

The term “anionic head group,” as used herein, refers to a lipid head group that carries a net negative charge at physiological pH.

The terms “cannabidiol” and “CBD,” as used herein, refer to both the class of compounds and its prototypical member, cannabidiol. For the avoidance of confusion, the terms “cannabidiol,” “CBD,” and “cannabidiol derivative,” as used herein, refer the class of naturally occurring and synthetic phyto-cannabinoids based on the prototypical compound also commonly known as cannabidiol. The term “CBD-1” refers to the prototypical member of the CBD class, cannabidiol, which is named according to the IUPAC convention as 2-[(1R,6R)-6-Isopropenyl-3-methylcyclohex-2-en-1-yl]-5-pentylbenzene-1,3-diol (FIG. 1).

The terms “composition” or “pharmaceutical composition,” as used interchangeably herein, refers to a mixture comprising at least one pharmaceutically acceptable active ingredient, in combination with suitable pharmaceutically acceptable excipients.

The term “cationic head group,” as used herein, refers to a lipid head group that carries a net positive charge at physiological pH.

As used herein, the term “dry” means that a composition has a very low moisture content such that the particles are easy to disperse in an inhaler, to form an aerosol. This moisture content is generally below about 10% of the weight of the composition, usually below about 5% of the weight of the composition, and preferably below about 3% of the weight of the composition.

The term “neutral head group,” as used herein, refers to a lipid head group that exists in an uncharged form at physiological pH.

As used herein, “lipid nanoparticle” or “LNP” is a vesicle that includes a lipid layer encapsulating a substantially solid lipid core; the lipid core can contain a pharmaceutically active molecule. LNPs typically contain a cationic lipid, a non-cationic lipid, and a lipid that prevents aggregation of the particle (e.g., a PEG-lipid conjugate).

As used herein, the term “lipid-polymer composite particle” refers to a complex of molecules held together by noncovalent bonds, such as hydrogen bonds, Van der Waals forces, electrostatic interactions, hydrophobic effect, and Pi-Pi interactions. Lipid-polymer composite particles may include large complexes of molecules that form, e.g., sphere-like structures. Lipid-polymer composite particles include, for example, lipid nanoparticles and micelles.

As used herein, the term “micelle,” “micellar” or variants thereof, refers to a polymer assembly having a hydrophilic shell (or corona) and a hydrophobic and/or ionic interior. In addition, the term micelle may refer to any poly ion complex assembly consisting of a multiblock copolymer possessing a net positive charge and a suitable negatively charged polynucleotide.

The term “nanoparticle,” as used herein, refers to a polymer-based particle having a diameter in the nanometer range (e.g., 1 nm-1000 nm).

The term “nutraceutical agent” refers to any substance that is a food or a part thereof, and/or conferring extra health benefits in addition to the basic nutritional value found in foods. For example, nutraceutical agents may contain components from food sources. Exemplary nutraceutical agents include, but are not limited to, antioxidants, dietary supplements, fortified dairy products, plant extracts, vitamins, minerals, and herbals.

As used herein, the phrase “pharmaceutically acceptable carrier” refers to pharmaceutically acceptable materials, compositions or vehicles, such as a liquid or solid filler, diluents, excipients, solvents or encapsulating materials, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. Pharmaceutically acceptable carriers are, within the scope of sound medical judgment, suitable for use in contact with the tissues of human beings and animals without excessive toxicity, irritation, allergic response, or other problems or complications commensurate with a reasonable benefit/risk ratio.

The term “plurality,” as used herein, means more than one, such as at least 2, 20, 50, 100, 1000, 10000, 100000, 1000000, 10000000 or even more.

As used herein, the term “powder” refers to a composition which consists of free flowing, finely dispersed solid particles that can be dispersed easily in an inhaler, can be inhaled by a person, and can reach the lungs of the person. The powder is thus referred to as “inhalable.”

As used herein, the terms “recreational agent” or “recreational substance” or variations thereof, refer to compounds useful in providing relaxing, enjoyable, and entertaining activity for a subject.

The term “subject,” as used herein, can be a human, non-human primate, or other mammal, such as but not limited to dog, cat, horse, cow, pig, turkey, goat, fish, monkey, chicken, rat, mouse, and sheep.

As used herein, the term “sterol” includes all sterols without limitation, for example: sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, cholesterol and all natural or synthesized forms and derivatives thereof, including isomers. It is to be understood that modifications to the sterols i.e. to include side chains also falls within the purview of this invention.

The term “therapeutic agent” refers to any substance having therapeutic properties that produce a desired, usually beneficial, effect. For example, therapeutic agents may treat, ameliorate, and/or prevent disease. Such agents can be synthetic or naturally occurring, non-peptide, proteins or peptides, oligonucleotides or nucleotides, polysaccharides or sugars.

As used herein, the term “therapeutically effective amount” is an amount sufficient to contribute to the treatment, prevention, or reduction of a symptom or symptoms of a condition. A reduction of a symptom or symptoms (and grammatical equivalents of this phrase) means decreasing of the severity or frequency of the symptom(s), or elimination of the symptom(s).

“Vesicles” are defined herein as a type of a lipid-polymer composite particle in which amphipathic molecules (e.g., lipids) collectively define a volume, e.g., a substantially spherical volume. Amphipathic molecules (e.g., lipids) typically make up at least one shell of a vesicle. In this shell, the amphipathic molecules are arranged in a bilayer with hydrophilic portions of the amphipathic molecules being outwardly directed relative to the plane of the bilayer and the hydrophobic portions of the amphipathic molecules being disposed predominantly within the bilayer. The converse arrangement exists if the surrounding medium is hydrophobic.

As used herein, the term “vulvovaginal surface” herein denotes any external or internal surface of the female genitalia, including mucosal surfaces in the vaginal cavity and non-mucosal surfaces of the vulva and immediately surrounding areas of skin. In some embodiments, the composition is adapted for application to a vaginal mucosal surface, and at least a portion of the composition is bioadhesive, i.e., mucoadhesive to the vaginal mucosal surface.

### BRIEF DESCRIPTION OF THE DRAWINGS

FIG. 1 is a drawing showing the chemical structure of cannabidiol (CBD-1).

FIGS. 2A and 2B are a series of drawings showing the chemical structures of naturally occurring cannabidiol derivatives. FIG. 2A shows cannabidiolic acid (CBDA), cannabidiol monomethyl ether (CBDM), cannabidiol-C4 (CBD-C₄), and cannabidivarinic acid (CBDVA), and FIG. 2B shows cannabidivarin (CBDV), and cannabidiorcol (CBD-C₁).

FIG. 3 is a set of optical microscopic images of preliminary liposomal formulations Fa1, Fa2, and Fa3. Scale bar is 50 μm.

FIG. 4 is a set of optical microscopic images of preliminary liposomal formulations Fb1, Fb2, and Fb3 that were prepared to assess the effect of temperature on the particle formulation. Scale bar is 10 μm.

FIG. 5 is a table showing the effective diameter and polydispersity of formulations Fc1-Fc7. Formulations Fc1, Fc2, and Fc9 were loaded with 0.02% w/w CBD. Formulations Fc3-Fc8 were loaded with 0.25% w/w CBD. Mean and standard deviation values are reported.

FIG. 6 is a table showing the mean and standard deviation values of measured zeta potential of particles in formulations Fc1-Fc9.

FIG. 7 is a photographic image showing the optical transparency of formulations Fd1-Fd6. The transparency increased with increased concentration of the poloxamer PLURONIC® F127. Concentration of CBD was constant for all suspensions at 0.5% w/w while the PLURONIC® F127 concentration was 1%, 2%, 3%, 4%, 5%, and 10% for formulations Fd1, Fd2, Fd3, Fd4, Fd5, and Fd6 respectively.

FIG. 8 is a photographic image showing the effect of poloxamer concentration to the amount of observable precipitate in formulations Fd6, Fd5, Fd4, Fd2, and Fd1. Concentration of CBD was constant for all suspensions at 0.5% w/w while the PLURONIC® F127 concentration was 1%, 2%, 4%, 5%, and 10% for formulations Fd1, Fd2, Fd4, Fd5, and Fd6 respectively.

FIG. 9 is graph showing effective diameter and polydispersity of formulations Fd1-Fd6. All formulations resulted in micelles of similar size below 50 μm.

FIG. 10 is graph showing the pH of formulations Fd1-Fd6 in comparison to the pH of commercially available eye drop solutions THEALOZ® Duo and HYABAK®.

FIG. 11 is graph showing the viscosity of formulations Fd1-Fd6 in comparison the viscosity of water and two commercially available eye drop solutions THEALOZ® Duo and HYABAK®.

FIG. 12 is graph showing the size stability of formulations Fd1-Fd6. The effective diameter and polydispersity were calculated for all formulations on the day of preparation (bar on the left), and 30 days after preparation when stored in either room temperature 20° C. (bar in the middle) or at 4° C. (bar on the right).

FIG. 13 is a photographic image showing the optical transparency of formulations Fe1-Fe9. Formulations Fe1, Fe4, and Fe7 were prepared by poloxamer homogenization followed by 0.5% CBD w/w and phospholipid and cholesterol added via ethanol injection. Formulations Fe2, Fe5, and Fe8 were prepared by poloxamer homogenization with 0.5% CBD w/w followed by phospholipid and cholesterol addition via ethanol injection. Formulations Fe3, Fe6, and Fe9 were prepared by poloxamer homogenization with 0.5% CBD w/w followed by phospholipid and cholesterol addition in powder form.

FIG. 14 is graph showing the particle size, polydispersity, and span values of lipid-polymer composite particles in formulations Fe1-Fe9.

FIG. 15 is graph showing the pH of formulations Fe1-Fe9. All formulations had pH near neutral.

FIG. 16 is table showing the tonicity of formulations Fe1-Fe9 compared to the tonicity of saline, control poloxamer and CBD suspensions and three commercially available eye drop solutions HYABAK® 0.15%, THEALOZ® Duo New, and THEALOZ® Duo.

FIG. 17 is graph showing is graph showing the size stability of formulations Fe1-Fe9. The effective diameter and polydispersity were calculated for all formulations on the day of preparation (bar on the left), and 19 days after preparation when stored in either room temperature 20° C. (bar in the middle) or at 4° C. (bar on the right).

FIG. 18 is graph showing particle size, polydispersity, and span values of optimized formulations Fd5, Fe5, Fe8, and Fe2.

FIG. 19 is a graph showing particle size and polydispersity before (blue) and after (grey) filtration of the CBD formulations Fd5a, Fe5a, Fe8a, and Fe2a.

FIG. 20 is a graph showing the pH of CBD formulations Fd5a, Fe5a, Fe8a, and Fe2a before (blue) and after (orange) filtration.

FIG. 21 is a graph showing the size and polydispersity of lipid-polymer nanoparticles of formulations Fe5 and Fe8 following lipid injection at 45° C.

FIG. 22 is a graph showing the size and polydispersity of lipid-polymer nanoparticles of formulations Ff1 and Ff2.

FIG. 23 is a photographic image showing the visual appearance of formulations Fd5, Fe5, and Fe8.

FIG. 24 is a graph showing the size and polydispersity of lipid-polymer nanoparticles of formulations Fd5, Fe5 and Fe8 after 117 days stored in 3° C.

FIG. 25 is a graph showing the size and polydispersity of lipid-polymer nanoparticles of formulations Fd5, Fe5 and Fe8 after 132 days stored in 25° C. and 60% relative humidity.

FIG. 26 is a photographic image showing the visual appearance of lipid-polymer nanoparticles of formulations Fd5, Fe5 and Fe8 after 132 days stored in 25° C. and 60% relative humidity.

FIG. 27 is a graph showing the pH of lipid-polymer nanoparticles of formulations Fd5, Fe5 and Fe8 after 117 days stored in 3° C. (blue) and after 132 days stored in 25° C. and 60% relative humidity (orange).

FIG. 28 is a graph showing the size and polydispersity of lipid-polymer nanoparticles of formulations Fd5, Fe5 and Fe8 including citric acid.

FIG. 29 is a graph showing the size and polydispersity of lipid-polymer nanoparticles of formulations Fd5, Fe5 and Fe8 including sodium sulfite.

FIG. 30 is a graph showing the pH of lipid-polymer nanoparticles of formulations Fd5, Fe5 and Fe8 containing citric acid anhydrous (blue) or sodium sulfite (orange).

FIG. 31 is a graph showing the characterization of the melting point of formulations containing CBD only.

FIG. 32 is a graph showing the characterization of the melting point of formulations containing CBD and an antioxidant.

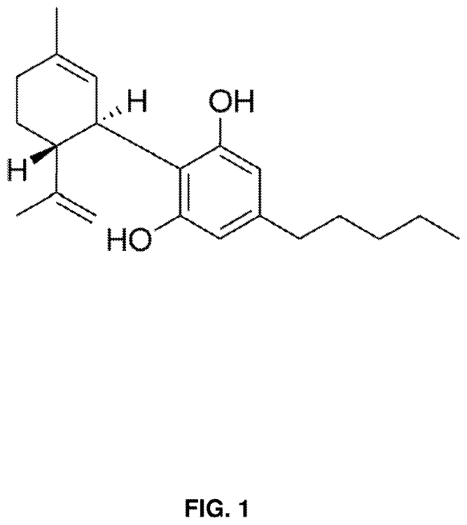

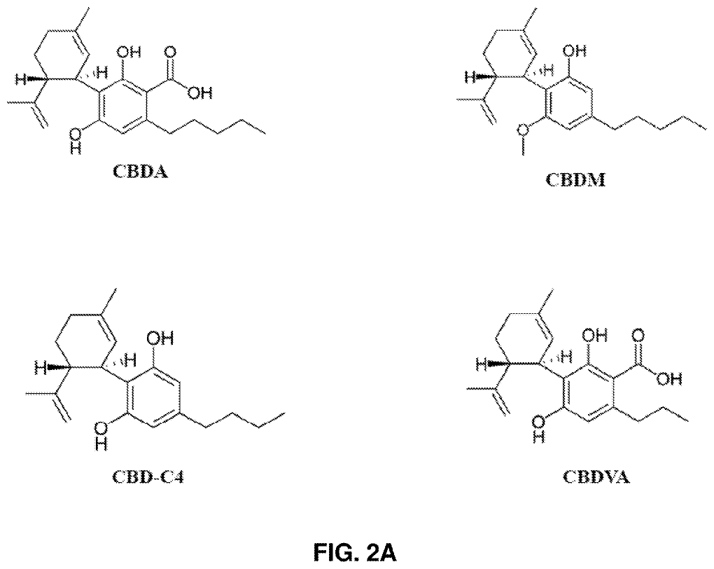

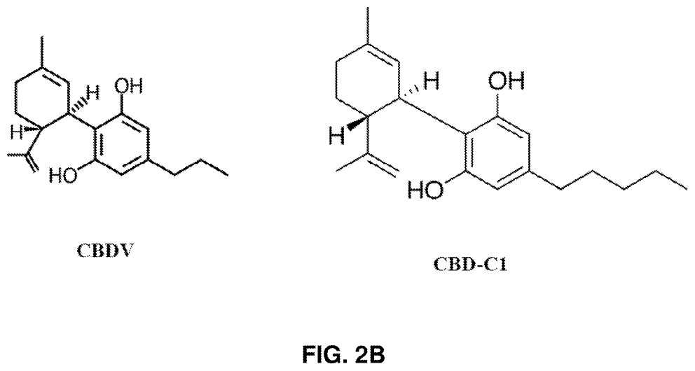

### DETAILED DESCRIPTION OF THE INVENTION

In general, the invention features cannabinoid compositions that are useful for the treatment or prevention of diseases in a subject e.g., a human subject or an animal subject. The compositions include therapeutically effective amounts of at least one cannabinoid, an analog thereof, a derivative thereof (e.g., cannabinoid, a parasympathetic agonist, and a sympathetic agonist) for the treatment or prevention of an ophthalmic condition, a dermatological condition (e.g., skin pigmentation such as hyperpigmentation (e.g., chloasma, melasma), skin lightening (e.g., cosmetic skin lightening), age spots (lentigines), freckles (ephelides), sunspots, seborrheic keratosis, acne rosacea, acne vulgaris, or dandruff (seborrheic dermatitis)), a sleep disorder (e.g., insomnia), a neurological disorder (anxiety, post-traumatic stress disorder, or a psychosomatic condition), pain (e.g., acute pain such as trauma-induced pain, dental pain, cancer-induced pain, post-surgical pain, post-herpetic neuralgia, fibromyalgia, inflammatory pain, trauma-based neuropathic pain, multiple sclerosis-induced pain, osteoarthritis, and complex regional pain syndrome (CRPS)), inflammation (e.g., autoimmune diseases, inflammation caused by bacterial, viral, or fungal infection, ankylosing spondylitis, antiphospholipid antibody syndrome, gout, inflammatory arthritis, rheumatoid arthritis, scleroderma, Sjogren's Syndrome, systemic lupus erythematosus (“SLE”), vasculitis, and asthma), diabetes (e.g., including Type I and Type II diabetes), a vision disorder (e.g., presbyopia, myopia, hypermetropia, and astigmatism), or a bacterial (e.g., bacterial vaginosis) or fungal infection (e.g., candidiasis). In some embodiments, the condition is an ophthalmic condition. In some embodiments, the condition is a dermatological condition. In some embodiments, the condition is age spots. In some embodiments, the condition is freckles. In some embodiments, the condition is sunspots. In some embodiments, the condition is seborrheic keratosis. In some embodiments, the condition is acne rosacea. In some embodiments, the condition is acne vulgaris. In some embodiments, the condition is dandruff. In some embodiments, the condition is a sleep disorder. In some embodiments, the condition is a neurological disorder. In some embodiments, the condition is pain. In some embodiments, the condition is inflammation. In some embodiments, the condition is diabetes. In some embodiments, the condition is a vision disorder. In some embodiments, the condition is a bacterial infection. In some embodiments, the condition is a fungal infection.

The present invention also features new lipid-polymer composite particles that are useful for the formulation of bioactive agents for administration to a subject, e.g., a human subject. The lipid-polymer composite particles include a plurality of (e.g., at least 2, 3, 4, 5, 6, 7, 8, 9, 10, 20, 30, 40, 50, 100, 1,000, 10,000, 100,000, 1,000,000, 10,000,000, or more) nanoparticles that encapsulate a bioactive agent. The nanoparticles include a block copolymer, a lipid, e.g., phospholipid, and a sterol. The formulations of a bioactive agent (e.g., a therapeutic agent, a nutraceutical agent, or a recreational agent) described herein provide for easier loading of lipid-polymer composite particles with higher drug loading capacity, increased stability of the formulations, and lower surface tension of water, which allows for lipid coating and entrapment. This improved process for preparing lipid-polymer composite particles, e.g., micelles, with a lipid coating allows aqueous loading of hydrophobic bioactive agents as well as controlled drug release. Furthermore, the compositions and methods described herein avoid the use of organic solvents e.g., ethanol, to solubilize hydrophobic bioactive agents. The components of the formulations are described in more detail below.

### Treatment of Diseases and Conditions Using CBD Compositions

The compositions described herein are formulated to treat an ophthalmic condition (e.g., dry eye, blepharitis, meibomianitis, keratitis, bacterial keratitis, protozoal keratitis, fungal keratitis, viral keratitis, conjunctivitis, bacterial conjunctivitis, viral conjunctivitis, episcleritis, scleritis, corneal abrasion, inflammation of the eye, injury to the eye following eye surgery (e.g., laser eye surgery), blepharoconjunctivitis, ocular rosacea, glaucoma, and contact lens intolerance, among others). In some embodiments the compositions are formulated to treat dermatological conditions (e.g., blepharitis, meibomianitis, eczema, dermatitis, atopic dermatitis, contact dermatitis, seborrheic dermatitis, perioral dermatitis, xerotic eczema, psoriasis, acne vulgaris, dryness of the skin, tinea pedis, hives, or impetigo, non-melanoma cancer, itching dermatosis, acne rosacea, aging of skin, dandruff, excessive darkening of the subject's skin, liver spot, hyperpigmentation, overexposure to ultraviolet radiation, age spot, or seborrheic keratosis). In other embodiments, the compositions are formulated to treat a vision disorder (e.g., presbyopia, hypermetropia, and astigmatism). In some embodiments, the condition is dry-eye disease. In some embodiments, the condition is blepharitis. In some embodiments, the condition is meibomianitis. In some embodiments, the condition is keratitis. In some embodiments, the condition is bacterial keratitis. In some embodiments, the condition is protozoal keratitis. In some embodiments, the condition is fungal keratitis. In some embodiments, the condition is viral keratitis. In some embodiments, the condition is conjunctivitis. In some embodiments, the condition is bacterial conjunctivitis. In some embodiments, the condition is episcleritis. In some embodiments, the condition is scleritis. In some embodiments, the condition is corneal abrasion. In some embodiments, the condition is inflammation of the eye. In some embodiments, the condition is injury to the eye following eye surgery (e.g., laser eye surgery, among others). In some embodiments, the condition is blepharoconjunctivitis. In some embodiments, the condition is ocular rosacea. In some embodiments, the condition is glaucoma. In some embodiments, the condition is eczema. In some embodiments, the condition is dermatitis. In some embodiments, the condition is atopic dermatitis. In some embodiments, the condition is contact dermatitis. In some embodiments, the condition is seborrheic dermatitis. In some embodiments, the condition is perioral dermatitis. In some embodiments, the condition is xerotic eczema. In some embodiments, the condition is psoriasis. In some embodiments, the condition is acne vulgaris. In some embodiments, the condition is dryness of the skin. In some embodiments, the condition is tinea pedis. In some embodiments, the condition is hives. In some embodiments, the condition is impetigo. In some embodiments, the condition is non-melanoma cancer. In some embodiments, the condition is itching dermatosis. In some embodiments, the condition is acne rosacea. In some embodiments, the condition is aging of the skin. In some embodiments, the condition is dandruff. In some embodiments, the condition is excessive darkening of the skin. In some embodiments, the condition is liver spot. In some embodiments, the condition is hyperpigmentation. In some embodiments, the condition is overexposure to ultraviolet radiation. In some embodiments, the condition is age spot. In some embodiments, the condition is seborrheic keratosis. In some embodiments, the condition is presbyopia. In some embodiments, the condition is hypermetropia. In some embodiments, the condition is astigmatism.

In further embodiments, the compositions are formulated to treat a vaginal infection (e.g., bacterial vaginosis or candidiasis). The pharmaceutical composition for the treatment of bacterial vaginosis or candidiasis may include therapeutically effective amounts of at least one CBD in concentrations between 0.01% to about 30% (e.g., from about 0.05% to about 9.5%, from about 0.1% to about 9%, from about 0.2% to about 8.5%, from about 0.4% to about 8%, from about 0.5% to about 7.5%, from about 1% to about 7%, from about 1.5% to about 6.5%, from about 2% to about 6%, from about 2.5% to about 5.5%, from about 3% to about 5%, from about 3.5% to about 4.5%, from about 4% to about 4.49%, e.g., about 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 22.0%, 22.2%, 22.2%, 22.3%, 22.4%, 22.5%, 22.6%, 22.7%, 22.8%, 22.9%, 22.0%, 22.2%, 22.2%, 22.3%, 22.4%, 22.5%, 22.6%, 22.7%, 22.8%, 22.9%, 23.0%, 23.2%, 23.2%, 23.3%, 23.4%, 23.5%, 23.6%, 23.7%, 23.8%, 23.9%, 24.0%, 24.2%, 24.2%, 24.3%, 24.4%, 24.5%, 24.6%, 24.7%, 24.8%, 24.9%, 25.0%, 25.2%, 25.2%, 25.3%, 25.4%, 25.5%, 25.6%, 25.7%, 25.8%, 25.9%, 26.0%, 26.2%, 26.2%, 26.3%, 26.4%, 26.5%, 26.6%, 26.7%, 26.8%, 26.9%, 27.0%, 27.2%, 27.2%, 27.3%, 27.4%, 27.5%, 27.6%, 27.7%, 27.8%, 27.9%, 28.0%, 28.2%, 28.2%, 28.3%, 28.4%, 28.5%, 28.6%, 28.7%, 28.8%, 28.9%, 29.0%, 29.2%, 29.2%, 29.3%, 29.4%, 29.5%, 29.6%, 29.7%, 29.8%, 29.9%, or 30%) by weight of the composition. The composition may be administered to the vulvovaginal surface by applying it to the surfaces in the vaginal cavity of the subject.

The compositions described herein may be administered to a subject's respiratory tract (e.g., by insufflation) and treat a sleep disorder, anxiety, post-traumatic stress disorder, a psychosomatic condition (e.g., insomnia), a painful condition, an inflammatory condition, asthma, and diabetes. In some embodiments, the condition is a sleep disorder. In some embodiments, the condition is anxiety. In some embodiments, the condition is post-traumatic stress disorder. In some embodiments, the condition is a psychosomatic condition. In some embodiments, the condition is insomnia. In some embodiments, the condition is a painful condition. In some embodiments, the condition is an inflammatory disease. In some embodiments, the condition is asthma. In some embodiments, the condition is diabetes. In some embodiments, the composition may include a powder that includes a guest particle adhered to a carrier particle. The guest particle and the carrier particle may both include at least one cannabinoid. The composition may include a sedative (e.g., an antianxiety agent). The antianxiety agent may include pentobarbital, secobarbital, diazepam, chlordiazepoxide, prazepam, clonazepam, midazolam, nitrazepam oxazepam, lorazepam, alprazolam, buspirone, flurazepam, temazepam, triazolam, chloral hydrate, zolpidem, zopiclone, eszopiclone, and diphenhydramine. The composition may further include a solubility controlling agent that includes an anionic surfactant, a cationic surfactant; a non-ionic surfactant, a zwitterionic surfactant, an amino acid, a sugar, a water-soluble polymer, a disintegrant, or a combination thereof. In some embodiments, the composition includes a carrier particle that can include lactose, D-mannitol, sorbitol, erythritol, α-trehalose dihydrate, dextrose, glucose monohydrate, maltitol, maltose, xylitol hydroxyapatite, D-raffinose anhydrous, raffinose pentahydrate, a surfactant, polyvinyl alcohol, polyvinylpyrrolidone, poly (lactic-co-glycolic acid) (PLGA)), microcrystalline cellulose (MCC), hydroxyl propyl methyl cellulose (HPMC), an amino acid, magnesium stearate or cyclodextrins. In some embodiments, the composition having two guest particles adhered to one or more carrier particle types. The compositions are administered to a subject to slowly release the pharmaceutical composition into a subject's lungs.

A subject with a disease or disorder as described herein may be administered the composition at least twice a day, at least once a day, at least once every two days, at least once every three days, or at least once a week. The pharmaceutical composition may be administered each day for at least seven days.

In some embodiments where a subject is treated for a dermatological condition, a composition (e.g., topical composition) is administered to the subject's skin by applying the composition to the outer portion of the eyelid of the subject.

### Cosmetic Treatment Using CBD Compositions

The compositions may also be formulated to provide cosmetic treatment (e.g., skin lightening, wrinkles, and fine lines). In some embodiments for cosmetic skin treatment, CBD is present in the pharmaceutical composition at a concentration from about 0.01% to about 10% (e.g., from about 0.05% to about 9.5%, from about 0.1% to about 9%, from about 0.2% to about 8.5%, from about 0.4% to about 8%, from about 0.5% to about 7.5%, from about 1% to about 7%, from about 1.5% to about 6.5%, from about 2% to about 6%, from about 2.5% to about 5.5%, from about 3% to about 5%, from about 3.5% to about 4.5%, from about 4% to about 4.49%, e.g., about 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0%) by weight of the composition. A skin lightening agent may be included in the composition and the skin lightening agent may be selected from the group consisting of hydroquinone, L-ascorbic acid, glycolic acid, lactic acid, arbutin, kojic acid, daisy flower extract, licorice extract, and placenta extract. The skin lightening agent in the composition may have a concentration between 2% and 12% (e.g., 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, or 12.0%). The composition for cosmetic treatment may also include a vitamin selected from the group consisting of vitamin A, vitamin B, vitamin C, vitamin D, and vitamin E. The composition for cosmetic treatment may also include an agent for treating acne selected from the group consisting of salicylic acid, glycolic acid, or lactic acid. The composition for cosmetic treatment may also include a retinoid to treat wrinkles and/or fine lines.

### Pharmaceutical Compositions

The compositions described herein are preferably formulated into pharmaceutical compositions for administration to human subjects in a biologically compatible form suitable for administration in vivo. The pharmaceutical compositions can be formulated with a pharmaceutically acceptable carrier or excipient. A pharmaceutically acceptable carrier or excipient refers to a carrier (e.g., carrier, media, diluent, solvent, vehicle, etc.) which does not significantly interfere with the biological activity or effectiveness of the active ingredient(s) of a pharmaceutical composition and which is not excessively toxic to the host at the concentrations at which it is used or administered. Typical pharmaceutically acceptable carriers for the compositions disclosed herein include, for example, water, mixtures of water and water-miscible solvents such as lower alkanols or aralkanols, vegetable oils, polyalkylene glycols, petroleum-based jelly, ethyl cellulose, ethyl oleate, carboxymethylcellulose, polyvinylpyrrolidone, isopropyl myristate, and other conventionally employed acceptable carriers.

In some embodiments pharmaceutically acceptable excipients or carriers include white soft paraffin, liquid paraffin, propylene carbonate, white beeswax, hard paraffin, butylhydroxytoluene (E321), all-rac-α-tocopherol, at least one of a fat, a siloxane, an emollient, an emulsifier, alcohol, polyol, polyolether, penetration enhancer, or a combination thereof any of the foregoing. In some embodiments, pharmaceutically acceptable dispersant includes lecithin and glycerin. In certain embodiments, at least one of the fats is selected from the group consisting of lard, butter, palm oil, shea butter, mango butter, kokum butter, cocoa butter, decanoic acid, undecanoic acid, erucic acid, tetradeconol, tridecanal, lauryl alcohol, beneicosane, monodecane, octadecane, eicosane, elemi resin, levulinic acid, coconut oil, dimethyl sebacate, adipic acid, polyethylene glycol, diethylene glycol, monotetradecyl ether, diethylene glycol, heptaethycine glycol monododecyl ether, palmitate esters, stearate esters, polycaprolactone-block-polytetrahydro-furan-block-poly[di(ethyleneglycol)-adipate], hydrogenated oils, squalane, petroleum, solid paraffin, carnauba wax, bees wax, lanolin, trilaurin, stearic acid, palmitic acid, capric acid, myristic acid, lauric acid, tallow, whale blubber, and combinations thereof. In certain embodiments, at least one emollient is selected from the group consisting of lanolin, mineral oil, paraffin, petrolatum, red petrolatum, white ointment, white petrolatum, yellow ointment, castor oil, cocoa butter, coconut oil, corn oil, cottonseed oil, olive oil, peanut oil, persic oil, sesame oil, cetyl esters wax, cold cream, hydrophilic ointment, rose water ointment, cetyl alcohol, glycerin, hydrophilic petrolatum, isopropyl myristate, myristyl alcohol, oleyl alcohol, shark liver oil, and combinations thereof. The pharmaceutical compositions may include alkaloids, lignans, or a combination of alkaloids and lignans. Pharmaceutical compositions described herein may also include an agent (e.g., thickening agent) selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, and combinations thereof. A solubilizing agent selected from ethanol, glycerin, propylene glycol, polyethylene glycol, copolymers of ethylene oxide and propylene glycol, and combinations thereof may also be included in the pharmaceutical composition.

In some embodiments, the pharmaceutical composition may include UV ray filters. Suitable UV ray filter for the topical composition may be butylene glycol dicaprylate or dicaprate, ethylhexyl methoxycinnamate, bis-ethylhexylloxyphenol methoxyphenyl triazine, camphor benzalkonium methosulfate, diethylhexyl butamido triazone, bisdisulizole disodium, drometrizole trisiloxane, ethylhexyl triazone, methyl anthranilate, 4-methylbenzylidene camphor, methylene bis-benzotriazolyl tetramethylbutyl phenol, octocrylene, butyl methoxydibenzoylmethane, phenylbenzimidazole sulfonic acid, polyacrylamidomethyl benzylidene camphor, or terephthalylidene dicamphor sulfonic acid. UV ray filters may be added into the topical composition in embodiments used during the day, when skin exposure to sunlight is expected.

In some embodiments, the pharmaceutical composition also contains non-toxic auxiliary substances such as emulsifying, preserving, wetting agents, bodying agents and the like, such as for example, polyethylene glycols 200, 300, 400, and 600, carbowaxes 1,000, 1,500, 4,000, 6,000, and 10,000, antibacterial components such as quaternary ammonium compounds, phenylmercuric salts known to have cold sterilizing properties and which are non-injurious in use, thimerosal, benzalkonium chloride, methyl and propyl paraben, benzododecinium bromide, benzyl alcohol, phenylethanol, buffering ingredients such as sodium chloride, sodium borate, sodium acetate, or gluconate buffers, and other conventional ingredients such as dextrose, maltodextrin, glycerol, ethanol, sorbitan monolaurate, triethanolamine, polyoxyethylene sorbitan monopalmitylate, dioctyl sodium sulfosuccinate, monothioglycerol, thiosorbitol, ethylenediamine tetra-acetic acid, and the like. Additionally, suitable vehicles are used as carrier media in some embodiments including conventional phosphate buffer vehicle systems, isotonic boric acid vehicles, isotonic sodium chloride vehicles, isotonic sodium borate vehicles and the like. Other pharmaceutically acceptable ingredients can be present in the composition as well. Suitable substances and their use for the formulation of pharmaceutically active compounds are well-known in the art (see, for example, Remington: The Science and Practice of Pharmacy. 21st Edition. Philadelphia, PA. Lippincott Williams & Wilkins, 2005, for additional discussion of pharmaceutically acceptable substances and methods of preparing pharmaceutical compositions of various types). In some embodiments, the pharmaceutical compositions may include micellar water. A therapeutically effective concentration of a cannabinoid and a bioactive agent herein described may be also included in the pharmaceutical compositions that include micellar water.

A pharmaceutical composition is typically formulated to be compatible with its intended route of administration. For oral administration, agents can be formulated by combining the bioactive agent with pharmaceutically acceptable carriers well known in the art. Such carriers enable the agents of the invention to be formulated as a powder, tablet, pill, capsule, lozenge, liquid, gel, syrup, slurry, suspension, and the like. It is recognized that some pharmaceutical compositions, if administered orally, must be protected from digestion. This is typically accomplished either by complexing the protein with a composition to render it resistant to acidic and enzymatic hydrolysis or by packaging the protein in an appropriately resistant carrier such as a liposome. Suitable excipients for oral dosage forms include, for example, fillers such as sugars, including lactose, sucrose, mannitol, or sorbitol; cellulose preparations such as, for example, starch, gelatin, gum tragacanth, methyl cellulose, hydroxypropylmethyl cellulose, sodium carboxymethylcellulose, and/or polyvinylpyrrolidone (PVP). Disintegrating agents may be added, for example, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

For administration by inhalation, pharmaceutical compositions may be formulated in the form of an aerosol spray from a pressured container or dispenser, which contains a suitable propellant, e.g., a gas such as carbon dioxide, a fluorocarbon, or a nebulizer. Liquid or dry aerosol (e.g., dry powders, large porous particles, etc.) can also be used. For topical application, a pharmaceutical composition may be formulated in a suitable ointment, lotion, gel, or cream containing the active components suspended or dissolved in one or more pharmaceutically acceptable carriers suitable for use in such compositions. Compositions formulated for ocular administration may be formulated, e.g., with hyaluronic acid.

The compositions described herein may further include one or more sterols. Sterols are lipids that are often found naturally in plants, animals, and fungi. Phytosterols refers to a class of plant sterol molecules, which are naturally occurring compounds found in plant cell membranes. Phytosterols include both plant sterols and stanols. Phytosterols may be derived from any common plant source, such as soy, wood, tall oil, vegetable oil, and the like. Phytosterols include β-sitosterol, campesterol, stigmasterol, stigmastanol, campestanol, brassicasterol, ergosterol, lupeol, cycloartenol, and the like. In some embodiments β-sitosterol is present in the pharmaceutical composition at a concentration from about 0.01% to about 10% (e.g., from about 0.05% to about 9.5%, from about 0.1% to about 9%, from about 0.2% to about 8.5%, from about 0.4% to about 8%, from about 0.5% to about 7.5%, from about 1% to about 7%, from about 1.5% to about 6.5%, from about 2% to about 6%, from about 2.5% to about 5.5%, from about 3% to about 5%, from about 3.5% to about 4.5%, from about 4% to about 4.49%, e.g., about 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0%) by weight of the composition.

The compositions described herein may be administered to a subject in a variety of forms depending on the selected route of administration, as will be understood by those skilled in the art. The compositions described herein may be administered, for example, by any route that allows the composition to reach the target cells. The composition may be administered, for example, by oral, topical, parenteral, intrathecal, intracerebroventricular, intraparenchymal, buccal, sublingual, nasal, rectal, patch, pump, transdermal, sublingual, vaginal, ocular, otic, or nasal administration and the pharmaceutical compositions formulated accordingly. The composition may be administered via inhalation or nebulization. Parenteral administration includes intravenous, intraperitoneal, subcutaneous, intramuscular, transepithelial, nasal, intrapulmonary, rectal, and topical modes of administration (e.g., via a foam, a cream, a paste, a gel, an aerosol, an ointment, a shampoo, or a lotion).

In some embodiments, the compositions described herein are formulated as part of a food or beverage conveyance, e.g., that can be administered as a food or with a meal. In some embodiments, the compositions may be formulated for edible consumption by humans or animals (e.g., meat production, fish production). In some embodiments, flavoring oils, such as apple, cherry, green tea, cinnamon, clove, black tea, plum, mango, date, watermelon, coconut, pear, jasmine, peach, fennel, fragrant melon, lychee, mint, chocolate, coffee, cream, banana, almond, grape, strawberry, blueberry, blackberry, pine, kiwi, sapote, taro, lotus, pineapple, orange, lemon, melon, peach, licorice, vanilla, rose, osmanthus, kiwi, ginseng, spearmint, citrus, cucumber, honeydew, walnut, almond, honey, or any suitable flavor, may be added into the composition. In some embodiments, the compositions are formulated as drinks (e.g., alkaline water, ozone water, tea, instant tea powder, iced tea, coffee, soft drinks, carbonated seltzers, beer, liquor, energy drinks, or juices). In some embodiments, the compositions are formulated as confectionary products (e.g., candy, gummy, jelly, cream, ice cream, whipped cream, ice, popsicle, chewing gum, syrup, chocolate-based products, hazelnut-based products, peanut based products, or corn-based products).

In some embodiments, the compositions described herein are formulated as personal care products (e.g., shampoo, soap, body wash, bubble bath, face wash, make up removal products, conditioning creams, mouthwash, toothpaste, sunscreen, lotion, perfume, cologne, deodorant, antiperspirant, shaving cream, aftershave, hair gel, hair spray, or hair mousse). In some embodiments, the compositions described herein are formulated as recreational products (e.g., tobacco products, vapes, essential oils, mists, or aromatherapy).

In general, the dosage of a pharmaceutical composition (e.g., of the bioactive agent) may be in the range of from about 1 ng to about 1 g (e.g., from about 1 ng to about 10 ng, e.g., 2 ng, 3 ng, 4 ng, 5 ng, 6 ng, 7 ng, 8 ng, 9 ng, 10 ng, e.g., 10 ng-100 ng, e.g., 20 ng, 30 ng, 40 ng, 50 ng, 60 ng, 70 ng, 80 ng, 90 ng, 100 ng, e.g., from about 100 ng to about 1 μg, e.g., 200 ng, 300 ng, 400 ng, 500 ng, 600 ng, 700 ng, 800 ng, 900 ng, 1 μg, e.g., from about 1 μg to about 10 μg, e.g., 1 μg, 2 μg, 3 μg, 4 μg, 5 μg, 6 μg, 7 μg, 8 μg, 9 μg, 10 μg, e.g., from about 10 μg to about 100 μg, e.g., 20 μg, 30 μg, 40 μg, 50 μg, 60 μg, 70 μg, 80 μg, 90 μg, 100 μg, e.g., 100 μg-1 mg, e.g., 200 μg, 300 μg, 400 μg, 500 μg, 600 μg, 700 μg, 800 μg, 900 μg, 1 mg, e.g., from about 1 mg to about 10 mg, e.g., 2 mg, 3 mg, 4 mg, 5 mg, 6 mg, 7 mg, 8 mg, 9 mg, 10 mg, e.g., from about 10 mg to about 100 mg, e.g., 20 mg, 30 mg, 40 mg, 50 mg, 60 mg, 70 mg, 80 mg, 90 mg, 100 mg, e.g., from about 100 mg to about 1 g, e.g., 200 mg, 300 mg, 400 mg, 500 mg, 600 mg, 700 mg, 800 mg, 900 mg, or 1 g).

The dosage of the pharmaceutical composition (e.g., of the bioactive agent) may be administered per kg of bodyweight of the subject. For example, the dosage may be from about 0.01 mg/kg to about 100 mg/kg, e.g., about 0.01 mg/kg to about 30 mg/kg, e.g., from about 0.01 mg/kg to about 10 mg/kg, from about 0.1 mg/kg to about 1 mg/kg, e.g., about 0.02 mg/kg, 0.03 mg/kg, 0.03 mg/g, 0.04 mg/kg, 0.05 mg/kg, 0.06 mg/kg, 0.07 mg/kg, 0.08 mg/kg, 0.08 mg/kg, 0.09 mg/kg, 0.1 mg/kg, 0.2 mg/kg, 0.3 mg/kg 0.4 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 65 mg/kg, 70 mg/kg, 75 mg/kg, 80 mg/kg, 85 mg/kg, 90 mg/kg, 95 mg/kg, or 100 mg/kg. The foregoing dosages may be administered once per day, week, month, or year.

The dosage of the compositions (e.g., a composition including a bioactive agent) described herein, can vary depending on many factors, such as the pharmacodynamic properties of the agent, the mode of administration, the age, health, and weight of the recipient, the nature and extent of the symptoms, the frequency of the treatment, and the type of concurrent treatment, if any, and the clearance rate of the composition in the animal to be treated. The compositions described herein may be administered initially in a suitable dosage that may be adjusted as required, depending on the clinical response. In some embodiments, the dosage of a composition (e.g., a composition including a bioactive agent) is a prophylactically or a therapeutically effective amount. Furthermore, it is understood that all dosages may be continuously given or divided into dosages given per a given time frame. The composition can be administered, for example, every hour, day, week, month, or year. In some embodiments, the composition may be administered continuously or systemically.

The compositions described herein may be prepared using a multi-step process. In some embodiments, the sequence of steps in the process provide optimal particle size, polydispersity, solution transparency, pH, tonicity, size distribution, stability, and loading of the bioactive agent. In some embodiments, the bioactive agent is homogenized with the polymer in a first step, e.g., at a temperature of from about 50° C. to about 70° C., e.g., about 60° C. In a second step, a solution containing a lipid and a sterol is added to the homogenized bioactive and polymer suspension using an immersed injection (e.g., ethanol injection).

### Bioactive Agents

The pharmaceutical compositions comprising CBD described herein may include one or more bioactive agents. Bioactive agents may include a therapeutic agent, a nutraceutical agent, or a recreational agent. In some embodiments the bioactive agent is one or more, but not limited to, an antibiotic, an antiseptic agent, an antifungal, an antibacterial agent, an analgesic, an antiviral agent, an antiprotozoal agent, a steroid or a non-steroidal anti-inflammatory agent. Additional exemplary bioactive agents may be a terpene, a flavonoid, an-inflammatory agent, a lipophilic drug, an anti-VEGF agent, or an anti-glaucoma agent.

In some embodiments, the terpene is myrcene, beta-caryophyllene, linalool, alpha pinene, beta-pinene, ocimene, terpinolene, ocimene, terpinolene, alpha terpineol, alpha terpinene, gamma terpinene, alpha phellandrene, cymene, camphene, delta-3-carene, fenchol, 1,8-cineole, nerolidol, borneol, eucalyptol, camphene, or limonene. In some embodiments the terpene is present in the pharmaceutical composition at a concentration from about 0.01% to about 10% (e.g., from about 0.05% to about 9.5%, from about 0.1% to about 9%, from about 0.2% to about 8.5%, from about 0.4% to about 8%, from about 0.5% to about 7.5%, from about 1% to about 7%, from about 1.5% to about 6.5%, from about 2% to about 6%, from about 2.5% to about 5.5%, from about 3% to about 5%, from about 3.5% to about 4.5%, from about 4% to about 4.49%, e.g., about 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0%) by weight of the composition.

In some embodiments, the flavonoid is selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, or isoflavonoids. In some embodiments, the bioactive compound is nicotine, a nicotine analogue or a nicotine derivative. In other embodiments, the bioactive agent is cyclosporin A, tacrolimus, isotretinoin, propofol, griseofulvin, azithromycin, or a non-steroidal anti-inflammatory drug (NSAID).

In some embodiments, the bioactive agent is a cannabinoid or a cannabinoid derivative. In more preferred embodiments, the cannabinoid or derivative thereof is one or more of cannabigerolic acid (CBGA), cannabigerolic acid monomethyl ether (CBGAM), cannabigerol (CBG), cannabigerol monomethyl ether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), cannabidiorcol (CBD-C1), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C4 (THCA-C4), delta-9-tetrahydrocannabinol-C4 (THC-C4), delta-9-tetrahydrocannabivarinic acid (THCVA), delta-9-tetrahydrocannabivarin (THCV), delta-9-tetrahydrocannabiorcolic acid (THCA-C1), delta-9-tetrahydrocannabiorcol (THC-C1), delta-7-cis-iso-tetrahydrocannabivarin, delta-8-tetrahydrocannabinolic acid (Δ⁸-THCA), delta-8-tetrahydrocannabinol (Δ⁸-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methyl ether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-metha-no-2H-1-benzoxocin-5-methanol (OH-iso-HHCV), cannabiripsol (CBR) and trihydroxy-delta-9-tetrahydrocannabinol (triOH-THC). In some embodiments, the cannabinoid derivatives are naturally occurring (e.g., phytocannabinoids such as cannabidiol). In some embodiments, the cannabinoid derivatives are non-naturally occurring (e.g., synthetic cannabinoids), including those that are chemically or enzymatically synthesized. The cannabinoid of the composition includes water soluble cannabinoids and an aqueous solution including at least one water soluble cannabinoid and saline.

The bioactive agent of the compositions described herein may be encapsulated by a plurality of lipid-polymer composite particles described herein. In some embodiments the bioactive agent (e.g., cannabinoid or a derivative thereof) is present in the pharmaceutical composition at a concentration from about 0.01% to about 10% (e.g., from about 0.05% to about 9.5%, from about 0.1% to about 9%, from about 0.2% to about 8.5%, from about 0.4% to about 8%, from about 0.5% to about 7.5%, from about 1% to about 7%, from about 1.5% to about 6.5%, from about 2% to about 6%, from about 2.5% to about 5.5%, from about 3% to about 5%, from about 3.5% to about 4.5%, from about 4% to about 4.49%, e.g., about 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0%) by weight of the composition. In some embodiments, the weight ratio between the poloxamer of a lipid-polymer particle and the bioactive agent is from about 4 to about 8 (e.g., from about 4.1 to about 7.9, or from about 4.2 to about 7.8, from about 4.3 to about 7.7, from about 4.4 to about 7.6, from about 4.5 to about 7.5, from about 4.6 to about 7.4, from about 4.7 to about 7.3, from about 4.8 to about 7.2, from about 4.9 to about 7.1, from about 5.0 to about 7.0, from about 5.1 to about 6.9, from about 5.2 to about 6.8, from about 5.3 to about 6.7, from about 5.4 to about 6.6, from about 5.5 to about 6.5, from about 5.6 to about 6.4, from about 5.7 to about 6.3, from about 5.8 to about 6.2, or from about 5.9 to about 6.1).

The bioactive agent of the compositions described herein may be one or more of the following analgesic agents, but not limited to, methyl salicylate, codeine, morphine, methadone, pethidine, buprenorphine, hydromorphine, levorphanol, oxycodone, fentanyl, tramadol, and a non-steroidal anti-inflammatory drug. In some embodiments methyl salicylate is present in the pharmaceutical composition at a concentration from about 0.01% to about 10% (e.g., from about 0.05% to about 9.5%, from about 0.1% to about 9%, from about 0.2% to about 8.5%, from about 0.4% to about 8%, from about 0.5% to about 7.5%, from about 1% to about 7%, from about 1.5% to about 6.5%, from about 2% to about 6%, from about 2.5% to about 5.5%, from about 3% to about 5%, from about 3.5% to about 4.5%, from about 4% to about 4.49%, e.g., about 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0%) by weight of the composition.

Exemplary antibiotics that may be used as a bioactive agent in the compositions described herein to treat or prevent an ophthalmic condition include, but are not limited to, ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, ticarcillin clavulanic acid, nafcillin, procaine penicillin, cefadroxil, cefazolin, cephalexin, cephalothin, cephapirin, cephradine, cefaclor, cefamandole, cefonicid, cefotetan, cefoxitin, cefprozil, cefmetazole, cefuroxime, loracarbef cefdinir, ceftibuten, cefoperazone, cefixime, cefotaxime, cefpodoxime proxetil, ceftazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, pefloxacin, imipenem-cilastatin, meropenem, aztreonam, macrolides, and cyclosporin.

Exemplary antiseptic compounds include, but are not limited to, iodine, manuka honey, octenidine dihydrochloride, phenol, polyhexanide, sodium chloride, sodium hypochlorite, calcium hypochlorite, sodium bicarbonate, methyl paraben, and sodium dehydroacetate.

Exemplary antifungal agents include, but are not limited to, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, tolnaftate, undecylenic acid, crystal violet, and balsam of Peru.

Exemplary antiviral agents include, but are not limited to, acyclovir, famciclovir, penciclovir, valacyclovir, trifluridine, docosanol, amantadine, remdesivir, rimantadine, oseltamivir, and zanamivir.

Additional exemplary bioactive agents include but are not limited to, cyclodextrin, 4-Terpineol, crotamiton or permethrin, metronidazole, ivermectin, doramectin and milbemycin, rotenone ((2R,6aS,12aS)-1,2,6,6a,12,12a-hexahydro-2-isopropenyl-8,9-dimethoxychromeno[3,4-b]furo(2,3-h)chromen-6-one), amitraz (N,N′-[(Methylimino)dimethylidyne]di-2,4-xylidine), afoxolaner, BROLENE®, chlorhexidine, polyhexamethylene biguanide (PHMB), fluralaner, tacrolimus, cyclosporine, sirolimus, derivatives of Melaleuca alternifolia (Tea tree Oil), derivatives of aloe vera, methylglyoxal.

Exemplary steroids include hydrocortisone, cortisone acetate, prednisone, prednisolone, methylprednisolone, dexamethasone, betamethasone, triamcinolone, and beclomethasone, fluorometholone. Other glucocorticoids useful in the method for treating blepharitis include, for example, 21-acetoxypregnenolone, alelometasone, algestone, amcinonide, budesonide, chloroprednisone, clobetasol, clobetasone, clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, rimexolone, tixocortol, triamcinolone acetonide, triamcinolone benetonide, triamcinolone hexacetonide, their ophthalmically acceptable salts, combinations thereof, and mixtures thereof. In one embodiment, the glucocorticoid includes dexamethasone, prednisone, prednisolone, methylprednisolone, medrysone, triamcinolone, loteprednol etabonate, ophthalmically acceptable salts thereof, combinations thereof, and mixtures thereof. Exemplary steroids also include androgens, such as testosterone and androstenedione, which have been known to effect dry eye.

Exemplary non-steroidal anti-inflammatory agents include celecoxib, rofecoxib, lumiracoxib, valdecoxib, parecoxib, etoricoxib, CS-502, JTE-522, L-745,337, NS398, aspirin, acetaminophen (considered to be an NSAID for the purposes of the present disclosure), ibuprofen, flurbiprofen, ketoprofen, naproxen, oxaprozin, etodolac, indomethacin, ketorolac, lornoxicam, meloxicam, piroxicam, droxicam, tenoxicam, nabumetone, diclofenac, meclofenamate, mefenamic acid, diflunisal, sulindac, tolmetin, fenoprofen, suprofen, benoxaprofen, aceclofenac, tolfenamic acid, oxyphenbutazone, azapropazone, phenylbutazone, or combinations thereof.

The compositions described herein may also include a parasympathetic agonist. Parasympathetic agonists generally refer to cholinergic agonists that have affinity and efficacy at the cholinergic receptors of the parasympathetic neurons and tend to increase activity in those neurons. In some embodiments, useful parasympathetic agonists include muscarinic agonists. Parasympathetic agonists may be direct or indirect agonists. Pilocarpine has been used as an isolated medication for the treatment of presbyopia and mild hyperopia but has not been very effective because topical concentrations below 0.5% produce minimal effect in the accommodation of the eye and concentrations above 0.5% are not tolerated due to side effects such as red eyes, ocular pain, brow ache, and headache. In addition, at concentrations of pilocarpine effective enough to improve the reading ability of the presbyopic patient, the eye is rendered so myopic that there is a significant decrease in the eye's distance vision (Gilmartin et al., 1995, Ophthalmic and Physiological Optics, Pergamon Press, Oxford, GB, 15(5):475-479). The present invention utilizes the therapeutic activity of pilocarpine and/or other parasympathetic agonists while simultaneously mitigating the deleterious effects associated with these compounds in order to improve therapeutic efficacy, patient comfort, and compliance. Parasympathetic agonists include, but are not limited to, bethanechol, carbamylcholine, cevimeline, carbachol and pilocarpine. Parasympathetic agonists may also include indirect agonists such as cholinesterase inhibitors. Examples of cholinesterase inhibitors include, but are not limited to, delta-9-tetrahydrocannabinol, carbamates, physostigmine, neostigmine, pyridostigmine, ambenonium, demecarium, rivastigmine, phenanthrene derivatives, galantamine, caffeine—noncompetitive, piperidines, donepezil, tacrine, edrophonium, huperzine, ladostigil, ungeremine and lactucopicrin.

The compositions described herein may also include a sympathetic agonist or antagonist. Sympathetic agonists generally refer to adrenergic agonists that have affinity and efficacy at the adrenergic receptors of the sympathetic neurons and tend to increase activity in those neurons. In some embodiments, useful sympathetic agonists include α-receptor agonists (e.g., a2-receptor agonists). Sympathetic agonists include, but are not limited to, brimonidine, clonidine, guanfacine, guanabenz, guanoxabenz, guanethidine, iopidine, tizanidine, and xylazine. Sympathetic antagonists generally refer to adrenergic antagonists that inhibit the function of adrenergic receptors of the sympathetic neurons. The antagonists tend to reduce or block the signals of the receptor agonists. In some embodiments, useful sympathetic antagonists include α-receptor blocking agents (e.g., alpha blockers). Sympathetic antagonists include, but are not limited to, dapiprazole, thymoxamine, clonidine, prazosin, propranolol, guanfacine, methyldopa, guanabenz; doxazosin, prazosin, terazosin, silodosin, alfuzosin, tamsulosin, dutasertide/tamsulosin, guanadrel, mecemylamine, and guanethidine. In some embodiments the composition includes CBD-1 as the cannabinoid, pilocarpine as the parasympathetic agonist and brimonidine as the sympathetic agonist.

The compositions described herein may also include an antioxidant selected from the group consisting of, but not limited to, citric acid, sodium sulfite, tocopherol, carnosine, N-acetylcarnosine, pyruvate, resveratrol, astaxanthin, glutathione, cysteine, cysteine ascorbate, and combinations thereof. In some embodiments, the tocopherol is selected from the group consisting of alpha tocopherol, gamma tocopherol, and a combination of alpha and gamma tocopherol. In some embodiments the tocopherol is present in the pharmaceutical composition at a concentration from about 0.01% to about 20% (e.g., from about 0.05% to about 9.5%, from about 0.1% to about 9%, from about 0.2% to about 8.5%, from about 0.4% to about 8%, from about 0.5% to about 7.5%, from about 1% to about 7%, from about 1.5% to about 6.5%, from about 2% to about 6%, from about 2.5% to about 5.5%, from about 3% to about 5%, from about 3.5% to about 4.5%, from about 4% to about 4.49%, e.g., about 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, or 20%) by weight of the composition.

Compositions described herein may further include substances and/or additives that raise the therapeutic concentration and improve bioavailability of the CBD and/or other bioactive agents. Emulsifying and suspending agents are added to an ophthalmic emulsion such as DUREZOL® (difluprednate) to enhance dispersion of the hydrophobic active ingredient from the oil phase into the aqueous mixture, creating a uniform product when shaken. Additives that improve solubility include certain surfactants, caffeine, nicotinamide derivatives and cyclodextrins. Micro-emulsions improve drug permeation across the cornea and provide extended drug release that reduces the frequency of administration. These formulations are dispersions of oil and water that require surfactants and co-surfactants to enhance stability and penetration into deeper layers of ocular structures. Additionally, micro-emulsions possess low surface tension that aids in corneal spreading and mixing with the pre-corneal tear film. Despite the advantage of extended release and residence time, potential toxicity associated with high concentrations of surfactants may restrict their use.

### Ophthalmic Formulations

Ophthalmic formulations may be prepared according to known principles and including established and pharmaceutically acceptable ingredients and may include solutions, suspensions and emulsions. Formulations may also include a thin film, an ointment, a non-aqueous solution, a solid form, a paste, a polymer, an emulsion, or an injectable formulation. Liquid ophthalmic formulations also include in-situ gel forming systems that are liquids when stored but form a gel upon application to the eye. The ophthalmic liquid formulations may be administered as eye drops or a spray (aerosol, non-aerosol, or mist). The liquid ophthalmic formulation may generally be in the range of pH 3-8, alternatively pH 4-7, alternatively pH 4-6. The ophthalmic formulation can include a buffer (e.g., borates, borate-polyol complexes, succinate, phosphate buffering agents, citrate buffering agents, acetate buffering agents, carbonate buffering agents, organic buffering agents, amino acid buffering agents, or combinations thereof). The buffer should have the capacity to be in the range of pH 3-8. Examples of buffers include acetates such as sodium acetate; phosphates such as sodium dihydrogen phosphate, disodium hydrogen phosphate, potassium dihydrogen phosphate and dipotassium hydrogen phosphate; ε-aminocaproic acid; amino acid salts such as sodium glutamate; and boric acid and a salt thereof. The buffer is generally contained in a proportion of 0.01-2.0 w/v %, preferably 0.05-0.5 w/v % relative to the entire liquid ophthalmic formulation. Borates include boric acid, salts of boric acid, other pharmaceutically acceptable borates, and combinations thereof. In some cases, borates include boric acid, sodium borate, potassium borate, calcium borate, magnesium borate, manganese borate, and other such borate salts. Polyols include any compound having at least one hydroxyl group on each of two adjacent carbon atoms that are not in trans configuration relative to each other. In some embodiments, the polyols are linear or cyclic, substituted or unsubstituted, or mixtures thereof, so long as the resultant complex is water soluble and pharmaceutically acceptable. In some instances, examples of polyols include sugars, sugar alcohols, sugar acids and uronic acids. In some cases, polyols include, but are not limited to mannitol, glycerin, xylitol and sorbitol. Phosphate buffering agents include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; ammonium phosphates such as diammonium hydrogen phosphate and ammonium dihydrogen phosphate; or a combination thereof. In some instances, the phosphate buffering agent is an anhydride. In some instances, the phosphate buffering agent is a hydrate. Citrate buffering agents may include citric acid and sodium citrate. Acetate buffering agents include acetic acid, potassium acetate, and sodium acetate. Carbonate buffering agents may include sodium bicarbonate and sodium carbonate. Organic buffering agents may include Good's Buffer, such as for example 2-(N-morpholino)ethanesulfonic acid (MES), N-(2-Acetamido)iminodiacetic acid, N-(Carbamoylmethyl)iminodiacetic acid (ADA), piperazine-N,N′-bis(2-ethanesulfonic acid (PIPES), N-(2-acetamido)-2-aminoethanesulfonic acid (ACES), β-Hydroxy-4-morpholinepropanesulfonic acid, 3-Morpholino-2-hydroxypropanesulfonic acid (MOPSO), cholamine chloride, 3-(N-morpholino)propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), piperazine-1,4,-bis (2-hydroxypropanesulphonic acid) (POPSO), 4-(2-hydroxyethyl)piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; and diethanolamine (DEA). Amino acid buffering agents may include taurine, aspartic acid and its salts (e.g., potassium salts, etc.), E-aminocaproic acid, and the like. In some embodiments, the ophthalmic formulation may include a cyclodextrin containing 6, 7, or 8 glucopyranose units, referred to as α-cyclodextrin, β-cyclodextrin, or γ-cyclodextrin respectively. Cyclodextrins have a hydrophilic exterior, which enhances water-soluble, and a hydrophobic interior which forms a cavity. In an aqueous environment, hydrophobic portions of other molecules often enter the hydrophobic cavity of cyclodextrin to form inclusion compounds. Additionally, cyclodextrins are also capable of other types of nonbonding interactions with molecules that are not inside the hydrophobic cavity. Cyclodextrins have three free hydroxyl groups for each glucopyranose unit, or 18 hydroxyl groups on α-cyclodextrin, 21 hydroxyl groups on β-cyclodextrin, and 24 hydroxyl groups on γ-cyclodextrin. In some embodiments, one or more of these hydroxyl groups are reacted with any of a number of reagents to form a large variety of cyclodextrin derivatives, including hydroxypropyl ethers, sulfonates, and sulfoalkyl ethers. The hydrophobic core of the cyclodextrins may be particularly useful for solubilizing CBDs in an aqueous solution suitable for eye drops, eye rinses, eye washes, and the like. Stabilizers also may be incorporated and include, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. In further embodiments, the chosen stabilizer changes the hydrophobicity of the formulation, improves the mixing of various components in the formulation, controls the moisture level in the formula, or controls the mobility of the phase. Surfactants also may be incorporated and include, for example, polyoxyethylene fatty acid glycerides and vegetable oils, e.g., polyoxyethylene (60) hydrogenated castor oil; and polyoxyethylene alkyl ethers and alkylphenyl ethers, e.g., octoxynol-10 and octoxynol-40, phospholipids, cholesterol, and cholesterol fatty acid esters and derivatives thereof; nonionic surfactants including, for example, polyoxyethylene fatty alcohol esters, sorbitan fatty acid esters (Spans), polyoxyethylene sorbitan fatty acid esters (e.g., polyoxyethylene (20) sorbitan monooleate (TWEEN® 80), polyoxyethylene (20) sorbitan monostearate (TWEEN® 60), polyoxyethylene (20) sorbitan monolaurate (TWEEN® 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., CREMOPHOR® RH40, CREMPHOR® A25, CREMPHOR® A20, CREMPHOR® EL) and other Cremphors, sulfosuccinates, alkyl sulphates (SLS); PEG glyceryl fatty acid esters such as PEG-8 glyceryl caprylate/caprate (LABRASOL®), PEG-4 glyceryl caprylate/caprate (LABRAFAC® Hydro WL 1219), PEG-32 glyceryl laurate (GELUCIRE® 444/14), PEG-6 glyceryl mono oleate (LABRAFIL® M 1944 CS), PEG-6 glyceryl linoleate (LABRAFIL® M 2125 CS); propylene glycol mono- and di-fatty acid esters, such as propylene glycol laurate, propylene glycol caprylate/caprate; BRIJ® 700, ascorbyl-6-palmitate, stearylamine, sodium lauryl sulfate, polyoxethylene glycerol triiricinoleate, and any combinations or mixtures thereof; anionic surfactants including, for example, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and cationic surfactants including, for example, cetyltrimethylammonium bromide, and lauryl dimethyl benzyl-ammonium chloride. The ophthalmic formulation may also include a tonicity agent, a viscosifier, a bioadhesive agent, a solubilizer, and/or a permeation enhancer. Tonicity agents may also be incorporated and include, for example, phosphate-buffered saline (PBS), Alsever's solution, Tris-buffered saline (TBS), water, balanced salt solutions (BSS), sodium chloride, potassium chloride, calcium chloride, magnesium chloride, mannitol, sorbitol, dextrose, glycerin, propylene glycol, ethanol, trehalose, and combinations thereof, may be added to the formulation to approximate physiological tonicity. Such an amount of tonicity agent will vary, depending on the particular agent to be added. In general, however, the formulations will have a tonicity agent in an amount sufficient to cause the final formulation to have an ophthalmically acceptable osmolality (generally about 150-450 mOsm). Solubilizers may be added to the solution to increase the solubility of a hydrophobic drug, such as cannabinoid and derivatives thereof. Viscosity enhancers (viscosifiers) may be included to increase the contact time with the eye surface. Co-solvents and viscosifiers may be included to improve the characteristics of the formulation. Examples include non-ionic water-soluble polymers or other compounds that can lubricate, wet, or otherwise aid in the natural tear build-up. The compounds may enhance the viscosity of the formulation and include monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P. Other examples of viscosity building agents include, but are not limited to, polysaccharides, vinyl polymers, and acrylic acid polymers. Examples of polysaccharides include hyaluronic acid and its salts, chondroitin sulfate and its salts, dextrans, and various polymers of the cellulose family. Wetting agents, such as Manuka honey, may also be included.

Ophthalmic suspensions are “dispersions of finely divided, relatively insoluble drug substances in an aqueous vehicle containing suitable suspending and dispersing agents.” Chapter 86, “Ophthalmic Preparations,” in Remington's Pharmaceutical Sciences, 8th edition, A. R. Gennaro, ed., p. 1585. The cannabinoid (and derivatives thereof) in the ophthalmic suspension is absorbed from solution and the solution concentration is replenished from retained particles in the suspension. In some embodiments, the ophthalmic suspension includes a cannabinoid, such as CBD, or derivatives thereof, water, a buffer, and a preservative. The suspension may also include a drug carrier, a tonicity agent, salts, a viscosifier (viscosity building agent), a bioadhesive agent, a suspending agent, a permeation enhancer, a preservative, an antioxidant, a chelating agent, an absorption promoter, and/or a co-solvent. The ophthalmic suspension may generally be in the range of pH 3-8, alternatively pH 4-7, alternatively pH 4-6. The average size of a suspended CBD particle may be 0.01-75 μm, alternatively 0.1-50 μm, alternatively 0.1-20 μm.

In some embodiments, the formulation may be an ophthalmic gel. The ophthalmic gel may be an in situ-gel forming system. In-situ gel forming systems are typically aqueous solutions that contain one or more polymers. The systems are a low-viscosity liquid in the container but form gels when it comes into contact with the eye. Depending on the type of polymer in the gel, the transition from liquid to gel can be triggered by a change in in temperature, pH, ionic strength, or presence of tear protein. The gelling polymer may also be a natural polysaccharide with thixotropic behavior and thermoreversible polymers that utilize one or more combinations of mechanisms such as thermal gelation, corneal nucoadhesion, lysosomal interaction, and ionic gelation. The gelling polymer may be a sulphated polysaccharide or one of its derivatives, chitosan, linezolid, carbomer, and xanthan gum. Thermoreversible polymers include but are not limited to gelatin, poly(vinyl chloride), poly(acrylonitrile), polystyrene (atactic), poly(vinyl alcohol), agarose, carrageenans, benzohydroxamic acid, cellulose derivatives, poloxamer and polysaccharides. The ocular formulation may be administered as a drop that forms a gel upon contact with the eye. The ocular formulation may include the same ingredients as the liquid formulations and also include a gelling polymer. In-situ gel formulations may contain a cannabinoid, such as CBD, or derivatives thereof, a surfactant a tonicity agent, a buffer, a preservative, a co-solvent, and/or viscosity-enhancing agents. Ophthalmic gel formulations may include a hydrophobic bioactive ingredient such as cannabinoid, such as CBD, or derivatives thereof, and may contain a viscosity enhancer such that the viscosity of the gel formulation is sufficient to retain the particles of cannabinoid, such as CBD, or derivatives thereof in the gel formulation vehicle and the particles do not settle over time. The ophthalmic gel formulation may have a viscosity in the range of about 300 cP to about 1500 cP (e.g., 350 cP, 400 cP, 450 cP, 500 cP, 550 cP, 600 cP, 650 cP, 700 cP, 750 cP, 800 cP, 850 cP, 900 cP, 950 cP, 1000 cP, 1050 cP, 1100 cP, 1150 cP, 1200 cP, 1250 cP, 1300 cP, 1350 cP, 1400 cP, 1450 cP, or 1500 cP) outside the eye. The viscosity of the gel formulation may or may not change upon contact with the eye. Gel formulations may contain a surfactant, a tonicity agent, a buffer, a preservative, a co-solvent, and/or viscosity-enhancing agents. The formulation may also contain a cation or an anion.

Formulations may also include a drug carrier, for example, an aqueous carrier that could provide short-term relief of dry-eye and other conditions. The formulations could include a phospholipid carrier or an artificial tears carrier. Artificial tears carriers may include one or more phospholipids or other compounds that lubricate the eye. Formulations may include an antioxidant such as citric acid, sodium sulfite, ascorbic acid, sodium ascorbate, tocopherol, sodium thiosulfate and sodium hydrogen sulfite. Formulations may include a chelating agent such as sodium edetate (disodium ethylenediamine tetraacetate) and sodium citrate. Formulations may include a pH adjusting agent include hydrochloric acid, citric acid, phosphoric acid, acetic acid, tartaric acid, sodium hydroxide, potassium hydroxide, sodium carbonate and sodium hydrogencarbonate. Formulations may also include a preservative such as quaternary ammonium salts such as benzalkonium chloride, benzethonium chloride and the like; cationic compounds such as chlorhexidine gluconate and the like; p-hydroxybenzoates such as methyl p-hydroxybenzoate, propyl p-hydroxybenzoate and the like; alcohol compounds such as chlorobutanol, benzyl alcohol and the like; sodium dehydroacetate; thimerosal; sorbic acid; and the like. Formulations may also include a penetration enhancer that makes the cell membranes less rigid, and therefore, more amenable to allowing the drug to enter the cell. Penetration enhancers include, but are not limited to, benzalkonium chloride and EDTA. Penetration enhancers may also be saccharide surfactants, such as dodecylmaltoside (“DDM”), and monoacyl phosphoglycerides, such as lysophosphatidylcholine. Penetration enhancers may be present in an amount ranging from about 0.001 wt. % to about 3 wt. % (e.g., about 0.001 wt. % to about 0.01 wt. %, e.g., 0.002 wt. %, 0.003 wt. %, 0.004 wt. %, 0.005 wt. %, 0.006 wt. %, 0.007 wt. % 0.008 wt. %, 0.009 wt. %, or 0.01 wt. %, e.g., about 0.01 wt. % to about 0.1 wt. %, e.g., 0.02 wt. %, 0.03 wt. %, 0.04 wt. %, 0.05 wt. %, 0.06 wt. %, 0.07 wt. %, 0.08 wt. %, 0.09 wt. %, or 0.1 wt. %, e.g., about 0.1 wt. % to about 1.0 wt. %, e.g., 0.2 wt. %, 0.3 wt. %, 0.4 wt. %, 0.5 wt. %, 0.6 wt. %, 0.7 wt. %, 0.8 wt. %, 0.9 wt. %, or 1.0 wt. %, e.g., about 1.0 wt. % to about 3.0 wt. %, e.g., 1.5 wt. %, 2.0 wt. %, 2.5 wt. %, or 3.0 wt. %).

Surfactants also may be incorporated and include, for example, polyoxyethylene fatty acid glycerides and vegetable oils, e.g., polyoxyethylene (60) hydrogenated castor oil; and polyoxyethylene alkyl ethers and alkylphenyl ethers, e.g., octoxynol-10 and octoxynol-40, phospholipids, cholesterol, and cholesterol fatty acid esters and derivatives thereof; nonionic surfactants including, for example, polyoxyethylene fatty alcohol esters, sorbitan fatty acid esters (Spans), polyoxyethylene sorbitan fatty acid esters (e.g., polyoxyethylene (20) sorbitan monooleate (TWEEN® 80), polyoxyethylene (20) sorbitan monostearate (TWEEN® 60), polyoxyethylene (20) sorbitan monolaurate (TWEEN® 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., CREMOPHOR® RH40, CREMPHOR® A25, CREMPHOR® A20, CREMPHOR® EL) and other Cremphors, sulfosuccinates, alkyl sulphates (SLS); PEG glyceryl fatty acid esters such as PEG-8 glyceryl caprylate/caprate (LABRASOL®), PEG-4 glyceryl caprylate/caprate (LABRAFAC® Hydro WL 1219), PEG-32 glyceryl laurate (GELUCIRE® 444/14), PEG-6 glyceryl mono oleate (LABRAFIL® M 1944 CS), PEG-6 glyceryl linoleate (LABRAFIL® M 2125 CS); propylene glycol mono- and di-fatty acid esters, such as propylene glycol laurate, propylene glycol caprylate/caprate; BRIJ 700, ascorbyl-6-palmitate, stearylamine, sodium lauryl sulfate, polyoxethylene glycerol triiricinoleate, and any combinations or mixtures thereof; anionic surfactants including, for example, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and cationic surfactants including, for example, cetyltrimethylammonium bromide, and lauryl dimethyl benzyl-ammonium chloride. Other surfactants include tyloxapol, PLURONIC™ F-68 (BASF, Ludwighsafen, Germany), and the poloxamer surfactants. Surfactants may be ionic or non-ionic. In one embodiment, the surfactant is non-ionic, e.g., polysorbate 80, to reduce irritation.

Oils may be incorporated in the ophthalmic formulation. For example, the formulation may include a medium chain triglyceride (MCT) oil (a triglyceride oil in which the carbohydrate chain has 8-12 carbons), a vegetable oil, a mineral oil, or mixtures thereof. MCT oil is available commercially, e.g., TCR (trade name of Société Industrielle des Oléagineux, France for a mixture of triglycerides wherein about 95% of the fatty acid chains have 8 or 10 carbons) and MIGLYOL® 812 (trade name of Dynamit Nobel, Sweden for a mixed triester of glycerine and of caprylic and capric acids). Examples of vegetable oils include soybean oil, cotton seed oil, olive oil, sesame oil and castor oil. Other higher fatty acid glycerides such as peanut oil may be used. The mineral oils may be natural hydrocarbons or their synthetic analogs. Oily fatty acids, such as oleic acid and linoleic acid, fatty alcohols, such as oleyl alcohol, and fatty esters, such as sorbitan monooleate and sucrose mono-di- or tri-palmitate, can also be used as the oil component.

### Ointments and Emulsions

In some embodiments, the formulation may be an ophthalmic ointment. Ophthalmic ointments remain popular and are a frequently prescribed dosage form. Ointments are most appropriate for use prior to sleep (e.g., bedtime instillation) because of the resulting interference with vision. Ointments offer the advantage of longer contact time and greater total drug bioavailability. Ointments may include a cannabinoid, such as CBD, or derivatives thereof and an ointment base (e.g., wax/petrolatum/oil crosslinked polymer). Ointments may optionally include a drug carrier, a surfactant, a penetration enhancer, a stabilizer, a viscosifier, and/or a preservative. Ointments may be a clear hydrophobic ointment, preferably with no water present. In some embodiments, the amount of water is a maximum of about 10 wt %, alternatively about 5 wt %, alternatively about 3 wt %, alternatively about 2 wt %, alternatively about 1 wt %, alternatively between about 0.5 wt % to about 10 wt %, alternatively between about 1 wt % to about 10 wt %, alternatively between about 1 wt % to about 5 wt %, alternatively between about 1 wt % to about 3 wt %. Ointments may also be an oil-in-water emulsion (i.e., a cream). In some embodiments, the amount of water is a maximum of about 10 wt %, alternatively about 5 wt %, alternatively about 3 wt %, alternatively about 2 wt %, alternatively about 1 wt %, alternatively between about 0.5 wt % to about 10 wt %, alternatively between about 1 wt % to about 10 wt %, alternatively between about 1 wt % to about 5 wt %, alternatively between about 1 wt % to about 3 wt %. In some embodiments, the cannabinoid is present in the composition at a concentration of about 1% (w/w) to about 10% (w/w) (e.g., 2% (w/w), 3% (w/w), 4% (w/w), 5% (w/w), 6% (w/w), 7% (w/w), 8% (w/w), 9% (w/w), or 10% (w/w)) and suspended in a non-allergic pharmaceutically acceptable carrier comprising white petrolatum at a concentration of about 10% (w/w) to about 80% (w/w) (e.g., 20% (w/w), 30% (w/w), 40% (w/w), 50% (w/w), 60% (w/w), 70% (w/w), or 80% (w/w)) mineral oil at a concentration of about 10% (w/w) to about 50% (w/w) (e.g., about 20% (w/w), 30% (w/w), 40% (w/w), or 50% (w/w)) and lanolin at a concentration of about 1% (w/w) to about 5% (w/w) (e.g., 2% (w/w), 3% (w/w), 4% (w/w), or 5% (w/w)).

An alternative to ointments is the ophthalmic suspension, such as those used in LOTEMAX® (loteprednol etabonate 0.5%) or AZOPT® (brinzolamide 1%). These include solid preparations that, when reconstituted, result in a suspension. The insoluble drug is made in a micronized form and is dispersed in a suitable vehicle that contains excipients such as suspending agents, buffers and preservatives to improve solubility and prevent irritation of the cornea. Generally, the ointments and emulsions may be prepared according to known principles and including established and pharmaceutically acceptable ingredients such as those described above.

The formulations may be in the form of an ophthalmic emulsion. Ophthalmic emulsions are a fine dispersion of minute droplets of one liquid in another in which it is not soluble or miscible. An emulsion may be a dispersion of oil in water and can be classified as either a macroemulsion or microemulsion. A macroemulsion is generally thermodynamically unstable and has a cloudy, turbid composition with an oil-droplet size of 0.5 to 100 μm. A microemulsion is thermodynamically stable, transparent or translucent, and has an oil-droplet size of 0.005 to 0.5 μm. In some embodiments, the ophthalmic emulsion may include a cannabinoid, such as CBD, or derivatives thereof, an oil, an emulsifier, a surfactant (e.g., ionic or non-ionic surfactant), and water. In some embodiments, the mean droplet size is in the submicron range, e.g., between about 0.005 to 0.5 μm, alternatively between about 0.01 to 0.5 μm, alternatively between about 0.01 to 0.4 μm, alternatively between about 0.01 to 0.3 μm, alternatively between about 0.1 to 0.5 μm, alternatively between about 0.1 to 0.4 μm, alternatively between about 0.1 to 0.3 μm. In some embodiments, the ophthalmic emulsion may include oil in a proportion of 10-100,000 parts by weight, water in a proportion of 100-100,000 parts by weight and emulsifier in a proportion of 10-100,000 parts by weight, all per part by weight of a cannabinoid, such as CBD, or derivatives thereof; alternatively in the proportions of oil 10-10,000 parts by weight, water 100-50,000 parts by weight and emulsifier 10-10,000 parts by weight, all per part by weight of cannabinoid, such as CBD, or derivatives thereof; and alternatively in the proportions of oil 10-5,000 parts by weight, water 500-50,000 parts by weight and emulsifier 10-5,000 parts by weight, all per part by weight of cannabinoid, such as CBD, or derivatives thereof. The compositions described herein may be encapsulated in liposomes, micelles, noisome, fullerene, nanoshell, quantum dot, dendrimer, lipid-polymer nanoparticles, or any combination thereof. The compositions described herein can be coated on nanoparticles or charged polymers.

Emulsifiers may be incorporated in the ophthalmic formulation. For example, the formulation may include a phospholipid compound or a mixture of phospholipids. Suitable components include lecithin; EPICURON™ 120 (Lucas Meyer, Germany) which is a mixture of about 70% of phosphatidylcholine, 12% phosphatidylethanolamine and about 15% other phospholipids; OVOTHIN™ 160 (Lucas Meyer, Germany) which is a mixture comprising about 60% phosphatidylcholine, 18% phosphatidylethanolamine and 12% other phospholipids; a purified phospholipid mixture; LIPOID E-75 or LIPOID E-80 (Lipoid, Germany) which is a phospholipid mixture comprising about 80% phosphatidylcholine, 8% phosphatidylethanolamine, 3.6% non-polar lipids and about 2% sphingomyelin. Purified egg yolk phospholipids, soybean oil phospholipids or other purified phospholipid mixtures are useful as this component.

In some embodiments, the ophthalmic formulations may include micellar water. A therapeutically effective concentration of a cannabinoid and a bioactive agent herein described may be also included in the ophthalmic formulation that include micellar water.

### Punctal Plugs and Contact Lens

In some embodiments, the invention includes punctal plugs, contact lenses, and/or other ophthalmic inserts or devices that provide a sustained release of CBD to the eye. Punctal plugs are better suited for sustained release and controlled dose application compared to eye drops, solutions, and ointments because, for example, the application of eye drops tends to overfill the conjunctival sac, the pocket between the eye and the lids, causing a substantial portion of the drop to be lost due to overflow of the lid margin onto the cheek. In addition, a substantial portion of the drop remaining on the ocular surface is washed away by tears into the tear drainage system, thereby diluting the concentration of the drug. Contact lenses that are coated or embedded with a CBD-containing composition are well suited to release the composition when the lens is placed on an individual's eye.

Generally, a punctal plug includes a body portion sized to pass through a lacrimal punctum and be positioned within a lacrimal canaliculus of an eyelid. The active ingredient (CBD) may be impregnated within the material of the punctal plug (e.g., nanoparticles) or contained in an internal reservoir in fluid communication with the surface of the eye. Punctal plugs may be designed in accordance with the principles and features set forth in U.S. Pat. No. 6,196,993 which is hereby incorporated by reference in its entirety.

### Vaginal Formulations and Delivery Systems

Vaginal formulations may be prepared according to known principles and including established and pharmaceutically acceptable ingredients. Vaginal formulations include, but are not limited to, ointments, emulsions, gels, creams, inserts, capsules, and suppositories.

### Oleaginous Bases for Creams or Ointments

The vaginal formulation may include a cannabinoid, such as CBD, or derivatives thereof, in a hydrocarbon-based semisolid. The hydrocarbon-based semisolid can include a petrolatum component, such as paraffin waxes, mineral oil, mineral oil employing incorporated isobutylene, colloidal silica, colloidal silicon dioxide. Polysiloxanes, which are also known as silicones, are also suitable bases.

### Emulsion Bases for Creams or Ointments

Water-in-oil (W/O) emulsion bases can be prepared by taking a mixture of a cannabinoid with oil phase ingredients, bacteriostats/preservatives, and buffer salts that are dissolved or suspended therein and adding water to form a water-in-oil emulsion.

Oil-in-water emulsion (O/W) bases are semi-solid emulsions, microemulsions, or foam emulsions containing a cannabinoid. The internal oil phase may be about 10% to about 40% (e.g., about 11%, 12%, 13%, 14%, 15%, 16%, 17%, 18%, 19%, 20%, 21%, 22%, 23%, 24%, 25%, 26%, 27%, 28%, 29%, 30%, 31%, 32%, 32%, 33%, 34%, 35%, 36%, 37%, 38%, 39%, or 40%) oil by weight and the external phase may contain about 80% or more water. The oleaginous phase may contain long-chain alcohols (cetyl, stearyl), long-chain esters (myristates, palmitates, stearates), long-chain acids (palmitic, stearic), vegetable and animal oils and assorted waxes. The emulsion may include a surfactant, which may be anionic, cationic, nonionic, or amphoteric surfactants, or a combination, e.g., a combination of nonionic surfactants.

### Anhydrous Water-Soluble Bases for Creams or Ointments

Solutions or suspensions of a cannabinoid can be formulated with a buffer system in glycols can be used to make an anhydrous water-soluble base. The glycol may be glycerin, polyethylene glycol, propylene glycol. The solution or suspension may be thickened with a thickener, such as hydroxypropyl cellulose.

### Gels

Gels containing a cannabinoid can be formulated with gelling agents. Gelling agents include cationic polymers (e.g., polyquaternium-10), acrylate copolymers, alkyl celluloses, carboxyalkyl celluloses, carboxymethyl cellulose salts, guar gums, xanthan gum, hydroxyalkyl celluloses, poloxamers, polyvinyl alcohol, methyl vinyl ether/maleic anhydride (PVM/MA) copolymers, PVM/MA decadiene crosspolymers, carbomers (carboxyvinyl polymers), carbomer salts, acrylates/C10-30 alkyl acrylate crosspolymers, and hyaluronic acid. In some embodiments, the gelling agents are carbomers and acrylates/C10-30 alkyl acrylate crosspolymers.

### Vaginal Inserts and Suppositories

Suppositories containing a cannabinoid can be oleaginous in nature. The suppository is a solid but melts at body temperature. Suppositories containing a cannabinoid can include a polyethylene-glycol base, which dissolves in vaginal fluids. The suppository may have a solid outer layer (a lipoidal phase) that melts at body temperature. The suppository may also have a non-lipoidal internal phase that is an emulsion. The non-lipoidal internal phase may be miscible with water, and may contain water, glycerine, or combinations thereof. The non-lipoidal internal phase may be a solution, suspension, emulsion, or combination thereof, and contain the cannabinoid. The outer lipoidal phase includes components that have low solubility in water (or are insoluble in water) and are soluble in alcohol, ether, chloroform or other fat solvents. These outer lipoidal phase components include neutral fats, fatty acids, waxes, phosphatides, petrolatum, fatty acid esters of monoprotic alcohols, and mineral oils, and may optionally contain the cannabinoid. The suppository delivery system may be prepared as is well known in the art by mixing the internal phase with the external phase in a planetary-type mixer or a continuous mixer with multiple impellers.

### Vaginal Capsules

Soft gelatin capsules having a gelatin-based shell surrounding a liquid or solid fill may also be used to deliver the cannabinoid, such as CBD, or derivatives thereof, to the vaginal area to treat bacterial vaginosis. The soft gelatin may be made from a combination of gelatin, water, an opacifier, and a plasticizer, e.g., glycerin or sorbitol. The capsules may be filled with a therapeutically effective amount of a cannabinoid, such as CBD, or derivatives thereof and an excipient, such as a mono-unsaturated fatty acid excipient will facilitate its therapeutic use after being administered intravaginally. The contents of the capsule may be solid or liquid at room temperature, and may have a flow point, the temperature at which rapid flow of the sample occurs, in the range of about 30-40° C., alternatively about 30-37° C. The contents of the capsule may also contain additives, such as stabilizers (e.g., antioxidants and other types of preservatives), polymorphic transition accelerators (e.g., tristearin), biocompatible polymers, surfactants, dispersants, water absorbents and the like.

### Pessary

An intravaginal pessary is designed for arrangement in the vagina for compressive action on and to reduce protrusion of pelvic structures into the vagina. A pessary is inserted into the vagina to help support the bladder, vagina, uterus, and/or rectum. Typical pessary devices are large in diameter during use, and can elastically expand, inflate, or unfold to provide compressive action within the vagina. The pessary can be inserted into the vagina digitally or with an applicator. The pessary could contain, or be coated with, or otherwise provide delivery of the cannabinoid pharmaceutical composition.

### Ingredients of Vaginal Formulations

Any of the above-described vaginal formulations may include buffers. Suitable buffers include, for example, borates, borate-polyol complexes, succinate, phosphate buffering agents, citrate buffering agents, acetate buffering agents, carbonate buffering agents, organic buffering agents, amino acid buffering agents, or combinations thereof. The buffer should have the capacity to be in the range of pH 3-8. Examples of buffers include acetates such as sodium acetate; phosphates such as sodium dihydrogen phosphate, disodium hydrogen phosphate, potassium dihydrogen phosphate and dipotassium hydrogen phosphate; ε-aminocaproic acid; amino acid salts such as sodium glutamate; and boric acid and a salt thereof. The buffer is generally contained in a proportion of 0.01-2.0 w/v %, preferably 0.05-0.5 w/v % relative to the entire vaginal formulation. Other suitable buffers include, but are not limited to, sodium hydroxide, potassium hydroxide, lithium hydroxide, calcium hydroxide, sodium carbonate, sodium bicarbonate, potassium carbonate, potassium bicarbonate, and glucono-delta-lactone. Borates include boric acid, salts of boric acid, other pharmaceutically acceptable borates, and combinations thereof. In some cases, borates include boric acid, sodium borate, potassium borate, calcium borate, magnesium borate, manganese borate, and other such borate salts. Polyols include any compound having at least one hydroxyl group on each of two adjacent carbon atoms that are not in trans configuration relative to each other. In some embodiments, the polyols are linear or cyclic, substituted or unsubstituted, or mixtures thereof, so long as the resultant complex is water soluble and pharmaceutically acceptable. In some instances, examples of polyols include sugars, sugar alcohols, sugar acids and uronic acids. In some cases, polyols include, but are not limited to mannitol, glycerin, xylitol and sorbitol. Phosphate buffering agents include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; ammonium phosphates such as diammonium hydrogen phosphate and ammonium dihydrogen phosphate; or a combination thereof. In some instances, the phosphate buffering agent is an anhydride. In some instances, the phosphate buffering agent is a hydrate. Citrate buffering agents may include citric acid and sodium citrate. Acetate buffering agents include acetic acid, potassium acetate, and sodium acetate. Carbonate buffering agents may include sodium bicarbonate and sodium carbonate. Organic buffering agents may include Good's Buffer, such as for example 2-(N-morpholino)ethanesulfonic acid (MES), N-(2-Acetamido)iminodiacetic acid, N-(Carbamoylmethyl)iminodiacetic acid (ADA), piperazine-N,N′-bis(2-ethanesulfonic acid (PIPES), N-(2-acetamido)-2-aminoethanesulfonic acid (ACES), β-Hydroxy-4-morpholinepropanesulfonic acid, 3-Morpholino-2-hydroxypropanesulfonic acid (MOPSO), cholamine chloride, 3-(N-morpholino)propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), piperazine-1,4,-bis (2-hydroxypropanesulphonic acid) (POPSO), 4-(2-hydroxyethyl)piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; and diethanolamine (DEA). Amino acid buffering agents may include taurine, aspartic acid and its salts (e.g., potassium salts, etc.), E-aminocaproic acid, and the like.

Any of the above-described vaginal formulations may include thickeners. Thickeners include colloidal alumina, colloidal silica, alginic acid and derivatives thereof, “CARBOPOL®” (carboxyvinyl polymers), cellulose derivatives, such as “KLUCEL™” (cellulose ethers), “METHOCEL™” (methyl cellulose), “NATROSOL™” (hydroxyethyl cellulose), sodium carboxymethyl cellulose, gelatin, natural gums, such as agar, tragacanth, acacia gum, guar gum, stearates, isobutylene, waxes, carrageen, and the like, egg yolk, lecithin, pectin, THIXCIN®, resins like ethylene oxide polymers, such as the so called POLYOX™, and the like.

Any of the above-described vaginal formulations may include viscosity building agents. Viscosity building agents (viscosifiers) may be included to improve the characteristics of the formulation. Examples include non-ionic water-soluble polymers or other compounds that can lubricate, wet, or otherwise aid in the natural tear build-up. The compounds may enhance the viscosity of the formulation and include monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P. Other examples of viscosity building agents include, but are not limited to, polysaccharides, vinyl polymers, and acrylic acid polymers. Examples of polysaccharides include hyaluronic acid and its salts, chondroitin sulfate and its salts, dextrans, and various polymers of the cellulose family. Other examples of viscosity building agents include, but are not limited to, methyl cellulose, hydroxyethyl cellulose (HEC), ethyl hydroxyethyl cellulose, carboxymethyl cellulose and sodium carboxymethyl cellulose (Na CMC), starch derivatives such as moderately cross-linked starch, acrylic polymers such as carbomer and its derivatives (Polycarbophil, CARBOPOL®, etc.); polyethylene oxide (PEO), chitosan (poly-(D-glucosamine); natural polymers such as gelatin, sodium alginate, pectin, scleroglucan, tragacanth, gellan, xanthan gum or guar gum, poly co-(methyl vinyl ether/maleic anhydride), microcrystalline cellulose/AVICEL®), microcrystalline wax, and croscarmellose.

Any of the above-described vaginal formulations may include a pH adjusting agent. Suitable pH adjusting agents include hydrochloric acid, citric acid, phosphoric acid, acetic acid, tartaric acid, sodium hydroxide, potassium hydroxide, sodium carbonate and sodium hydrogencarbonate.

Any of the above-described vaginal formulations may include a preservative. Suitable preservatives include quaternary ammonium salts such as benzalkonium chloride, benzethonium chloride and the like; cationic compounds such as chlorhexidine gluconate and the like; p-hydroxybenzoates such as methyl p-hydroxybenzoate, propyl p-hydroxybenzoate and the like; alcohol compounds such as chlorobutanol, benzyl alcohol and the like; sodium dehydroacetate; thimerosal; sorbic acid; and the like. Other preservatives include ethyl, ethyl, propyl, and butyl esters of parahydroxybenzoic acid, propyl gallate, sorbic acid and its sodium and potassium salts, propionic acid and its calcium and sodium salts, “Dioxin” (6-acetoxy-2,4-dimethyl-m-dioxane), “Bronopol” (2-bromo-2-nitropropane-1,3-diol) and salicylanilides such as disbromosalicylanilide, tribromosalicylanilides, “CINARYL®” 100 and 200 or “DOWICIL™” 100 and 200 (Cis isomer of 1-(3-chloroallyl-3,5,7-triaza-1-azanidadamantane chloride), hexachlorophene, sodium benzoate, citric acid, ethylenediaminetetraacetic acid and its alkali metal and alkaline earth metal salts, butyl hydroxyanisole, butyl hydroxytoluene, phenolic compounds such as chloro- and bromocresols and chloro- and bromo-oxylenols, quaternary ammonium compounds like benzalkonium chloride, aromatic alcohols such as phenylethyl alcohol, benzyl alcohol, etc., chlorobutanol, quinoline derivatives such as iodochlorhydroxyquinoline, benzoic acid, and the like.

Any of the above-described vaginal formulations may include a penetration enhancer, which makes the cell membranes less rigid, and therefore, more amenable to allowing the drug to enter the cell. Suitable penetration enhancers include, but are not limited to, benzalkonium chloride and EDTA. Penetration enhancers may also be saccharide surfactants, such as dodecylmaltoside (DDM), and monoacyl phosphoglycerides, such as lysophosphatidylcholine. Penetration enhancers may also be a surfactant, bile salt or ethoxyglycol. Penetration enhancers may be present in an amount ranging from about 0.001 wt. % to about 3 wt. % (e.g., about 0.001 wt. % to about 0.01 wt. %, e.g., 0.002 wt. %, 0.003 wt. %, 0.004 wt. %, 0.005 wt. %, 0.006 wt. %, 0.007 wt. % 0.008 wt. %, 0.009 wt. %, or 0.01 wt. %, e.g., about 0.01 wt. % to about 0.1 wt. %, e.g., 0.02 wt. %, 0.03 wt. %, 0.04 wt. %, 0.05 wt. %, 0.06 wt. %, 0.07 wt. %, 0.08 wt. %, 0.09 wt. %, or 0.1 wt. %, e.g., about 0.1 wt. % to about 1.0 wt. %, e.g., 0.2 wt. %, 0.3 wt. %, 0.4 wt. %, 0.5 wt. %, 0.6 wt. %, 0.7 wt. %, 0.8 wt. %, 0.9 wt. %, or 1.0 wt. %, e.g., about 1.0 wt. % to about 3.0 wt. %, e.g., 1.5 wt. %, 2.0 wt. %, 2.5 wt. %, or 3.0 wt. %).

Any of the above-described vaginal formulations may include a surfactant. Suitable surfactants include, for example, polyoxyethylene fatty acid glycerides and vegetable oils, e.g., polyoxyethylene (60) hydrogenated castor oil; and polyoxyethylene alkyl ethers and alkylphenyl ethers, e.g., octoxynol-10 and octoxynol-40, phospholipids, cholesterol, and cholesterol fatty acid esters and derivatives thereof; nonionic surfactants including, for example, polyoxyethylene fatty alcohol esters, sorbitan fatty acid esters (Spans), polyoxyethylene sorbitan fatty acid esters (e.g., polyoxyethylene (20) sorbitan monooleate (TWEEN® 80), polyoxyethylene (20) sorbitan monostearate (TWEEN® 60), polyoxyethylene (20) sorbitan monolaurate (TWEEN® 20) and other Tweens, sorbitan esters, sorbitan tristearate, glycerol esters, e.g., Myrj and glycerol triacetate (triacetin), polyethylene glycols, cetyl alcohol, cetostearyl alcohol, stearyl alcohol, polysorbate 60, polysorbate 80, poloxamers, poloxamines, polyoxyethylene castor oil derivatives (e.g., CREMPHOR® RH40, CREMPHOR® A25, CREMPHOR® A20, CREMPHOR® EL) and other Cremphors, sulfosuccinates, alkyl sulphates (SLS); PEG glyceryl fatty acid esters such as PEG-8 glyceryl caprylate/caprate (LABRASOL®), PEG-4 glyceryl caprylate/caprate (LABRAFAC® Hydro WL 1219), PEG-32 glyceryl laurate (GELUCIRE® 444/14), PEG-6 glyceryl mono oleate (LABRAFIL® M 1944 CS), PEG-6 glyceryl linoleate (LABRAFIL® M 2125 CS); PEG-30 dipolyhydroxystearate; glyceryl monoisostearate, propylene glycol mono- and di-fatty acid esters, such as propylene glycol laurate, propylene glycol caprylate/caprate; BRIJ 700, Polyoxyethyen-20-monocetyl ether (BRIJ® 58); ascorbyl-6-palmitate, stearylamine, sodium lauryl sulfate, polyoxethylene glycerol triiricinoleate, and any combinations or mixtures thereof; anionic surfactants including, for example, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and cationic surfactants including, for example, cetyltrimethylammonium bromide, and lauryl dimethyl benzyl-ammonium chloride. Other surfactants include tyloxapol, PLURONIC™ F-68 (BASF, Ludwighsafen, Germany), and the poloxamer surfactants. Surfactants may be ionic (anionic or cationic), non-ionic, or ampholytic (having amino and carboxy groups). Surfactants in these categories include sorbitan trioleate, sorbitan tristearate, sorbitan sesquioleate, glycerol monostearate, sorbitan monostearate, sorbitan monopalmitate, sorbitan monolaurate, polyoxyethylene lauryl ether, polyethylene glycol 400 monostearate, triethanolamine oleate, polyoxyethylene glycol 400 monolaurate, polyoxyethylene sorbitan monostearate, polyoxyethylene sorbitan monooleate, polyoxyethylene sorbitan monolaurate, sodium oleate, potassium oleate, sodium lauryl sulfate, lauroyl imidazoline, sodium dodecylbenzene sulfonate, sodium monoglyceride sulfate, sodium alkaralkyl polyglycol sulfate, sodium oleyl taurate, sodium dioctyl sulfosuccinate, lauryl polyglycol, ether, sodium dibutyl naphthalene sulfonate, alkyl phenol polyglycol ether, sorbitan monolaurate polyglycol ether, sulfonated castor oil, tall oil polyglycol ester, alkyl dimethyl benzylammonium chloride, alkyl naphthalene pyridinium chloride, cetyl dimethyl ethylammonium bromide, alkyl dimethyl chlorobenzylammonium chloride, dibutyl phenyl phenol sulfonate, ester of colaminoethylformyl methylpyridinium chloride, sulfonated methyl oleylamide, sorbitan monolaurate polyglycol ether, polyglycol oleate, sodium lauryl sulfoacetate, sodium 2-ethylhexanol sulfate, sodium 7-ethyl-2-methylundecanol-4 sulfate, sodium 3,9-diethyltridecanol-6 sulfate, sodium lauryl and myristyl collamide sulfonate and N-(sodium sulfoethyl) oleamide, and the like. In one embodiment, the surfactant is non-ionic, e.g., polysorbate 80, to reduce irritation.

Any of the above-described vaginal formulations may include an oil. The oil may act as an emulsifier. Suitable oils include a medium chain triglyceride (MCT) oil (a triglyceride oil in which the carbohydrate chain has 8-12 carbons), a vegetable oil, a mineral oil, or mixtures thereof. MCT oil is available commercially, e.g., TCR® (trade name of Société Industrielle des Oléagineux, France for a mixture of triglycerides wherein about 95% of the fatty acid chains have 8 or 10 carbons) and MIGLYOL® 812 (trade name of Dynamit Nobel, Sweden for a mixed triester of glycerine and of caprylic and capric acids). Examples of vegetable oils include soybean oil, cotton seed oil, olive oil, sesame oil and castor oil. Other higher fatty acid glycerides such as peanut oil may be used. The mineral oils may be natural hydrocarbons or their synthetic analogs. Oily fatty acids, such as oleic acid and linoleic acid, fatty alcohols, such as oleyl alcohol, and fatty esters, such as sorbitan monooleate and sucrose mono-di- or tri-palmitate, can also be used as the oil component. Examples of triglycerides include WECOBEE® FS, which is derived from hydrogenated palm kernel vegetable oil, and WECOBEE® M, which is derived from fully hardened palm kernel oil.

Any of the above-described vaginal formulations may include an emulsifier. Emulsifiers include, but are not limited to, a phospholipid compound or a mixture of phospholipids. Suitable emulsifiers also include lecithin; EPIKURON™ 120 (Lucas Meyer, Germany) which is a mixture of about 70% of phosphatidylcholine, 12% phosphatidylethanolamine and about 15% other phospholipids; OVOTHIN™ 160 (Lucas Meyer, Germany) which is a mixture comprising about 60% phosphatidylcholine, 18% phosphatidylethanolamine and 12% other phospholipids; a purified phospholipid mixture; LIPOID E-75 or LIPOID E-80 (Lipoid, Germany) which is a phospholipid mixture comprising about 80% phosphatidylcholine, 8% phosphatidylethanolamine, 3.6% non-polar lipids and about 2% sphingomyelin. Purified egg yolk phospholipids, soybean oil phospholipids or other purified phospholipid mixtures are useful as this component. The ointment base may be a petrolatum and/or vaginally compatible oils including mineral oil, and other substances known in the art as being appropriate for intravaginal administration, such as polyethylene-mineral oil gel. The gelling polymer may be a sulphated polysaccharide or one of its derivatives, chitosan, linexolid, carbomer, and xanthan gum. Thermoreversible polymers include but are not limited to gelatin, poly(vinyl chloride), poly(acrylonitrile), polystyrene (atactic), poly(vinyl alcohol), agarose, carrageenans, benzohydroxamic acid, cellulose derivatives, poloxamer, polysaccharides, and hydroxyethyl cellulose.

### Lipid-Polymer Composite Particles

Lipid-polymer composite particles may be used to formulate a bioactive agent (e.g., a therapeutic agent, a nutraceutical agent, or a recreational agent, e.g., a cannabinoid) for delivery. Lipid-polymer composite particles include a defined complex of molecules (e.g., lipids and polymers) held together by noncovalent bonds, such as hydrogen bonds, Van der Waals forces, electrostatic interactions, hydrophobic effect, and Pi-Pi interactions. Lipid-polymer composite particles may include large complexes of molecules that form sphere-, rod-, or sheet-like structures. Lipid-polymer composite particles include, for example, micelles and LNPs. Lipid-polymer composite particles may have a predetermined size. The size of the structure may vary based on the components (e.g., size or number of molecules of the bioactive agent) packed within the structure.

The size of the lipid-polymer composite particle may vary from, e.g., about 10 nm to about 1,000 nm. Non-limiting examples of the Z-average mean particle diameters include, e.g., from about 10 nm to about 500 nm, from about 10 nm to about 100 nm, from about 500 nm to about 1000 nm. For example, the lipid-polymer composite particle may have a Z-average mean particle diameter of, e.g., about 10 nm, about 15 nm, bout 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, about 500 nm, about 505 nm, about 510 nm, about 515 nm, about 520 nm, about 525 nm, about 530 nm, about 535 nm, about 540 nm, about 545 nm, about 550 nm, about 555 nm, about 560 nm, about 565 nm, about 570 nm, about 575 nm, about 580 nm, about 585 nm, about 590 nm, about 595 nm, about 600 nm, about 605 nm, about 610 nm, about 615 nm, about 620 nm, about 625 nm, about 630 nm, about 635 nm, about 640 nm, about 645 nm, about 650 nm, about 655 nm, about 660 nm, about 665 nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, about 750 nm, about 755 nm, about 760 nm, about 765 nm, about 770 nm, about 775 nm, about 780 nm, about 785 nm, about 790 nm, about 795 nm, about 800 nm, about 805 nm, about 810 nm, about 815 nm, about 820 nm, about 825 nm, about 830 nm, about 835 nm, about 840 nm, about 845 nm, about 850 nm, about 855 nm, about 860 nm, about 865 nm, about 870 nm, about 875 nm, about 880 nm, about 885 nm, about 890 nm, about 895 nm, about 900 nm, about 905 nm, about 910 nm, about 915 nm, about 920 nm, about 925 nm, about 930 nm, about 935 nm, about 940 nm, about 945 nm, about 950 nm, about 955 nm, about 960 nm, about 965 nm, about 970 nm, about 975 nm, about 980 nm, about 985 nm, about 990 nm, about 995 nm, or about 1000 nm.

The mean particle diameter may be measured by zeta potential, dynamic light scattering (DLS), electrophoretic light scattering (ELS), static light scattering (SLS), molecular weight, electrophoretic mobility, size exclusion chromatography (SEC), field flow fractionation, or other methods known in the art. In particular embodiments, the lipid-polymer composite particle contains a Z-average mean particle diameter of from about 10 nm to about 100 nm. One of skill in the art would appreciate that a population of lipid-polymer composite particles (e.g., LNPs, or micelles) may have a range of Z-average mean particle diameters within the population. Thus, the population may be polydisperse. The population may have a polydispersity index of 0.3 or less (e.g., 0.05 to 0.3). The polydispersity index can be determined using DLS (see, e.g., ISO 22412:2017).

### Lipid Nanoparticles

Bioactive agents of the invention may be fully encapsulated in a lipid formulation, e.g., an LNP, or another lipid-polymer composite particle. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 2000/003683. LNPs may have a mean diameter of about 50 nm to about 150 nm, more typically about 60 nm to about 130 nm, more typically about 70 nm to about 110 nm, most typically about 70 nm to about 90 nm, and are substantially nontoxic. In addition, the bioactive agents when present in the LNPs of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to bioactive agent ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Non-limiting examples of cationic lipids include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can include, for example, from about 20 mol % to about 50 mol % or about 40 mol % (e.g., about 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, or 50 mol %) of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % (e.g., about 5 mol %, 10 mol %, 15 mol %, 20 mol %, 25 mol %, 30 mol %, 35 mol %, 40 mol %, 45 mol %, 50 mol %, 55 mol %, 60 mol %, 65 mol %, 70 mol %, 75 mol %, 80 mol %, 85 mol %, or 90 mol %) if cholesterol is included, of the total lipid present in the particle.

The conjugated lipid that inhibits aggregation of particles can be, for example, a polyethylene glycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C₁₈). The conjugated lipid that prevents aggregation of particles can be, for example, from 0 mol % to about 20 mol % or about 2 mol % (e.g., about 0.5 mol %, 0.6 mol %, 0.7 mol %, 0.8 mol %, 0.9 mol %, 1.0 mol %, 1.1 mol %, 1.2 mol %, 1.3 mol %, 1.4 mol %, 1.5 mol %, 1.6 mol %, 1.7 mol %, 1.8 mol %, 1.9 mol %, 2.0 mol %, or about 5.0 mol %, 10.0 mol %, 15.0 mol %, or 20.0 mol %) of the total lipid present in the particle.

The lipid can have a carbon chain of length of from 4 to 22 and a neutral, cationic, or anionic head group.

In some embodiments, the particle further includes a sterol, (e.g., cholesterol) at, e.g., about 10 mol % to about 60 mol % or about 50 mol % (e.g., about 15 mol %, 20 mol %, 25 mol %, 30 mol %, 35 mol %, 40 mol %, 45 mol %, 50 mol %, 55 mol %, or 60 mol %) of the total lipid present in the particle.

### Micelles

Micelles are a particular type of molecular assembly in which amphipathic molecules are arranged in a spherical structure such that all the hydrophobic portions of the molecules are directed inward, leaving the hydrophilic portions in contact with the surrounding aqueous phase. Micelles may be made of lipids. The micelle phase is caused by the packing behavior of single-tail lipids in a bilayer. The difficulty filling all the volume of the interior of a bilayer, while accommodating the area per head group forced on the molecule by the hydration of the lipid head group, leads to the formation of the micelle. This type of micelle is known as a normal-phase micelle (oil-in-water micelle). Inverse micelles have the head groups at the center with the tails extending out (water-in-oil micelle).

Micelles are approximately spherical in shape. Other phases, including shapes such as ellipsoids, cylinders, and bilayers, are also possible. The shape and size of a micelle are a function of the molecular geometry of its surfactant molecules and solution conditions such as surfactant concentration, temperature, pH, and ionic strength. The process of forming micelles is known as micellization and forms part of the phase behavior of many lipids according to their polymorphism.

### Phospholipids

The lipid-polymer composite particles described herein may include one or more phospholipids. Phospholipids generally consist of two hydrophobic fatty acid tails and a hydrophilic head with a phosphate group. The two components are usually joined together by a glycerol molecule. The phosphate groups can be modified with organic molecules such as choline, ethanolamine, or serine. Suitable phospholipids that may be used in the compositions described herein include, for example, phosphatidylcholine, phosphatidylserine, phosphatidylglycerol, phosphatidylethanolamine, phosphatidylinositol. The concentration of the phospholipid in the lipid-polymer compositive particles may be from about 2% to about 20% v/v (e.g., from about 4% to about 18%, from about 5% to about 15%, e.g., about 10%).

### Block Copolymers

The lipid-polymer composite particles described herein may include block copolymers. Block copolymers refer to a linear polymer having regions or blocks along its backbone chain that are characterized by similar hydrophilicity, hydrophobicity, or chemistry. Block copolymers may include, e.g., two, three, four, or more blocks (e.g., diblock or triblock copolymers). Multiblock copolymers include a plurality of blocks.

### Diblock Copolymers

The compositions described herein may include a diblock copolymer, which includes two distinct blocks of repeating polymer units. One example of a diblock copolymer as described herein includes an amphipathic copolymer, such as one with a region including a hydrophilic chain of repeated units connected to a region including a hydrophobic chain of repeating units with or without a linker. Such a diblock copolymer may include a hydrophilic chain of polyoxyethylene (PEO) subunits connected to a hydrophobic chain of polyoxypropylene (PPO) subunits. The diblock copolymer of PEO and PPO subunits can be represented by the following formula: X₁(C₂H₄O)ₘ-L-(C₃H₆O)ₙX₂. X₁ and X₂ may be any chemical moiety. L may be a linker that may optionally be present. In some embodiments, the PEO and PPO subunit blocks are directly covalently linked. In some embodiments, X₁ and X₂ are H and OH, respectively. Other diblock copolymers include, for example, poly(ethylene glycol)-poly(γ-benzyl L-glutamate) PEG-PBLA, poly(ethylene glycol)-poly(D,L-lactic acid) PEG-PDLLA, poly(ethylene glycol)-poly(L-lactic acid) PEG-PLLA, poly(ethylene glycol)-poly(ε-caprolactone) PEG-PCL, poly(ethylene glycol)-poly(D,L-lactide-co-glycolide) PEG-PLGA, poly(ethylene glycol)-poly (γ-benzyl L-glutamate) PEG-PBLG, poly(ethylene glycol)-poly(β-benzyl L-aspartate) PEG-PBLA, poly(ethylene glycol)-poly(α-benzyl carboxylate-ε-caprolactone) PEG-PBCL, and poly(ethylene glycol)-poly(δ-valerolactone) PEG-PVL. For clarity, as used herein, X₁-[PEO]-L-[PPO]-X₂ refers to a structure:

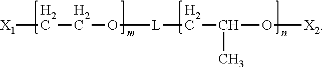

The lengths of the polymer blocks can be customized. As a result, many different diblock copolymers exist. Diblock copolymers suitable for use in conjunction with the compositions and methods of the present disclosure include those having an average molecular weight of from about 5 kDa to about 30 kDa (e.g., 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, or 30 kDa. Since the synthesis of diblock copolymers is associated with a natural degree of variation from one batch to another, the numerical values recited above (and those used herein to characterize a given diblock copolymer) may not be precisely achievable upon synthesis, and the average value will differ to a certain extent. Thus, the term “diblock copolymer” as used herein can be used interchangeably with the term “diblock copolymers” (representing an entity of several diblock copolymers, also referred to as mixture of diblock copolymers) if not explicitly stated otherwise. The term “average” in relation to the number of monomer units or molecular weight of (a) diblock copolymer(s) as used herein is a consequence of the technical inability to produce diblock copolymers all having the identical composition and thus the identical molecular weight. Diblock copolymers produced according to state-of-the-art methods will be present as a mixture of diblock copolymers each showing a variability as regards their molecular weight, but the mixture as a whole averaging the molecular weight specified herein.

### Poloxamers

One example of a triblock copolymer is a poloxamer. A poloxamer refers to a non-ionic triblock copolymer composed of a central hydrophobic chain of polyoxypropylene flanked by two hydrophilic chains of polyoxyethylene. Poloxamers are also known by the trade name of “PLURONIC®” or “SYNPERONIC®” (BASF). The block copolymer can be represented by the following formula: HO(C₂H₄O)ₓ(C₃H₆O)_{y}(C₂H₄O)_{z}H. The lengths of the polymer blocks can be customized. As a result, many different poloxamers exist. Since the synthesis of block copolymers is associated with a natural degree of variation from one batch to another, the numerical values used herein to characterize a given poloxamer may not be precisely achievable upon synthesis, and the average value will differ to a certain extent. Thus, the term “poloxamer” as used herein can be used interchangeably with the term “poloxamers” (representing an entity of several poloxamers, also referred to as mixture of poloxamers) if not explicitly stated otherwise. The term “average” in relation to the number of monomer units or molecular weight of (a) poloxamer(s) as used herein is a consequence of the technical inability to produce poloxamers all having the identical composition and thus the identical molecular weight. Poloxamers produced according to state-of-the-art methods will be present as a mixture of poloxamers each showing a variability as regards their molecular weight, but the mixture as a whole averaging the molecular weight specified herein. Poloxamers suitable for use with the compositions described herein are disclosed in Alexandridis and Bodratti, Journal of Functional Materials 9(1):11 (2018), the disclosure of which is incorporated herein by reference in its entirety.

Poloxamers that may be used in conjunction with the compositions and methods of the disclosure include those having an average molar mass of polyoxypropylene subunits of greater than 2,050 g/mol (e.g., an average molar mass of polyoxypropylene subunits of about 2,055 g/mol, 2,060 g/mol, 2,075 g/mol, 2,080 g/mol, 2,085 g/mol, 2,090 g/mol, 2,095 g/mol, 2,100 g/mol, 2,200 g/mol, 2,300 g/mol, 2,400 g/mol, 2,500 g/mol, 2,600 g/mol, 2,700 g/mol, 2,800 g/mol, 2,900 g/mol, 3,000 g/mol, 3,100 g/mol, 3,200 g/mol, 3,300 g/mol, 3,400 g/mol, 3,500 g/mol, 3,600 g/mol, 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of greater than 2,250 g/mol (e.g., an average molar mass of polyoxypropylene subunits of about 2,300 g/mol, 2,400 g/mol, 2,500 g/mol, 2,600 g/mol, 2,700 g/mol, 2,800 g/mol, 2,900 g/mol, 3,000 g/mol, 3,100 g/mol, 3,200 g/mol, 3,300 g/mol, 3,400 g/mol, 3,500 g/mol, 3,600 g/mol, 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of greater than 2,750 g/mol (e.g., an average molar mass of polyoxypropylene subunits of about 2,800 g/mol, 2,900 g/mol, 3,000 g/mol, 3,100 g/mol, 3,200 g/mol, 3,300 g/mol, 3,400 g/mol, 3,500 g/mol, 3,600 g/mol, 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of greater than 3,250 g/mol (e.g., an average molar mass of polyoxypropylene subunits of about 3,300 g/mol, 3,400 g/mol, 3,500 g/mol, 3,600 g/mol, 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of greater than 3,625 g/mol (e.g., an average molar mass of polyoxypropylene subunits of about 3,700 g/mol, 3,800 g/mol, 3,900 g/mol, 4,000 g/mol, 4,100 g/mol, 4,200 g/mol, 4,300 g/mol, 4,400 g/mol, 4,500 g/mol, 4,600 g/mol, 4,700 g/mol, 4,800 g/mol, 4,900 g/mol, or 5,000 g/mol).

In some embodiments, the poloxamer has an average molar mass of polyoxypropylene subunits of from about 2,050 g/mol to about 4,000 g/mol (e.g., about 2,050 g/mol, 2,055 g/mol, 2,060 g/mol, 2,065 g/mol, 2,070 g/mol, 2,075 g/mol, 2,080 g/mol, 2,085 g/mol, 2,090 g/mol, 2,095 g/mol, 2,100 g/mol, 2,105 g/mol, 2,110 g/mol, 2,115 g/mol, 2,120 g/mol, 2,125 g/mol, 2,130 g/mol, 2,135 g/mol, 2,140 g/mol, 2,145 g/mol, 2,150 g/mol, 2,155 g/mol, 2,160 g/mol, 2,165 g/mol, 2,170 g/mol, 2,175 g/mol, 2,180 g/mol, 2,185 g/mol, 2,190 g/mol, 2,195 g/mol, 2,200 g/mol, 2,205 g/mol, 2,210 g/mol, 2,215 g/mol, 2,220 g/mol, 2,225 g/mol, 2,230 g/mol, 2,235 g/mol, 2,240 g/mol, 2,245 g/mol, 2,250 g/mol, 2,255 g/mol, 2,260 g/mol, 2,265 g/mol, 2,270 g/mol, 2,275 g/mol, 2,280 g/mol, 2,285 g/mol, 2,290 g/mol, 2,295 g/mol, 2,300 g/mol, 2,305 g/mol, 2,310 g/mol, 2,315 g/mol, 2,320 g/mol, 2,325 g/mol, 2,330 g/mol, 2,335 g/mol, 2,340 g/mol, 2,345 g/mol, 2,350 g/mol, 2,355 g/mol, 2,360 g/mol, 2,365 g/mol, 2,370 g/mol, 2,375 g/mol, 2,380 g/mol, 2,385 g/mol, 2,390 g/mol, 2,395 g/mol, 2,400 g/mol, 2,405 g/mol, 2,410 g/mol, 2,415 g/mol, 2,420 g/mol, 2,425 g/mol, 2,430 g/mol, 2,435 g/mol, 2,440 g/mol, 2,445 g/mol, 2,450 g/mol, 2,455 g/mol, 2,460 g/mol, 2,465 g/mol, 2,470 g/mol, 2,475 g/mol, 2,480 g/mol, 2,485 g/mol, 2,490 g/mol, 2,495 g/mol, 2,500 g/mol, 2,505 g/mol, 2,510 g/mol, 2,515 g/mol, 2,520 g/mol, 2,525 g/mol, 2,530 g/mol, 2,535 g/mol, 2,540 g/mol, 2,545 g/mol, 2,550 g/mol, 2,555 g/mol, 2,560 g/mol, 2,565 g/mol, 2,570 g/mol, 2,575 g/mol, 2,580 g/mol, 2,585 g/mol, 2,590 g/mol, 2,595 g/mol, 2,600 g/mol, 2,605 g/mol, 2,610 g/mol, 2,615 g/mol, 2,620 g/mol, 2,625 g/mol, 2,630 g/mol, 2,635 g/mol, 2,640 g/mol, 2,645 g/mol, 2,650 g/mol, 2,655 g/mol, 2,660 g/mol, 2,665 g/mol, 2,670 g/mol, 2,675 g/mol, 2,680 g/mol, 2,685 g/mol, 2,690 g/mol, 2,695 g/mol, 2,700 g/mol, 2,705 g/mol, 2,710 g/mol, 2,715 g/mol, 2,720 g/mol, 2,725 g/mol, 2,730 g/mol, 2,735 g/mol, 2,740 g/mol, 2,745 g/mol, 2,750 g/mol, 2,755 g/mol, 2,760 g/mol, 2,765 g/mol, 2,770 g/mol, 2,775 g/mol, 2,780 g/mol, 2,785 g/mol, 2,790 g/mol, 2,795 g/mol, 2,800 g/mol, 2,805 g/mol, 2,810 g/mol, 2,815 g/mol, 2,820 g/mol, 2,825 g/mol, 2,830 g/mol, 2,835 g/mol, 2,840 g/mol, 2,845 g/mol, 2,850 g/mol, 2,855 g/mol, 2,860 g/mol, 2,865 g/mol, 2,870 g/mol, 2,875 g/mol, 2,880 g/mol, 2,885 g/mol, 2,890 g/mol, 2,895 g/mol, 2,900 g/mol, 2,905 g/mol, 2,910 g/mol, 2,915 g/mol, 2,920 g/mol, 2,925 g/mol, 2,930 g/mol, 2,935 g/mol, 2,940 g/mol, 2,945 g/mol, 2,950 g/mol, 2,955 g/mol, 2,960 g/mol, 2,965 g/mol, 2,970 g/mol, 2,975 g/mol, 2,980 g/mol, 2,985 g/mol, 2,990 g/mol, 2,995 g/mol, 3,000 g/mol, 3,005 g/mol, 3,010 g/mol, 3,015 g/mol, 3,020 g/mol, 3,025 g/mol, 3,030 g/mol, 3,035 g/mol, 3,040 g/mol, 3,045 g/mol, 3,050 g/mol, 3,055 g/mol, 3,060 g/mol, 3,065 g/mol, 3,070 g/mol, 3,075 g/mol, 3,080 g/mol, 3,085 g/mol, 3,090 g/mol, 3,095 g/mol, 3,100 g/mol, 3,105 g/mol, 3,110 g/mol, 3,115 g/mol, 3,120 g/mol, 3,125 g/mol, 3,130 g/mol, 3,135 g/mol, 3,140 g/mol, 3,145 g/mol, 3,150 g/mol, 3,155 g/mol, 3,160 g/mol, 3,165 g/mol, 3,170 g/mol, 3,175 g/mol, 3,180 g/mol, 3,185 g/mol, 3,190 g/mol, 3,195 g/mol, 3,200 g/mol, 3,205 g/mol, 3,210 g/mol, 3,215 g/mol, 3,220 g/mol, 3,225 g/mol, 3,230 g/mol, 3,235 g/mol, 3,240 g/mol, 3,245 g/mol, 3,250 g/mol, 3,255 g/mol, 3,260 g/mol, 3,265 g/mol, 3,270 g/mol, 3,275 g/mol, 3,280 g/mol, 3,285 g/mol, 3,290 g/mol, 3,295 g/mol, 3,300 g/mol, 3,305 g/mol, 3,310 g/mol, 3,315 g/mol, 3,320 g/mol, 3,325 g/mol, 3,330 g/mol, 3,335 g/mol, 3,340 g/mol, 3,345 g/mol, 3,350 g/mol, 3,355 g/mol, 3,360 g/mol, 3,365 g/mol, 3,370 g/mol, 3,375 g/mol, 3,380 g/mol, 3,385 g/mol, 3,390 g/mol, 3,395 g/mol, 3,400 g/mol, 3,405 g/mol, 3,410 g/mol, 3,415 g/mol, 3,420 g/mol, 3,425 g/mol, 3,430 g/mol, 3,435 g/mol, 3,440 g/mol, 3,445 g/mol, 3,450 g/mol, 3,455 g/mol, 3,460 g/mol, 3,465 g/mol, 3,470 g/mol, 3,475 g/mol, 3,480 g/mol, 3,485 g/mol, 3,490 g/mol, 3,495 g/mol, 3,500 g/mol, 3,505 g/mol, 3,510 g/mol, 3,515 g/mol, 3,520 g/mol, 3,525 g/mol, 3,530 g/mol, 3,535 g/mol, 3,540 g/mol, 3,545 g/mol, 3,550 g/mol, 3,555 g/mol, 3,560 g/mol, 3,565 g/mol, 3,570 g/mol, 3,575 g/mol, 3,580 g/mol, 3,585 g/mol, 3,590 g/mol, 3,595 g/mol, 3,600 g/mol, 3,605 g/mol, 3,610 g/mol, 3,615 g/mol, 3,620 g/mol, 3,625 g/mol, 3,630 g/mol, 3,635 g/mol, 3,640 g/mol, 3,645 g/mol, 3,650 g/mol, 3,655 g/mol, 3,660 g/mol, 3,665 g/mol, 3,670 g/mol, 3,675 g/mol, 3,680 g/mol, 3,685 g/mol, 3,690 g/mol, 3,695 g/mol, 3,700 g/mol, 3,705 g/mol, 3,710 g/mol, 3,715 g/mol, 3,720 g/mol, 3,725 g/mol, 3,730 g/mol, 3,735 g/mol, 3,740 g/mol, 3,745 g/mol, 3,750 g/mol, 3,755 g/mol, 3,760 g/mol, 3,765 g/mol, 3,770 g/mol, 3,775 g/mol, 3,780 g/mol, 3,785 g/mol, 3,790 g/mol, 3,795 g/mol, 3,800 g/mol, 3,805 g/mol, 3,810 g/mol, 3,815 g/mol, 3,820 g/mol, 3,825 g/mol, 3,830 g/mol, 3,835 g/mol, 3,840 g/mol, 3,845 g/mol, 3,850 g/mol, 3,855 g/mol, 3,860 g/mol, 3,865 g/mol, 3,870 g/mol, 3,875 g/mol, 3,880 g/mol, 3,885 g/mol, 3,890 g/mol, 3,895 g/mol, 3,900 g/mol, 3,905 g/mol, 3,910 g/mol, 3,915 g/mol, 3,920 g/mol, 3,925 g/mol, 3,930 g/mol, 3,935 g/mol, 3,940 g/mol, 3,945 g/mol, 3,950 g/mol, 3,955 g/mol, 3,960 g/mol, 3,965 g/mol, 3,970 g/mol, 3,975 g/mol, 3,980 g/mol, 3,985 g/mol, 3,990 g/mol, 3,995 g/mol, or 4,000 g/mol).

In some embodiments, the poloxamer has an average molar mass of polyoxypropylene subunits of from about 2,750 g/mol to about 4,000 g/mol (e.g., about 2,750 g/mol, 2,755 g/mol, 2,760 g/mol, 2,765 g/mol, 2,770 g/mol, 2,775 g/mol, 2,780 g/mol, 2,785 g/mol, 2,790 g/mol, 2,795 g/mol, 2,800 g/mol, 2,805 g/mol, 2,810 g/mol, 2,815 g/mol, 2,820 g/mol, 2,825 g/mol, 2,830 g/mol, 2,835 g/mol, 2,840 g/mol, 2,845 g/mol, 2,850 g/mol, 2,855 g/mol, 2,860 g/mol, 2,865 g/mol, 2,870 g/mol, 2,875 g/mol, 2,880 g/mol, 2,885 g/mol, 2,890 g/mol, 2,895 g/mol, 2,900 g/mol, 2,905 g/mol, 2,910 g/mol, 2,915 g/mol, 2,920 g/mol, 2,925 g/mol, 2,930 g/mol, 2,935 g/mol, 2,940 g/mol, 2,945 g/mol, 2,950 g/mol, 2,955 g/mol, 2,960 g/mol, 2,965 g/mol, 2,970 g/mol, 2,975 g/mol, 2,980 g/mol, 2,985 g/mol, 2,990 g/mol, 2,995 g/mol, 3,000 g/mol, 3,005 g/mol, 3,010 g/mol, 3,015 g/mol, 3,020 g/mol, 3,025 g/mol, 3,030 g/mol, 3,035 g/mol, 3,040 g/mol, 3,045 g/mol, 3,050 g/mol, 3,055 g/mol, 3,060 g/mol, 3,065 g/mol, 3,070 g/mol, 3,075 g/mol, 3,080 g/mol, 3,085 g/mol, 3,090 g/mol, 3,095 g/mol, 3,100 g/mol, 3,105 g/mol, 3,110 g/mol, 3,115 g/mol, 3,120 g/mol, 3,125 g/mol, 3,130 g/mol, 3,135 g/mol, 3,140 g/mol, 3,145 g/mol, 3,150 g/mol, 3,155 g/mol, 3,160 g/mol, 3,165 g/mol, 3,170 g/mol, 3,175 g/mol, 3,180 g/mol, 3,185 g/mol, 3,190 g/mol, 3,195 g/mol, 3,200 g/mol, 3,205 g/mol, 3,210 g/mol, 3,215 g/mol, 3,220 g/mol, 3,225 g/mol, 3,230 g/mol, 3,235 g/mol, 3,240 g/mol, 3,245 g/mol, 3,250 g/mol, 3,255 g/mol, 3,260 g/mol, 3,265 g/mol, 3,270 g/mol, 3,275 g/mol, 3,280 g/mol, 3,285 g/mol, 3,290 g/mol, 3,295 g/mol, 3,300 g/mol, 3,305 g/mol, 3,310 g/mol, 3,315 g/mol, 3,320 g/mol, 3,325 g/mol, 3,330 g/mol, 3,335 g/mol, 3,340 g/mol, 3,345 g/mol, 3,350 g/mol, 3,355 g/mol, 3,360 g/mol, 3,365 g/mol, 3,370 g/mol, 3,375 g/mol, 3,380 g/mol, 3,385 g/mol, 3,390 g/mol, 3,395 g/mol, 3,400 g/mol, 3,405 g/mol, 3,410 g/mol, 3,415 g/mol, 3,420 g/mol, 3,425 g/mol, 3,430 g/mol, 3,435 g/mol, 3,440 g/mol, 3,445 g/mol, 3,450 g/mol, 3,455 g/mol, 3,460 g/mol, 3,465 g/mol, 3,470 g/mol, 3,475 g/mol, 3,480 g/mol, 3,485 g/mol, 3,490 g/mol, 3,495 g/mol, 3,500 g/mol, 3,505 g/mol, 3,510 g/mol, 3,515 g/mol, 3,520 g/mol, 3,525 g/mol, 3,530 g/mol, 3,535 g/mol, 3,540 g/mol, 3,545 g/mol, 3,550 g/mol, 3,555 g/mol, 3,560 g/mol, 3,565 g/mol, 3,570 g/mol, 3,575 g/mol, 3,580 g/mol, 3,585 g/mol, 3,590 g/mol, 3,595 g/mol, 3,600 g/mol, 3,605 g/mol, 3,610 g/mol, 3,615 g/mol, 3,620 g/mol, 3,625 g/mol, 3,630 g/mol, 3,635 g/mol, 3,640 g/mol, 3,645 g/mol, 3,650 g/mol, 3,655 g/mol, 3,660 g/mol, 3,665 g/mol, 3,670 g/mol, 3,675 g/mol, 3,680 g/mol, 3,685 g/mol, 3,690 g/mol, 3,695 g/mol, 3,700 g/mol, 3,705 g/mol, 3,710 g/mol, 3,715 g/mol, 3,720 g/mol, 3,725 g/mol, 3,730 g/mol, 3,735 g/mol, 3,740 g/mol, 3,745 g/mol, 3,750 g/mol, 3,755 g/mol, 3,760 g/mol, 3,765 g/mol, 3,770 g/mol, 3,775 g/mol, 3,780 g/mol, 3,785 g/mol, 3,790 g/mol, 3,795 g/mol, 3,800 g/mol, 3,805 g/mol, 3,810 g/mol, 3,815 g/mol, 3,820 g/mol, 3,825 g/mol, 3,830 g/mol, 3,835 g/mol, 3,840 g/mol, 3,845 g/mol, 3,850 g/mol, 3,855 g/mol, 3,860 g/mol, 3,865 g/mol, 3,870 g/mol, 3,875 g/mol, 3,880 g/mol, 3,885 g/mol, 3,890 g/mol, 3,895 g/mol, 3,900 g/mol, 3,905 g/mol, 3,910 g/mol, 3,915 g/mol, 3,920 g/mol, 3,925 g/mol, 3,930 g/mol, 3,935 g/mol, 3,940 g/mol, 3,945 g/mol, 3,950 g/mol, 3,955 g/mol, 3,960 g/mol, 3,965 g/mol, 3,970 g/mol, 3,975 g/mol, 3,980 g/mol, 3,985 g/mol, 3,990 g/mol, 3,995 g/mol, or 4,000 g/mol).

In some embodiments, the poloxamer has an average molar mass of polyoxypropylene subunits of from about 3,250 g/mol to about 4,000 g/mol (e.g., about 3,250 g/mol, 3,255 g/mol, 3,260 g/mol, 3,265 g/mol, 3,270 g/mol, 3,275 g/mol, 3,280 g/mol, 3,285 g/mol, 3,290 g/mol, 3,295 g/mol, 3,300 g/mol, 3,305 g/mol, 3,310 g/mol, 3,315 g/mol, 3,320 g/mol, 3,325 g/mol, 3,330 g/mol, 3,335 g/mol, 3,340 g/mol, 3,345 g/mol, 3,350 g/mol, 3,355 g/mol, 3,360 g/mol, 3,365 g/mol, 3,370 g/mol, 3,375 g/mol, 3,380 g/mol, 3,385 g/mol, 3,390 g/mol, 3,395 g/mol, 3,400 g/mol, 3,405 g/mol, 3,410 g/mol, 3,415 g/mol, 3,420 g/mol, 3,425 g/mol, 3,430 g/mol, 3,435 g/mol, 3,440 g/mol, 3,445 g/mol, 3,450 g/mol, 3,455 g/mol, 3,460 g/mol, 3,465 g/mol, 3,470 g/mol, 3,475 g/mol, 3,480 g/mol, 3,485 g/mol, 3,490 g/mol, 3,495 g/mol, 3,500 g/mol, 3,505 g/mol, 3,510 g/mol, 3,515 g/mol, 3,520 g/mol, 3,525 g/mol, 3,530 g/mol, 3,535 g/mol, 3,540 g/mol, 3,545 g/mol, 3,550 g/mol, 3,555 g/mol, 3,560 g/mol, 3,565 g/mol, 3,570 g/mol, 3,575 g/mol, 3,580 g/mol, 3,585 g/mol, 3,590 g/mol, 3,595 g/mol, 3,600 g/mol, 3,605 g/mol, 3,610 g/mol, 3,615 g/mol, 3,620 g/mol, 3,625 g/mol, 3,630 g/mol, 3,635 g/mol, 3,640 g/mol, 3,645 g/mol, 3,650 g/mol, 3,655 g/mol, 3,660 g/mol, 3,665 g/mol, 3,670 g/mol, 3,675 g/mol, 3,680 g/mol, 3,685 g/mol, 3,690 g/mol, 3,695 g/mol, 3,700 g/mol, 3,705 g/mol, 3,710 g/mol, 3,715 g/mol, 3,720 g/mol, 3,725 g/mol, 3,730 g/mol, 3,735 g/mol, 3,740 g/mol, 3,745 g/mol, 3,750 g/mol, 3,755 g/mol, 3,760 g/mol, 3,765 g/mol, 3,770 g/mol, 3,775 g/mol, 3,780 g/mol, 3,785 g/mol, 3,790 g/mol, 3,795 g/mol, 3,800 g/mol, 3,805 g/mol, 3,810 g/mol, 3,815 g/mol, 3,820 g/mol, 3,825 g/mol, 3,830 g/mol, 3,835 g/mol, 3,840 g/mol, 3,845 g/mol, 3,850 g/mol, 3,855 g/mol, 3,860 g/mol, 3,865 g/mol, 3,870 g/mol, 3,875 g/mol, 3,880 g/mol, 3,885 g/mol, 3,890 g/mol, 3,895 g/mol, 3,900 g/mol, 3,905 g/mol, 3,910 g/mol, 3,915 g/mol, 3,920 g/mol, 3,925 g/mol, 3,930 g/mol, 3,935 g/mol, 3,940 g/mol, 3,945 g/mol, 3,950 g/mol, 3,955 g/mol, 3,960 g/mol, 3,965 g/mol, 3,970 g/mol, 3,975 g/mol, 3,980 g/mol, 3,985 g/mol, 3,990 g/mol, 3,995 g/mol, or 4,000 g/mol).

In some embodiments, the poloxamer has an average molar mass of polyoxypropylene subunits of from about 3,625 g/mol to about 4,000 g/mol (e.g., about 3,625 g/mol, 3,630 g/mol, 3,635 g/mol, 3,640 g/mol, 3,645 g/mol, 3,650 g/mol, 3,655 g/mol, 3,660 g/mol, 3,665 g/mol, 3,670 g/mol, 3,675 g/mol, 3,680 g/mol, 3,685 g/mol, 3,690 g/mol, 3,695 g/mol, 3,700 g/mol, 3,705 g/mol, 3,710 g/mol, 3,715 g/mol, 3,720 g/mol, 3,725 g/mol, 3,730 g/mol, 3,735 g/mol, 3,740 g/mol, 3,745 g/mol, 3,750 g/mol, 3,755 g/mol, 3,760 g/mol, 3,765 g/mol, 3,770 g/mol, 3,775 g/mol, 3,780 g/mol, 3,785 g/mol, 3,790 g/mol, 3,795 g/mol, 3,800 g/mol, 3,805 g/mol, 3,810 g/mol, 3,815 g/mol, 3,820 g/mol, 3,825 g/mol, 3,830 g/mol, 3,835 g/mol, 3,840 g/mol, 3,845 g/mol, 3,850 g/mol, 3,855 g/mol, 3,860 g/mol, 3,865 g/mol, 3,870 g/mol, 3,875 g/mol, 3,880 g/mol, 3,885 g/mol, 3,890 g/mol, 3,895 g/mol, 3,900 g/mol, 3,905 g/mol, 3,910 g/mol, 3,915 g/mol, 3,920 g/mol, 3,925 g/mol, 3,930 g/mol, 3,935 g/mol, 3,940 g/mol, 3,945 g/mol, 3,950 g/mol, 3,955 g/mol, 3,960 g/mol, 3,965 g/mol, 3,970 g/mol, 3,975 g/mol, 3,980 g/mol, 3,985 g/mol, 3,990 g g/mol, 3995 g/mol, or 4,000 g/mol).

In some embodiments, the poloxamer has an average ethylene oxide content of greater than 40% by mass (e.g., about 41%, 42%, 43% m 44% 45%, 46%, 47%, 48%, 49%, 50%, 51%, 52%, 53%, 54%, 55%, 56%, 57%, 58%, 59%, 60%, 61%, 62% 63%, 64%, 65,%, 66%, 67%, 68%, 69%, 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the poloxamer has an average ethylene content of greater than 50% by mass (e.g., about 50%, 51%, 52%, 53%, 54%, 55%, 56% 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the poloxamer has an average ethylene oxide content of greater than 60% b mass (e.g., about 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, or more).

In some embodiments, the poloxamer has an average ethylene oxide content of greater than 70% by mass (e.g., about 60%, 61%, 62%, 63%, 64%, 65%, 66%, 67%, 68%, 69%, 70%, 71%, 72%, 73%, 74%, 75%, 76%, 77% 78%, 79%, 80%, 81%, 82%, 83%, 84%, 85%, 86%, 87%, 88%, 89%, 90%, 91%, 92%, 93%, 94%, 95%, or more).

In some embodiments, the poloxamer has an average ethylene oxide content of from about 40% to about 90% (e.g., about 40%, 41%, 42%, 43%, 44%, 45%, 46%, 47%, 48%, 49%, 50%, 51%, 52%, 53%, 54%, 55%, 56%, 57%, 58%, 59% 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%).

In some embodiments, the poloxamer has an average ethylene oxide content of from about 50% to about 85% (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, or 85%).

In some embodiments, the poloxamer has an average ethylene oxide content of from about 60% to about 80% (e.g., about 60%, 61%, 62%, 63%, 64%, 65%, 66%, 67%, 68%, 69%, 70%, 71%, 72%, 73%, 74%, 75%, 76%, 77%, 78%, 79%, 80%).

In some embodiments, the poloxamer has an average molar mass of greater than 10,000 g/mol (e.g., about 10,100 g/mol, 10,200 g/mol, 10,300 g/mol, 10,400 g/mol, 10,500 g/mol, 1,600 g/mol, 10,700 g/mol, 10,800 g/mol, 10,900 g/mol, 11,000 g/mol, 11,100 g/mol, 11,900 g/mol, 11,300 g/mol, 11,400 g/mol, 11,500 g/mol, 11,600 g/mol, 12,400 g/mol, 12,500 g/mol, 12,600 g/mol, 12,700 g/mol, 12,100 g/mol, 12,200 g/mol, 12,300 g/m g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of greater than 11,000 g/mol (e.g., about 11,100 g/mol, 11,200 g/mol, 11,300 g/mol, 11,400 g/mol, 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of greater than 12,000 g/mol (e.g., about 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of greater than 12,500 g/mol (e.g., about 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 10,000 g/mol to about 15,000 g/mol (e.g., about 10,000 g/mol, 10,100 g/mol, 10,200 g/mol, 10,300 g/mol, 10,400 g/mol, 10,500 g/mol, 10,600 g/mol, 10,700 g/mol, 10,800 g/mol, 10,900 g/mol, 11,000 g/mol, 11,100 g/mol, 11,200 g/mol, 11,300 g/mol, 11,400 g/mol, 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 11,000 g/mol to about 15,000 g/mol (e.g., about 11,000 g/mol, 11,100 g/mol, 11,200 g/mol, 11,300 g/mol, 11,400 g/mol, 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 11,500 g/mol to about 15,000 g/mol (e.g., about 11,500 g/mol, 11,600 g/mol, 11,700 g/mol, 11,800 g/mol, 11,900 g/mol, 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 12,000 g/mol to about 15,000 g/mol (e.g., about 12,000 g/mol, 12,100 g/mol, 12,200 g/mol, 12,300 g/mol, 12,400 g/mol, 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

In some embodiments, the poloxamer has an average molar mass of from about 12,500 g/mol to about 15,000 g/mol (e.g., about 12,500 g/mol, 12,600 g/mol, 12,700 g/mol, 12,800 g/mol, 12,900 g/mol, 13,000 g/mol, 13,100 g/mol, 13,200 g/mol, 13,300 g/mol, 13,400 g/mol, 13,500 g/mol, 13,600 g/mol, 13,700 g/mol, 13,800 g/mol, 13,900 g/mol, 14,000 g/mol, 14,100 g/mol, 14,200 g/mol, 14,300 g/mol, 14,400 g/mol, 14,500 g/mol, 14,600 g/mol, 14,700 g/mol, 14,800 g/mol, 14,900 g/mol, or 15,000 g/mol).

Poloxamers P288, P335, P338, and P407

Poloxamers that may be used in conjunction with the compositions and methods of the disclosure include “poloxamer 288” (also referred to in the art as “P288” and poloxamer “F98”) having the approximate chemical formula HO(C₂H₄O)ₓ(C₃H₆O)_{y}(C₂H₄O)_{z}H, wherein the sum of x and y is about 236.36, and z is about 44.83. The average molecular weight of P288 is about 13,000 g/mol.

In some embodiments, the poloxamer is a variant of P288, such as a variant of the formula HO(C₂H₄O)ₓ(C₃H₆O)_{y}(C₂H₄O)_{z}H, wherein the sum of x and y is from about 220 to about 250, and z is from about 40 to about 50. In some embodiments, the average molecular weight of the poloxamer is from about 12,000 g/mol to about 14,000 g/mol.

Poloxamers that may be used in conjunction with the compositions and methods of the disclosure further include “poloxamer 335” (also referred to in the art as “P335” and poloxamer “P105”), having the approximate chemical formula HO(C₂H₄O)ₓ(C₃H₆O)_{y}(C₂H₄O)_{z}H, wherein the sum of x and y is about 73.86, and z is about 56.03. The average molecular weight of P335 is about 6,500 g/mol.

In some embodiments, the poloxamer is a variant of P335, such as a variant of the formula HO(C₂H₄O)ₓ(C₃H₆O)_{y}(C₂H₄O)_{z}H, wherein the sum of x and y is from about 60 to about 80, and z is from about 50 to about 60. In some embodiments, the average molecular weight of the poloxamer is from about 6,000 g/mol to about 7,000 g/mol.

Poloxamers that may be used in conjunction with the compositions and methods of the disclosure further include “poloxamer 338” (also referred to in the art as “P338” and poloxamer “F108”), having the approximate chemical formula HO(C₂H₄O)ₓ(C₃H₆O)_{y}(C₂H₄O)_{z}H, wherein the sum of x and y is about 265.45, and z is about 50.34. The average molecular weight of P335 is about 14,600 g/mol.

In some embodiments, the poloxamer is a variant of P338, such as a variant of the formula HO(C₂H₄O)ₓ(C₃H₆O)_{y}(C₂H₄O)_{z}H, wherein the sum of x and y is from about 260 to about 270, and z is from about 45 to about 55. In some embodiments, the average molecular weight of the poloxamer is from about 14,000 g/mol to about 15,000 g/mol.

Poloxamers that may be used in conjunction with the compositions and methods of the disclosure further include “poloxamer 407” (also referred to in the art as “P407” and poloxamer “F127”), having the approximate chemical formula HO(C₂H₄O)ₓ(C₃H₆O)_{y}(C₂H₄O)_{z}H, wherein the sum of x and y is about 200.45, and z is about 65.17. The average molecular weight is about 12,600 g/mol.

In some embodiments, the poloxamer is a variant of P407, such as a variant of the formula HO(C₂H₄O)ₓ(C₃H₆O)_{y}(C₂H₄O)_{z}H, wherein the sum of x and y is from about 190 to about 210, and z is from about 60 to about 70. In some embodiments, the average molecular weight of the poloxamer is from about 12,000 g/mol to about 13,000 g/mol.

For clarity, the terms “average molar mass” and “average molecular weight” are used interchangeable herein to refer to the same quantity. The average molar mass, ethylene oxide content, and propylene oxide content of a poloxamer, as described herein, can be determined using methods disclosed in Alexandridis and Hatton, Colloids and Surfaces A: Physicochemical and Engineering Aspects 96:1-46 (1995), the disclosure of which is incorporated herein by reference in its entirety.

### Sterols

The lipid-polymer composite particles described herein may further include one or more sterols. Sterols are lipids that are often found naturally in plants, animals, and fungi. Phytosterols refers to a class of plant sterol molecules, which are naturally occurring compounds found in plant cell membranes. Phytosterols include both plant sterols and stanols. Phytosterols may be derived from any common plant source, such as soy, wood, tall oil, vegetable oil, and the like. Phytosterols include β-sitosterol, campesterol, stigmasterol, stigmastanol, campestanol, brassicasterol, ergosterol, lupeol, cycloartenol, and the like. The sterol as described herein may be any cholesterol or derivative thereof that alters the fluidity of a lipid layer. The sterol may be a naturally occurring sterol, e.g., a sterol derived or found in a natural source. Alternatively, the sterol may be a synthetic sterol, e.g., a sterol analog or derivative that is not naturally existing. In some preferred embodiments, the sterol is cholesterol or an analog thereof (e.g., thiocholesterol, epicholesterol, β-sitosterol (Si-Lip), stigmasterol (St-Lip), or lanosterol (La-Lip)). The concentration of the sterol in the compositions described herein may be, e.g., from about 1% to about 50% (e.g., from about 5% to about 45%, from about 10% to about 40%) of total lipid composition.

The sterol and phospholipid may be present in the particles in a weight ratio of, e.g., from about 0.01 to about 0.5 sterol:phospholipid. For example, the weight ratio of sterol:phospholipid may be, e.g., from about 0.01 to about 0.1, from about 0.1 to about 0.2, from about 0.2 to about 0.3, from about 0.3 to about 0.4, from about 0.4 to about 0.5, from about 0.01 to about 0.2, from about 0.01 to about 0.3, from about 0.01 to about 0.4, from about 0.1 to about 0.15, from about 0.1 to about 0.25. For example, the weight ratio of sterol:phospholipid may be about 0.01, 0.02, 0.03, 0.04, 0.05, 0.06, 0.07, 0.08, 0.09, 0.1, 0.15, 0.2, 0.25, 0.3, 0.35, 0.4, 0.45, or 0.5.

Sterols also encompasses esterified derivatives thereof, sometimes referred to as sterol esters or stanol esters. Sterol esters are sterols esterified with a fatty acid, such as a long chain (e.g., C₆-C₂₄, e.g., C₁₀-C₂₄, e.g., C₁₄-C₂₄) fatty acid, such as octanoic acid, decanoic acid, undecanoic acid, lauric acid, myristic acid, palmitic acid, stearic acid, oleic acid, linoleic acid, and linolenic acid. Sterols and their esters may be fully saturated (e.g., hydrogenated). Pharmaceutical compositions containing sterols or their esters may include one or more of the foregoing components or a mixture thereof.

### Kits

The invention described herein provides kits for treating diseases using compositions containing a cannabinoid. A kit for treating an ophthalmic condition is provided. In the kit for treating an ophthalmic condition a first ophthalmic formulation containing a cannabinoid is provided along with a second ophthalmic formulation containing a cannabinoid. The first ophthalmic formulation containing a cannabinoid is administered during daytime and the second ophthalmic formulation containing a cannabinoid is administered prior to sleep. The second ophthalmic formulation containing a cannabinoid has a higher viscosity that the first ophthalmic formulation containing a cannabinoid. The first ophthalmic formulation containing a cannabinoid is an eye drop and the concentration of the cannabinoid in both ophthalmic formulations is between 0.01% to about 10% (e.g., from about 0.05% to about 9.5%, from about 0.1% to about 9%, from about 0.2% to about 8.5%, from about 0.4% to about 8%, from about 0.5% to about 7.5%, from about 1% to about 7%, from about 1.5% to about 6.5%, from about 2% to about 6%, from about 2.5% to about 5.5%, from about 3% to about 5%, from about 3.5% to about 4.5%, from about 4% to about 4.49%, e.g., about 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0%) by weight of the composition.

A kit for treating bacterial vaginosis or candidiasis is provided. The kit may include a pessary that is coated or that contains a pharmaceutical composition comprising a cannabinoid and a topical formulation that includes a cannabinoid.

The kit may further include instructions for use thereof.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Treatment of a Human Subject Suffering from an Ophthalmic Condition

A pharmaceutical composition containing a cannabinoid or a derivative thereof in the form of eye drops were applied to a human subject suffering from an ophthalmic condition for multiple days. Two randomized, parallel-group studies were conducted. Group 1 included 10 human subjects that received 1 drop of the pharmaceutical composition of Table 1, applied to the eye once per day, twice per day, three times per day, four times per day, five times per day, six times per day, seven times per day, eight times per day, nine times per day, ten times per day, eleven times per day, twelve times per day, or hourly for 1 week. Group 2 included 10 human subjects that received a placebo without the cannabinoid, or its derivatives applied to the eye once per day, twice per day, three times per day, four times per day, five times per day, six times per day, seven times per day, eight times per day, nine times per day, ten times per day, eleven times per day, twelve times per day, or hourly for 1 week. For each of the groups, the infection of the eye, redness, and/or inflammation, dryness, allergy, vision, size of epithelial defects, amount of epitheliopathy of the eye were measured. A decrease in infection of the eye, redness, and/or inflammation of the eye in Group 1 individuals as compared to Group 2 individuals was observed and was indicative of a positive response to the pharmaceutical composition for the ophthalmic condition.

TABLE 1
Eye drop formulation in the form of an emulsion

| | |
|---|---|
| CBD or derivative | 1% (w/w) |
| Cyclodextrin | 0.5% (w/w) |
| Glycerin | 0.2% (w/w) |
| Sterile phosphate buffered saline | q.s. |

### Example 2. Pharmaceutical Compositions Comprising Cannabinoid or its Derivatives for the Treatment and Prevention Dry Eye, Blepharitis, or Meibomianitis

TABLE 2
Eye drop formulation in the form of a solution

| | |
|---|---|
| CBD or derivative | 1% (w/w) |
| Cyclodextrin | 0.5% (w/w) |
| Glycerin | 0.2% (w/w) |
| Sterile phosphate buffered saline | q.s. |

TABLE 3
Eye drop formulation in the form of a suspension

| | | |
|---|---|---|
| CBD or derivative | 1% | (w/w) |
| Sodium acetate | 1% | (w/w) |
| Sodium chloride | 0.8% | (w/w) |
| Benzalkonium chloride | 0.005% | (w/w) |
| Hydroxypropyl methylcellulose | 2% | (w/w) |
| Polyoxyethylene hydrogenated castor oil | 0.5% | (w/w) |

| | |
|---|---|
| Hydrochloric Acid | suitable amount to pH 5.0 |
| Sterile purified water | q.s. |

TABLE 4
Eye drop formulation in the form of an emulsion

| | | |
|---|---|---|
| CBD or derivative | 1% | (w/v) |
| Poloxamer | 2.5% | (w/v) |
| Polyethylene glycol | 0.5% | (w/v) |
| Sodium chloride | 0.75% | (w/v) |

| | |
|---|---|
| Hydrochloric acid or Sodium hydroxide | suitable amount to pH 6.5 |
| Sterile purified water | q.s. |

TABLE 5
Eye drop formulation in the form of an in-situ gel

| | |
|---|---|
| CBD or derivative | 1% (w/w) |
| Poloxamer | 5% (w/w) |
| Polyethylene glycol | 4% (w/w) |
| Sodium chloride | 2.6% (w/w) |
| Hydrochloric acid | suitable amount to pH 6.5 |
| Sterile purified water | q.s. |

TABLE 6
Eye drop formulation in the form of an in-situ gel

| | |
|---|---|
| CBD or derivative | 1% (w/w) |
| Castor oil | 5% (w/w) |
| Polysorbate 80 | 4% (w/w) |
| Concentrated glycerol | 2.6% (w/w) |
| Hydrochloric acid | suitable amount to pH 6.5 |
| Sterile purified water | q.s. |

TABLE 7
Eye drop formulation in the form of a gel

| | | |
|---|---|---|
| CBD or derivative | 1.5% | (w/w) |
| Carbopol | 3.5% | (w/w) |
| Glycerin | 8% | (w/w) |
| EDTA | 0.5% | (w/w) |
| Benzalkonium chloride | 0.05% | (w/w) |
| Tyloxapol | 0.05% | (w/w) |
| Boric acid | 5% | (w/w) |
| Sodium chloride | 0.5% | (w/w) |

| | |
|---|---|
| Sterile purified water | q.s. |

TABLE 8
Eye drop formulation in the form of an ointment

| | | |
|---|---|---|
| CBD or derivative | 1% | (w/w) |
| White Petrolatum, U.S.P. | 50% | (w/w) |
| Propylene Glycol | 5% | (w/w) |
| Glycerin | 5% | (w/w) |
| Tween 20 | 2% | (w/w) |
| Benzalkonium chloride | 0.1% | (w/w) |

| | |
|---|---|
| Mineral oil | q.s. |

### Example 3. Treatment of a Human Subject Suffering from an Ophthalmic Condition

Pharmaceutical compositions containing a cannabinoid, such as CBD, or a derivative thereof, such as those described in Tables 1-8, can be applied to the eye of a human subject suffering from an ophthalmic condition, such as dry eye, blepharitis, or meibomianitis, for multiple days. Two randomized, parallel-group studies were conducted. Group 1 included 10 human subjects that received 1 dose of a liquid ophthalmic pharmaceutical composition containing a cannabinoid (e.g., CBD) applied to the eye hourly, b.i.d., t.i.d., q.i.d., or daily (q.d.) for 1 week. Group 2 received a placebo without CBD applied to the eye hourly, b.i.d., t.i.d., q.i.d., or daily (q.d.) for 1 week. Group 3 received 1 dose of a liquid ophthalmic pharmaceutical composition containing a cannabinoid, such as CBD, the composition applied to the eye hourly, b.i.d., t.i.d., q.i.d., or daily (q.d.) for 1 week, and 1 dose of a solid ophthalmic pharmaceutical composition administered at around bedtime. For each of the groups, the infection of the eye, redness, and/or inflammation, dryness, allergy, vision, size of epithelial defects, amount of epitheliopathy of the eye was measured and testimony from dry eye questionnaires was obtained. A decrease in infection of the eye, redness, and/or inflammation of the eye in Group 1 individuals as compared to Group 2 individuals was observed and was indicative of a positive response to the pharmaceutical composition for the ophthalmic condition.

### Example 4. Exemplary Micelle Formulation Including Cannabidiol, Analogs, Derivatives, or a Combination Thereof for the Treatment and Prevention of a Dermatological Condition

A micelle formulation of 15 g of CBD was dissolved in 95% ethanol (final volume of 30 ml). This ethanolic extraction solution was combined with an ethanolic solution of Lecithin-50 (30 ml), which was prepared by dissolving 15 grams of Lecithin-50 using small portions of 95% EtOH and bringing the final volume of the lipid/EtOH solution to 30 mL using 95% EtOH. The ethanolic solution of lipid and CBD was cooled to 10° C. and injected at a pressure of 50 psi (10 mL/min) into 540 mL of distilled water (25° C.) using a 100 mL Luer Lock syringe equipped with a 22-gauge needle. A pressure of 50 psi, and a flow rate of 10 mL/min was maintained during the injection process. The micellar suspension can be concentrated to 200 mL by rotary evaporation at 30 mm Hg keeping the temperature of the micellar suspension below 55° C. The final maximum CBD concentration was 50 g/L. Micelles of 200-400 nm in diameter were obtained and showed an aqueous core visible under oil immersion microscopy. The micelle size was determined by gel filtration, and by oil immersion light microscopy.

### Example 5. Treatment of a Human Subject Suffering from a Dermatological Condition with a Topical Composition Including CBD

A topical composition including CBD in the form of a shampoo, body wash, lotion, cream, or ointment was topically applied to the skin of a human subject suffering from a dermatological condition for multiple days. Two randomized, parallel-group studies were conducted. Group 1 included 10 human subjects that received the topical composition of Example 4, topically applied to the skin once a day (o.d.) or twice a day (b.i.d) for 1-12 weeks. Group 2 received a placebo without the CBD, or its derivatives topically applied to the skin once a day (o.d.) or twice a day (b.i.d) for 1-12 weeks. For each of the groups, the infection of the skin, redness, inflammation, size of lesion of the skin, texture of skin, and/or photographic evidence was compared for evaluation purposes. A decrease in infection of the skin, redness, and/or inflammation of the skin in Group 1 individual as compared to Group 2 individuals was observed and was indicative of a positive response to the topical composition for the dermatological condition.

### Example 6. Treatment and Prevention of Bacterial Vaginosis Using Pharmaceutical Compositions Including a Cannabinoid

Pharmaceutical compositions containing a cannabinoid, such as CBD, or a derivative thereof, such as those described in Table 9, can be applied to the vaginal cavity of a human subject suffering from bacterial vaginosis. Two randomized, parallel-group studies were conducted. Group 1 included 10 human subjects that were administered 1 dose of a vaginal composition containing CBD applied to the vulvovaginal area b.i.d. or daily (q.d.) for 1 week. Group 2 was administered a placebo without CBD applied to the vulvovaginal area b.i.d. or daily (q.d.) for 1 week. For each of the groups, a clinically acceptable response was measured as described above, as well as questionnaires filled out by the subjects. A clinically acceptable response in Group 1 individuals as compared to Group 2 individuals was observed and was indicative of a positive response to the pharmaceutical composition for treatment of bacterial vaginosis.

A patient suffering from bacterial vaginosis was administered a dose of at least one vaginal formulation containing a cannabinoid, such as CBD, or a derivative thereof. The vaginal formulation containing the cannabinoid, such as CDB, or derivative was a solid formulation, such as a capsule or suppository (Table 9). The vaginal formulation was administered to contact a mucosal surface in the vaginal cavity (e.g., vulvovaginal surface) using an applicator or by hand. With the patient in the supine position, the tip of the applicator was gently inserted high in the vagina, e.g., in the posterior vaginal fornix, and the solid formulation was released from the applicator by pushing on a plunger of the applicator or otherwise releasing the suppository or capsule. Alternatively, the capsule or suppository was inserted by the patient by hand without an applicator. The suppository or capsule melted upon contact with the mucosal surfaces of the vagina. The vaginal formulation was administered once, hourly, b.i.d., t.i.d., q.i.d., or daily (q.d.) for 1 day, alternatively 2 days, alternatively 3 days, alternatively 4 days, alternatively 5 days, alternatively 6 days, alternatively 1 week. The vaginal formulation may also be administered prior to sleep. The vaginal formulation was administered until a clinically acceptable response was obtained.

A patient suffering from bacterial vaginosis was administered a dose of at least one vaginal formulation containing a cannabinoid, such as CBD, or a derivative thereof. The vaginal formulation containing the cannabinoid, such as CDB, or derivative was of solid formulation, such as a cream, gel, or ointment (Table 9). The vaginal formulation was administered to contact a mucosal surface in the vaginal cavity (e.g., vulvovaginal surface) using an applicator that is optionally prefilled with a single unit dosage amount. With the patient in the supine position, the tip of the applicator was gently inserted high in the vagina, e.g., in the posterior vaginal fornix, and the solid formulation was released through the tip of the applicator by pushing on a plunger of the applicator. The vaginal formulation was administered once, hourly, b.i.d., t.i.d., q.i.d., or daily (q.d.) for 1 day, alternatively 2 days, alternatively 3 days, alternatively 4 days, alternatively 5 days, alternatively 6 days, alternatively 1 week. The vaginal formulation was also optionally administered at night before sleep. The vaginal formulation was administered until a clinically acceptable response was obtained.

TABLE 9
Exemplary pharmaceutical compositions including
a cannabinoid or its derivatives for the treatment
and prevention of bacterial vaginosis.
Vaginal cream formulation I

| | | |
|---|---|---|
| Cannabinoid or derivative | 1% | (w/w) |
| Stearyl alcohol | 5.5% | (w/w) |
| Cetyl alcohol | 3% | (w/w) |
| Polysorbate 60 | 3% | (w/w) |
| Isopropyl Myristate | 1% | (w/w) |
| Propylene Glycol | 20% | (w/w) |
| Benzoic Acid | 0.2% | (w/w) |
| Potassium Hydroxide | 0.055% | (w/w) |

| | |
|---|---|
| Sterile Phosphate Buffered saline | q.s. |

Vaginal cream formulation II

| | | |
|---|---|---|
| Cannabinoid or derivative | 5% | (w/w) |
| Sorbitol solution | 37.2% | (w/w) |
| EDTA | 0.05% | (w/w) |
| Mineral oil | 10% | (w/w) |
| PEG-30 dipolyhydroxystearate | 4% | (w/w) |
| Glyceryl monoisostearate | 2% | (w/w) |
| Microcrystalline wax | 0.40% | (w/w) |
| Methylparaben | 0.018% | (w/w) |
| Propylparaben | 0.05% | (w/w) |

| | |
|---|---|
| Sterile water | q.s. |

Suppository I

| | | |
|---|---|---|
| Cannabinoid or derivative | 1% | (w/w) |
| Xanthan Gum | 1% | (w/w) |
| Sodium Carboxymethylcellulose 7HF | 8% | (w/w) |
| Colloidal Silicon Dioxide | 1% | (w/w) |
| WECOBEE ® M | 12% | (w/w) |
| WECOBEE ® FS | 54% | (w/w) |

Suppository II

| | | |
|---|---|---|
| Cannabinoid or derivative | 4% | (w/w) |
| Glycerin | 1% | (w/w) |
| Sodium Carboxymethylcellulose | 0.5% | (w/w) |
| Glyceryl esters of mixtures of | 11% | (w/w) |
| saturated vegetable fatty acids | | |
| Microcrystalline wax | 7% | (w/w) |
| Sorbitan tristearate | 3% | (w/w) |

| | |
|---|---|
| Purified Water | q.s. |

Capsule Fill

| | | |
|---|---|---|
| Cannabinoid or derivative | 21% | (w/w) |
| Polyethylene Glycol 400 | 60.8% | (w/w) |
| Propylene Glycol | 4.5% | (w/w) |
| Polyethylene Glycol 4600 | 3.6% | (w/w) |

| | |
|---|---|
| Sterile purified water | q.s. |

Gel

| | | |
|---|---|---|
| Cannabinoid or derivative | 1% | (w/v) |
| Benzalkonium chloride | 0.15% | (w/v) |
| Polyoxyethyen-20-monocetyl ether (Brij 58) | 0.5% | (w/v) |
| Hydroxyethylcellulose (NATROSOL ™ 250 HX) | 2.5% | (w/v) |
| Diethylene glycol monoethyl ester | 10.0% | (w/w) |
| (TRANSCUTOL ® P) | | |

| | |
|---|---|
| Sterile purified water | q.s. |

### Example 7. Formulations for Skin Lightening Using Topical Composition Containing a Cannabinoid

### I. Micelle Formulation

15 g of a CBD or its analogs and derivatives and 15 g of hydroquinone were dissolved in 95% ethanol (final volume of 30 ml). This ethanolic extraction solution was combined with an ethanolic solution of Lecithin-50 (30 ml), which was prepared by dissolving 15 grams of Lecithin-50 using small portions of 95% EtOH and bringing the final volume of the lipid/EtOH solution to 30 mL using 95% EtOH. The ethanolic solution of lipid and CBD was cooled to 10° C. and was injected at a pressure of 50 psi (10 mL/min) into 540 mL of distilled water (25° C.) using a 100 mL Luer Lock syringe equipped with a 22 gauge needle. A pressure of 50 psi and a flow rate of 10 mL/min were maintained during the injection process. The liposomal suspension was then concentrated to 200 mL by rotary evaporation at 30 mmHg keeping the temperature of the liposomal suspension below 55° C. The final maximum CBD concentration was 50 g/L. Liposomes of 200-400 nm diameter were obtained and had an aqueous core visible under oil immersion microscopy. The liposomes size was determined by gel filtration and by oil immersion light microscopy.

### II. Ointment Formulation

10 mg of hydroquinone, 10 mg of a cannabinoid, 250 mg of PEG-4000, 650 mg of PEG-400, 10 mg of white Vaseline, 1.44 mg of methyl p-hydroxybenzoate, 0.18 mg of propyl p-hydroxybenzoate, and the balanced amount of purified water were mixed. The mixture was then be used to prepare an ointment formulation according to a conventional ointment preparation method.

### III. Lotion Formulation

Three parts by weight of propylene glycol, 0.1 parts by weight of carboxypolymer, a trace of a preservative, and the balanced amount of purified water were mixed by stirring while heating to a temperature of 80 to 85° C. The mixture was then loaded into a preparation unit, and then, an emulsifying machine was driven, and 1.0 part by weight of polysolvate 60, 0.5 parts by weight of sorbitan sesquiolate, 10.0 parts by weight of liquid paraffin, 1.0 part by weight of sorbitan stearate, 0.5 parts by weight of lipophilic glyceryl monostearate, 1.5 parts by weight of stearic acid, 1.0 part by weight of glyceryl stearate/PEG-400 stearate, and 0.2 parts by weight of triethanolamine were then heated to a temperature of 80° C. to 85° C., and then, loaded thereinto to perform emulsification. When the emulsifying was completely performed, the mixture was stirred by using an agitator while heat-cooling to a temperature of 50° C., and then, a trace of flavoring agent was added thereto. After cooling to a temperature of 45° C., a trace of pigment can be added thereto, and 4.0 parts by weight of hydroquinone and 4.0 parts by weight of a cannabinoid can be added thereto at a temperature of 35° C. and the resultant mixture was cooled to a temperature of 25° C. and aged.

### IV. Cream Formulation

0.3 parts by weight of carboxypolymer, 5.0 parts by weight of butylene glycol, 3.0 parts by weight of glycerin, and the balanced amount of purified water were mixed by stirring while heating to a temperature of 80° C. to 85° C., and the mixture was loaded into a preparation unit, and then, an emulsifying machine was driven. Then, 2.0 parts by weight of a stearic acid, 2.0 parts by weight of cetyl alcohol, 2.0 parts by weight of glyceryl monostearate, 0.5 parts by weight of polyoxyethylene sorbitan monostearate, 0.5 parts by weight of sorbitan sesquiolate, 1.0 part by weight of wax, 1.0 part by weight of glyceryl monostearate/glyceryl stearate/polyoxyethylene stearate, 4.0 parts by weight of liquid paraffin, 4.0 parts by weight of squalane, and 4.0 parts by weight of caprylic/capric triglyceride were heated to a temperature of 80° C. to 85° C. and were then loaded thereinto. Then, 0.5 parts by weight of triethanolamine was loaded thereinto and emulsifying was performed thereon. When the emulsifying was completely performed, the resultant mixture was stirred by using an agitator while cooling to a temperature of 35° C., and then, 4.0 parts by weight of hydroquinone and 4.0 parts by weight of a cannabinoid were loaded thereinto and cooled to a temperature of 25° C. and aged.

### V. Foam Formulation

30.0 parts by weight of TEA-cocoyl glutamate, 10.0 parts by weight of disodium laureth sulfosuccinate glycerin, 10.0 parts by weight of glycerin, 2.0 parts by weight of cocamide DEA, 1.0 part by weight of PEG-120 methylglucose dioliate, 0.5 parts by weight of methyl gluceth-20, 0.5 parts by weight of PEG-150 pentaerythrityl tetra stearate, 0.05 parts by weight of tetrasodium EDTA, and a trace of preservative were sequentially added into a preparation unit, and were heated to a temperature of 60° C. to 65° C. and were stirred for 15 minutes. When the stirring was completely performed, some purified water was added therein, and the resultant mixture was stirred for 30 minutes. Then, some more purified water was slowly added thereinto, and the resultant mixture was stirred for 30 minutes, and cooled to a temperature of 35° C. 4.0 parts by weight of hydroquinone, 4.0 parts by weight of a cannabinoid, and a flavoring agent was then added thereunto, and the resultant mixture was cooled to a temperature of 25° C. and aged.

### Example 8. Treatment of Skin with a Topical Composition Containing a Cannabinoid to Lighten the Skin

A topical composition, such as any one of Example 7, in the form of a shampoo, body wash, lotion, cream, or ointment was topically applied to the skin of a human subject for multiple days to lighten or whiten the skin of the human subject. The topical compositions including at least a skin lightening agent and a cannabinoid. Two randomized, parallel-group studies were conducted. Group 1 included 10 human subjects that received the topical composition, topically applied to the skin once a day (o.d.) or twice a day (b.i.d) for 1-12 weeks. Group 2 received a placebo without the cannabinoid, or its derivatives topically applied to the skin once a day (o.d.) or twice a day (b.i.d) for 1-12 weeks. For each of the groups, the color of the skin, redness, inflammation, size of lesion of the skin, texture of skin, any infection, and/or photographic evidence were compared for evaluation purposes. A decrease in color of the skin, redness, and/or inflammation of the skin in Group 1 individual as compared to Group 2 individuals was observed and was indicative of a positive response to the topical composition for the dermatological condition.

### Example 9. Treatment of a Human Subject Suffering from a Dermatological Condition Caused by Overexposure to Ultraviolet Radiation

A patient suffering from a dermatological condition caused by overexposure to ultraviolet radiation may apply a topical composition in the form of a shampoo, body wash, lotion, cream, or ointment to the affected area of their skin. The topical composition was be applied once a day (o.d.), twice a day (b.i.d.), three times a day (t.i.d.), four times a day (q.i.d.), or daily for 1 day, alternatively 2 days, alternatively 3 days, alternatively 4 days, alternatively 5 days, alternatively 6 days, alternatively 1 week, alternatively 2 weeks, alternatively up to 12 weeks. The patient may optionally be applying the topical composition to lighten skin color, preventing and/or reducing excessive darkening, prevent liver spots, preventing and/or reducing freckles, preventing and/or reducing hyperpigmentation, preventing and/or reducing age spots, preventing and/or reducing sunspots, or preventing and/or reducing seborrheic keratosis.

### Example 10. Formulations and Treatment and/or Prevention of Acne Using a Topical Composition Containing a Cannabinoid

An aqueous phase was prepared by adding together and stirring purified water (in sufficient quantity), glycerin, methyl paraben, gluconolactone, and edetate disodium to create a clear dispersion. Carbopol 971P was added under homogenization. Sodium lauryl sulfate (at a concentration of around 90%) was added to this dispersion under homogenization, maintaining the temperature at about 70±5° C. The oil phase was prepared by mixing stearyl alcohol, cetyl alcohol, caprylic/capric triglyceride, and tocofersolan. These ingredients were mixed and melted. Propyl paraben and butylated hydroxyl toluene were added to the dispersion, maintaining the temperature at about 70±5° C. throughout the process. The oil phase was then mixed slowly with the aqueous phase under homogenization to prepare the emulsion and cooled down to about 30±2° C. The pH of the emulsion was adjusted to 5.2±0.2 by adding triethanolamine under homogenization. The drug dispersion was prepared by adding tazarotene, cannabinoid, and the remaining sodium lauryl sulfate to a sufficient quantity of purified water with stirring. The drug dispersion was then mixed with the emulsion under homogenization. The pH of the product was adjusted to 6.3±0.2 by adding triethanolamine and purified water was added to make up the final weight.

TABLE 10
Formulations comprising a cannabinoid for
the treatment and/or prevention of acne.

| Ingredients | % w/w |
|---|---|
| Tazarotene | 0.1 |
| Cannabinoid or derivative | 1.0 |
| Sodium lauryl sulfate | 2.0 |
| Steraryl alcohol | 2.0 |
| Cetyl alcohol | 1.2 |
| Gluconolactone | 0.25 |
| Glycerin | 4.0 |
| Tocofersolan | 0.5 |
| Carbopol 971P | 0.25 |
| Propyl paraben | 0.03 |
| Methyl paraben | 0.20 |
| Edetate disodium | 0.1 |
| Butylated Hydroxy Toluene | 0.05 |
| Caprylic/Capric Triglyceride | 3.5 |
| Triethanolamine | q.s. to pH 6.3 |
| Purified Water | q.s. to 100 |

A topical composition, such as one exemplified in Table 10, in the form of a wash, lotion, cream, or ointment was topically applied to the skin of a human subject for multiple days to treat acne. The topical compositions including at least an acne agent and a cannabinoid. Two randomized, parallel-group studies were conducted. Group 1 included 10 human subjects received the topical composition, topically applied to the skin once a day (o.d.) or twice a day (b.i.d) for 1-12 weeks. Group 2 received a placebo without the cannabinoid, or its derivatives topically applied to the skin once a day (o.d.) or twice a day (b.i.d) for 1-12 weeks. For each of the groups, the appearance of acne, infection of the skin, redness, inflammation, size of lesion of the skin, texture of skin, and/or photographic evidence were compared for evaluation purposes. A decrease in the appearance of acne, infection of the skin, redness, and/or inflammation of the skin in Group 1 individual as compared to Group 2 individuals was observed and was indicative of a positive response to the topical composition for the dermatological condition.

A patient suffering from acne may apply a topical composition in the form of a wash, lotion, cream, or ointment to the affected area of their skin. The topical composition was applied once a day (o.d.), twice a day (b.i.d.), three times a day (t.i.d.), four times a day (q.i.d.), or daily for 1 day, alternatively 2 days, alternatively 3 days, alternatively 4 days, alternatively 5 days, alternatively 6 days, alternatively 1 week, alternatively 2 weeks, alternatively up to 12 weeks. The patient may apply the topical composition to treat and/or prevent acne, and/or the appearance of whiteheads, blackheads (comedones), pimples, papules, pustules, cysts, nodules, greasy skin.

### Example 11. Preparation of Phospholipid Preliminary Liposomes

Preparation and visual characterization of phospholipid liposomal compositions was performed. Three variant formulations, Fa1, Fa2, and Fa3 were prepared using 1,2-distearoyl-sn-glycero-3-phosphocholine (DSPC), cannabidiol (CBD) and optionally cholesterol. Fa1, was prepared by mixing DSPC (1 mg/ml), CBD (30 mg/ml), and cholesterol (0.37 mg/ml) in ethanol, followed by injection into DI water (Table 11). Fa2, was prepared by mixing DSPC (1 mg/ml), and CBD (30 mg/ml) in ethanol and injecting it into DI water. Fa3, was prepared by mixing DSPC (2 mg/ml), CBD (30 mg/ml), and cholesterol (0.73 mg/ml) in ethanol followed by injection into DI water.

TABLE 11
Preliminary liposomal formulations Fa.

| Formulation No. | Formulation Contents | Method |
|---|---|---|
| Fa1 | DSPC 1 mg/ml, CBD 30 mg/ml and | Ethanol |
| | Chol 0.37 mg/ml (15 molar % of DSPC) | Injection |
| Fa2 | DSPC 1 mg/ml, CBD 30 mg/ml | Ethanol |
| | | Injection |
| Fa3 | DSPC 2 mg/ml, CBD 30 mg/ml and | Ethanol |
| | Chol 0.73 mg/ml (15 molar % of DSPC) | Injection |

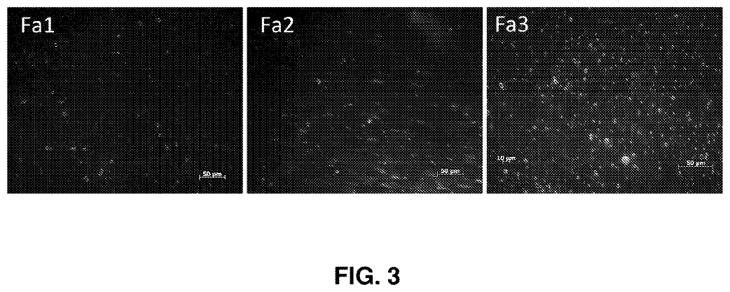

Visual characterization of the liposomal suspensions, Fa1, Fa2, and Fa3, yielded all suspensions with cloudy appearance and having some precipitates (Table 12). The relative abundance of liposomes was qualitatively characterized using light microscopy (FIG. 3) and it was observed that the process of formulating Fa3 generated the highest concentration of liposomes.

TABLE 12
Visual appearance of preliminary liposomal formulations Fa.

| Formulation No. | Observation |
|---|---|
| Fa1 | Cloudy Suspension with precipitate |
| Fa2 | Cloudy Suspension with precipitate |
| Fa3 | Cloudy Suspension with precipitate |

### Example 12. Effect of Temperature on Phospholipid Preliminary Liposomes

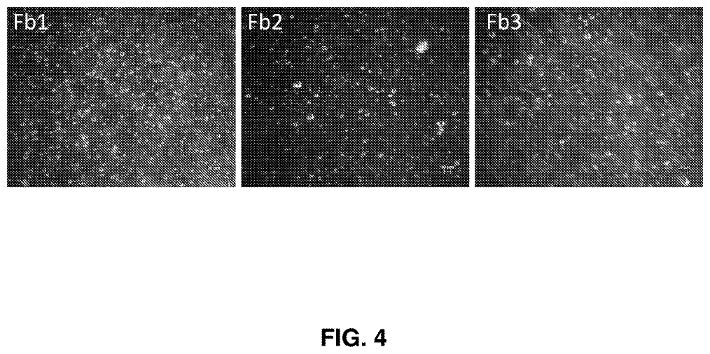

To overcome the precipitation observed in the Fa formulations experiments, liposomal suspensions were prepared with differing temperature conditions. The effect of temperature of an aqueous solvent on the dissolution and visual appearance of liposomal suspensions was assessed. Three formulations Fb1, Fb2, and Fb3 were prepared as in Table 3. Fb1 was prepared in deionized water at room temperature (20° C.) and ethanol at 4° C. The ethanol solution comprising of DSPC (2 mg/ml), CBD (30 mg/ml) and cholesterol (0.73 mg/ml, 15 molar % of DSPC) was introduced into the water using immersed injection technique. The resultant suspension had a cloudy appearance with precipitates of CBD and non-dispersed lipid. Fb2 was prepared using deionized water at 20° C. followed by an air injection of DSPC (2 mg/ml), CBD (30 mg/ml) and cholesterol (0.73 mg/ml, 15 molar % of DSPC). The resultant suspension also had a cloud appearance with precipitates of CBD and non-dispersed lipid. Fb3 was prepared using deionized water at 20° C. followed by an immersed injection of CBD and non-dispersed lipid. Fb3 had a cloudy appearance with observable precipitates of CBD and non-dispersed lipid (FIG. 4).

TABLE 13
Water temperature and method of ethanol
injection used for Fb formulations.

| Formulation No. | Method |
|---|---|
| Fb1 | Water (20° C.), ethanol (4° C.), immersed injection |
| Fb2 | Air injection, water (20° C.) |
| Fb3 | Immersed injection, water (20° C.) |

### Example 13. Effect of CBD Concentration and Method of Preparation

The preparation of lipid nanoparticle formulations (Fc1-Fc9, Table 14) was performed to examine the effect of cannabidiol (CBD) concentration, temperature, and homogenization method on the visual appearance, particle size, and zeta potential of the nanoparticles in suspensions.

TABLE 14
Formulations c (Fc) of CBD encapsulated in lipid nanoparticles prepared
at room temperature (20° C.) or at 60° C.

| | | Temperature and |
|---|---|---|
| Formulation No. | Formulation Content | Homogenization |
| Fc1 | DSPC (2 mg/ml), CBD (30 mg/ml), Cholesterol | Heated (60° C.) |
| | (0.73 mg/ml - 15 molar % of DSPC) | |
| Fc2 | DSPC (2 mg/ml), CBD (30 mg/ml), Cholesterol | Room Temp (20° C.) |
| | (0.73 mg/ml - 15 molar % of DSPC) | |
| Fc3 | DSPC (2 mg/ml), CBD (2 mg/ml), Cholesterol | Room Temp (20° C.) |
| | (0.73 mg/ml - 15 molar % of DSPC) | |
| Fc4 | DSPC (2 mg/ml), CBD (2 mg/ml), Cholesterol | Heated (60° C.) |
| | (0.73 mg/ml - 15 molar % of DSPC) | |
| Fc5 | DSPC (2 mg/ml), CBD (2 mg/ml), Cholesterol | Homogenized and Room |
| | (0.73 mg/ml - 15 molar % of DSPC) | Temp (20° C.) |
| Fc6 | DSPC (2 mg/ml), CBD (2 mg/ml), Cholesterol | Heated (60° C.) and |
| | (0.73 mg/ml - 15 molar % of DSPC) | Dropwise |
| Fc7 | CBD (2 mg/ml) (control) | Room Temp (20° C.) |
| Fc8 | DSPC (2 mg/ml), CBD (2 mg/ml), Cholesterol | Homogenized and |
| | (0.73 mg/ml - 15 molar % of DSPC) | Heated (60° C.) |
| Fc9 | DSPC (2 mg/ml), CBD (30 mg/ml), Cholesterol | Heated (60° C.) and |
| | (0.73 mg/ml - 15 molar % of DSPC) | Dropwise |

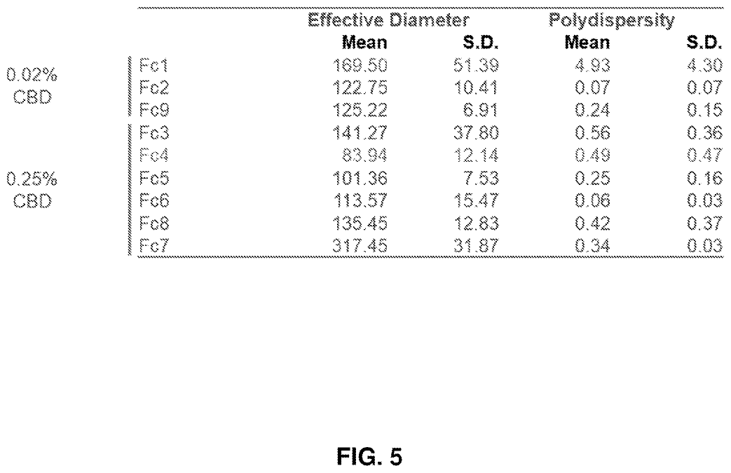

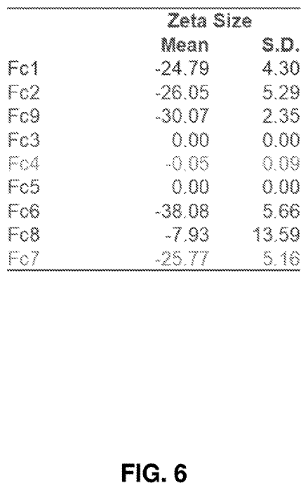

Two concentrations of CBD were used in the formulations, 2 mg/ml and 30 mg/ml, while the concentration of 1,2-distearoyl-sn-glycero-3-phosphocholine (DSPC) was constant for all formulations at 2 mg/ml. Cholesterol concentration was also maintained constant at 0.73 mg/ml (15 mol % of DSPC). Fc1 and Fc4 were formulations that directly compare CBD concentration while the preparation conditions where consistent e.g., temperature and concentrations of DSPC and cholesterol. The effect of the temperature was also assessed. Fc1-Fc4 examined the difference in suspension appearance, precipitates, particle size, and zeta potential at either room temperature (20° C.) or at elevated temperature (60° C.) (Table 15 and FIGS. 5-6). Homogenization of the suspension was performed in formulations Fc5 and Fc8 at either 20° C. (Fc5) or at 60° C. (Fc8). Visual inspection of suspension appearance, precipitates, particle size, and zeta potential at either room temperature (20° C.) or at elevated temperature (60° C.) (Table 15 and FIGS. 5-6). Formulations Fc6 and Fc9 examined the effect of CBD concentration, 2 mg/ml and 30 mg/ml, on the suspension appearance, precipitates, particle size, and nanoparticle zeta potential while the suspension was kept at 60° C. and CBD was added in dropwise manner (Table 15 and FIGS. 5-6). Formulation Fc7 was a control drug-only formulation.

TABLE 15
Visual appearance and the presence of precipitates of formulations Fc1-Fc9.

| Formulation No. | Visual Observations |
|---|---|
| Fc1 | Opaque/cloudy Suspension with precipitate |
| Fc2 | Opaque/cloudy Suspension with precipitate |
| Fc3 | Translucent/milky/less Cloudy Suspension with less precipitate |
| Fc4 | Translucent/milky/less Cloudy Suspension with less precipitate |
| Fc5 | Translucent/milky/less Cloudy Suspension with less precipitate |
| Fc6 | Translucent/milky/less Cloudy Suspension with less precipitate |
| Fc7 | Translucent/less Cloudy Suspension with crystal precipitate |
| Fc8 | Translucent/milky/less Cloudy Suspension with less precipitate |
| Fc9 | Cloudy Suspension with precipitate |

Particle size of formulations Fc1-Fc9 was assessed as mean effective diameter (nm) and mean polydispersity with standard deviation margins (FIG. 5). The smallest size of nanoparticles was observed from formulation Fc1. The method of preparation of formulations Fc1 and Fc4 only differed by the concentration of CBD used. The higher concentration of CBD resulted in larger particles likely due to unentrapped particulates of CBD which is consistent with the largest size of particles observed in formulation Fc7. The method of preparation of formulation Fc1 resulted also in a mixture of smaller lipid nanoparticles and larger unentrapped CBD particulates reflected in the large polydispersity seen in Fc1. Formulation Fc4 showed the optimal method to proceed with given the smallest nanoparticle size and low polydispersity value (FIG. 5).

Zeta potential was also measured for formulations Fc1-Fc9 (FIG. 6). The mean zeta potential in mV was recorded along with the standard deviation (S.D.) from triplicate measurements, where each measurement recorded is an average of 10 runs. The zeta potential value of formulation Fc4 was −0.05 mV with a S.D. of 0.09, consistent with the selection of Fc4 as the optimal formulation because it confirms that CBD was loaded into the nanoparticles and the surface charge of the nanoparticles is near neutral.

### Example 14. Effect of Poloxamer on Polymer Particle Composition

The effect of poloxamer concentration on the suspension clarity, amount of precipitate, particle size, suspension pH, and suspension viscosity were examined using PLURONIC® F127 as the poloxamer. Micellar formulations Fd1-Fd6 where prepared using 0.5 g/100 ml CBD and homogenized at the lowest speed of the homogenizer for 2 minutes. PLURONIC® F127 concentration was varied from 1% to 10% w/v from formulations Fd1 to Fd6 respectively as seen in Table 16.

TABLE 16
Formulations of PLURONIC ® F127 micelles with CBD.

| Formulation No. | Formulation Components | Method |
|---|---|---|
| Fd1 | 1% F127 + 0.5% CBD | Homogenisation for 2 minutes (speed: lowest) |
| Fd2 | 2% F127 + 0.5% CBD | Homogenisation for 2 minutes (speed: lowest) |
| Fd3 | 3% F127 + 0.5% CBD | Homogenisation for 2 minutes (speed: lowest) |
| Fd4 | 4% F127 + 0.5% CBD | Homogenisation for 2 minutes (speed: lowest) |
| Fd5 | 5% F127 + 0.5% CBD | Homogenisation for 2 minutes (speed: lowest) |
| Fd6 | 10% F127 + 0.5% CBD | Homogenisation for 2 minutes (speed: lowest) |

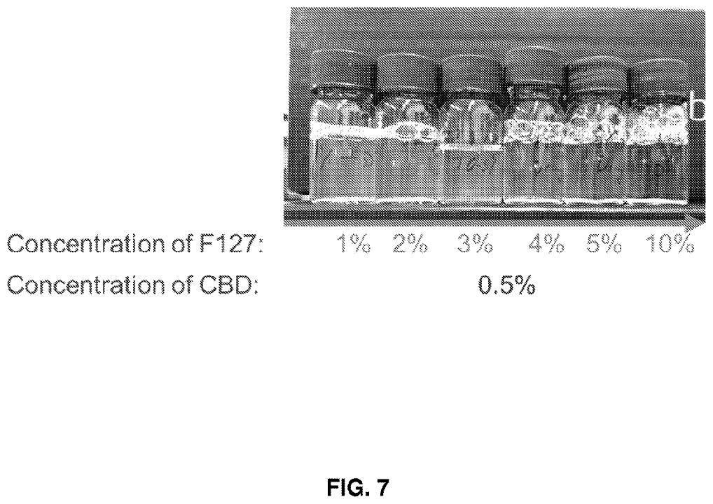

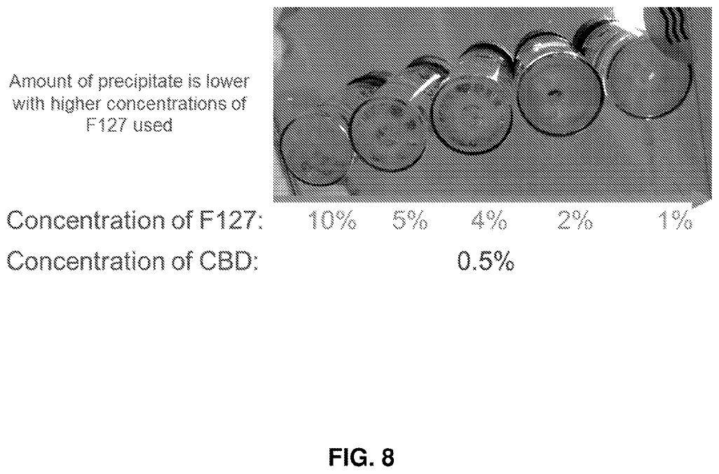

Formulations Fd1-Fd6 all had a translucent appearance (FIGS. 7-8), as opposed to the cloudy appearance observed in lipid-only nanoparticles. Varying amount of precipitate was observed that correlated with the concentration of PLURONIC® F127 used (Table 17).

TABLE 17
Observed micellar suspension transparency
and presence of precipitates.

| Formulation No. | Visual Appearance and Precipitate Observation |
|---|---|
| Fd1 | Translucent, milky suspension, precipitate |
| Fd2 | Translucent, less milky than Fd1 suspension, |
| | less precipitate than Fd1 |
| Fd3 | Translucent, less milky than Fd2 suspension, |
| | less precipitate than Fd2 |
| Fd4 | Translucent, less milky than Fd3 suspension, |
| | less precipitate than Fd3 |
| Fd5 | Translucent, less milky than Fd4 suspension, |
| | less precipitate than Fd4 |
| Fd6 | Transparent, minimal precipitate |

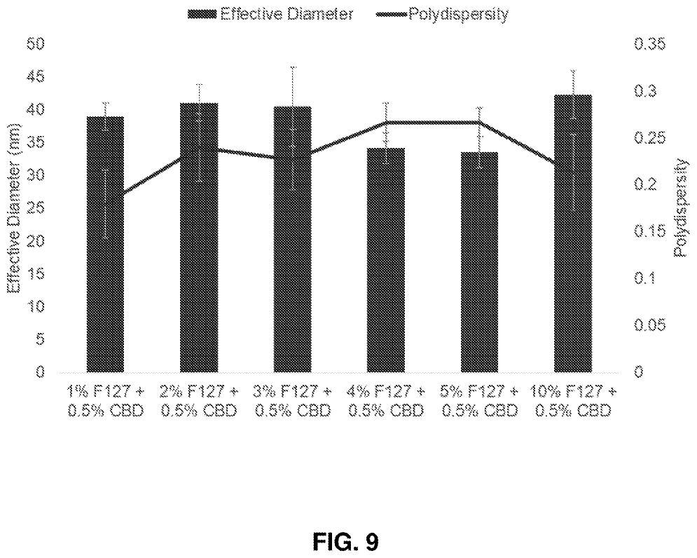

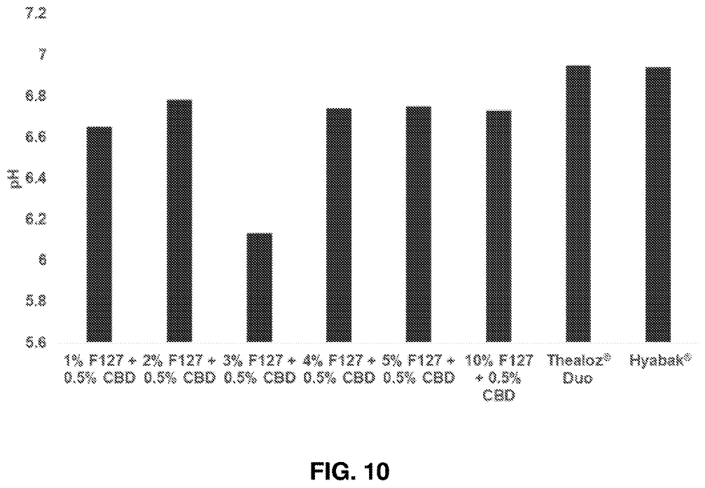

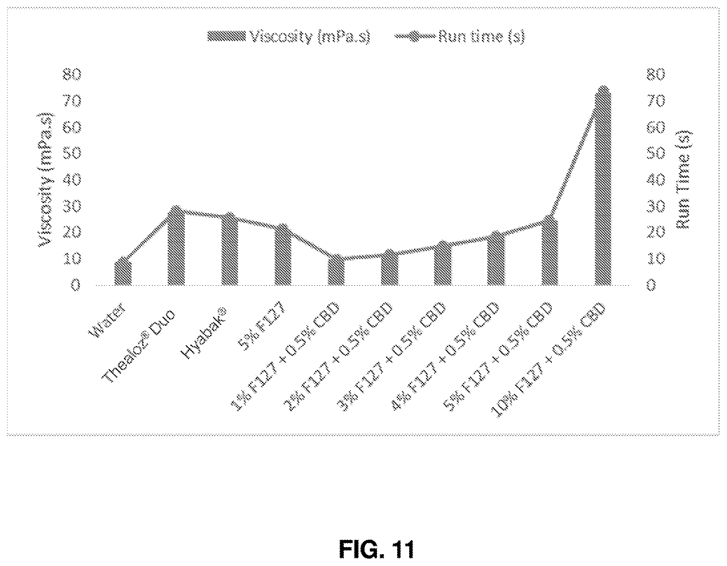

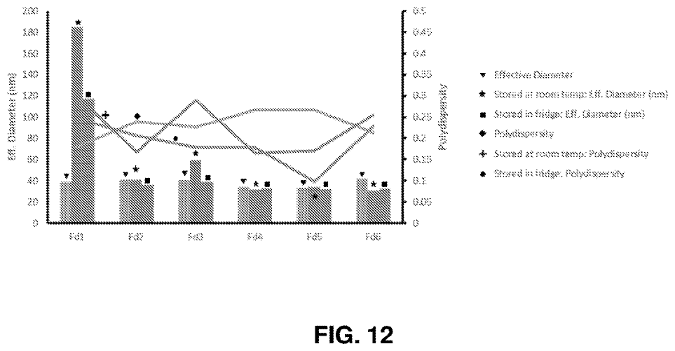

Suspensions with higher PLURONIC® F127 showed higher transparency and also less amount of precipitate indicating the effect of PLURONIC® F127 on the dissolution of CBD (FIGS. 7-8). Particle size was characterized, and all formulations resulted in micelles of similar size below 50 nm (FIG. 9). The pH and viscosity of formulations Fd1-Fd6 were measured and compared to two commercially available eye drop solutions, THEALOZ® Duo and HYABAK® (FIGS. 10-11). All PLURONIC® F127 formulations, except for Fd3, had pH values close to neutral pH. Fd3 showed lower pH closer to 6.2 (FIG. 10). Viscosity was also lower than the commercially available eyedrop solutions for all but one Fd formulations. Fd10 had a significantly higher viscosity that could be attributed to it being the formulation with the highest CBD concentration (FIG. 11). Stability of Fd1-Fd6 was also characterized on the day of preparation, and after 30 days of storage at either room temperature (20° C.) or at 4° C. (FIG. 12).

### Example 15. Formulation of Lipid-Polymer Composite Particles

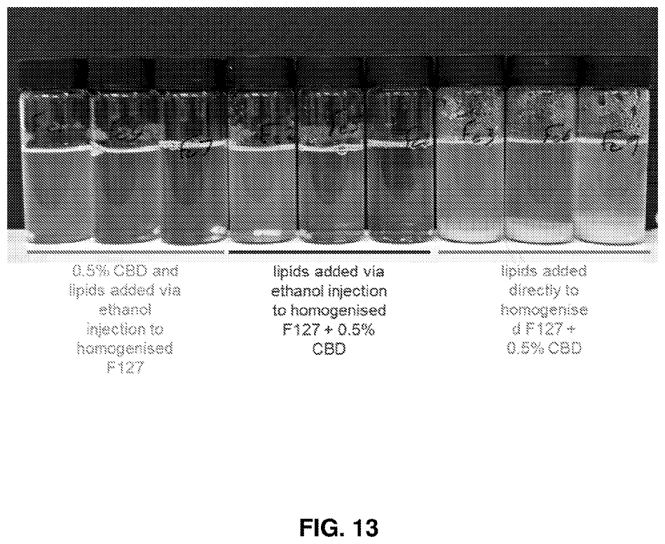

Lipid-polymer composite particles were prepared with PLURONIC® F127 concentrations 1%, 3% and 5% w/v, with 0.5% CBD concentration, with 2 mg/ml 1,2-distearoyl-sn-glycero-3-phosphocholine (DSPC) and 0.73 mg/ml cholesterol into formulations Fe1-Fe9 (Table 18). Characterization of the Fe formulations included visual inspection of transparency and presence of precipitates, particle size and polydispersity, suspension pH, suspension tonicity, size distribution, and size stability after 19 days of storage. Formulations Fe1, Fe4 and Fe7 where homogenized with all of the components at 60° C. with DSPC and cholesterol added to the suspension mixture via ethanol injection. Formulations Fe2, Fe5, and Fe8 were prepared in two steps. First, PLURONIC® F127 was homogenized with CBD followed by addition of DSPC and cholesterol via ethanol injection. Formulations Fe3, Fe6 and Fe9 were also prepared in two separate steps. First, PLURONIC® F127 was homogenized with CBD. Then a powder mixture of DSPC and cholesterol was added directly to the PLURONIC® F127-CBD suspension at 60° C. Upon visual inspection all suspensions were translucent (Table 19 and FIG. 13). Formulations Fe1, Fe4, and Fe7 had a milky appearance with less milky appearance with increased PLURONIC® F127 concentration. Formulations Fe2, and Fe5 also showed decreased precipitates and milky appearance with increased PLURONIC® F127 concentration. Fe8 appeared transparent and had no observable precipitates (Table 19 and FIG. 13). Formulations Fe3, Fe6, and Fe9 also had a milky appearance (FIG. 13) with a soft/cloudy precipitate observed in Fe6, and Fe9.

TABLE 18
Formulation of lipid-polymer composite particles.

| Formulation No. | Formulation Components and Method of Preparation |
|---|---|
| Fe1 | Homogenised 1% F127. 0.5% CBD and lipids (2 mg/ml DSPC and 0.73 mg/ml |
| | cholesterol) added via ethanol injection at 60° C. |
| Fe4 | Homogenised 3% F127. 0.5% CBD and lipids (2 mg/ml DSPC and 0.73 mg/ml |
| | cholesterol) added via ethanol injection at 60° C. |
| Fe7 | Homogenised 5% F127. 0.5% CBD and lipids (2 mg/ml DSPC and 0.73 mg/ml |
| | cholesterol) added via ethanol injection at 60° C. |
| Fe2 | Homogenised 1% F127 + 0.5% CBD. Lipids (2 mg/ml DSPC and 0.73 mg/ml |
| | cholesterol) added via ethanol injection at 60° C. |
| Fe5 | Homogenised 3% F127 + 0.5% CBD. Lipids (2 mg/ml DSPC and 0.73 mg/ml |
| | cholesterol) added via ethanol injection at 60° C. |
| Fe8 | Homogenised 5% F127 + 0.5% CBD. Lipids (2 mg/ml DSPC and 0.73 mg/ml |
| | cholesterol) added via ethanol injection at 60° C. |
| Fe3 | Homogenised 1% F127 + 0.5% CBD. Lipids (2 mg/ml DSPC and 0.73 mg/ml |
| | cholesterol) powder added directly at 60° C. |
| Fe6 | Homogenised 3% F127 + 0.5% CBD. Lipids (2 mg/ml DSPC and 0.73 mg/ml |
| | cholesterol) powder added directly at 60° C. |
| Fe9 | Homogenised 5% F127 + 0.5% CBD. Lipids (2 mg/ml DSPC and 0.73 mg/ml |
| | cholesterol) powder added directly at 60° C. |

TABLE 19
Observed Fe formulation transparency and presence of precipitates.

| Formulation No. | Formulation Components and Method of Preparation |
|---|---|
| Fe1 | Translucent, milky suspension with precipitate |
| | (less than Fe3, Fe6 and Fe9) |
| Fe4 | Translucent, less milky than Fe1 suspension with |
| | precipitate (less than Fe3, Fe6 and Fe9) |
| Fe7 | Translucent, less milky than Fe4 suspension with |
| | precipitate (less than Fe3, Fe6 and Fe9) |
| Fe2 | Translucent and milky with precipitate |
| Fe5 | Translucent and milky, no precipitate |
| Fe8 | Transparent, no precipitate |
| Fe3 | Translucent, milky suspension with precipitate |
| Fe6 | Translucent, less milky than Fe3 suspension with |
| | soft/cloudy precipitate |
| Fe9 | Translucent, less milky than Fe6 suspension with |
| | soft/cloudy precipitate |

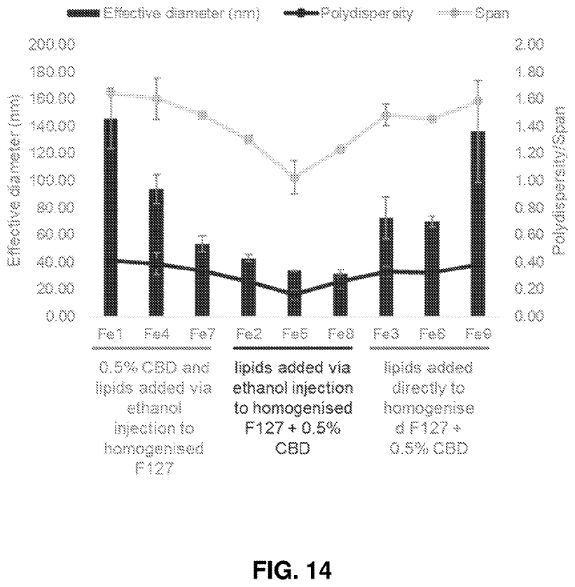

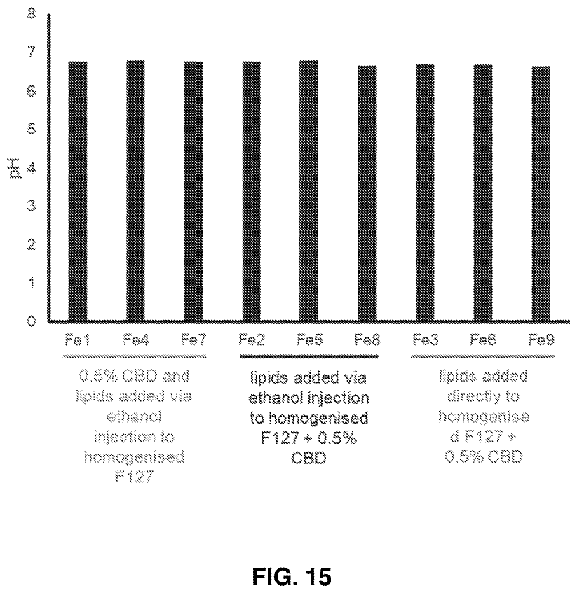

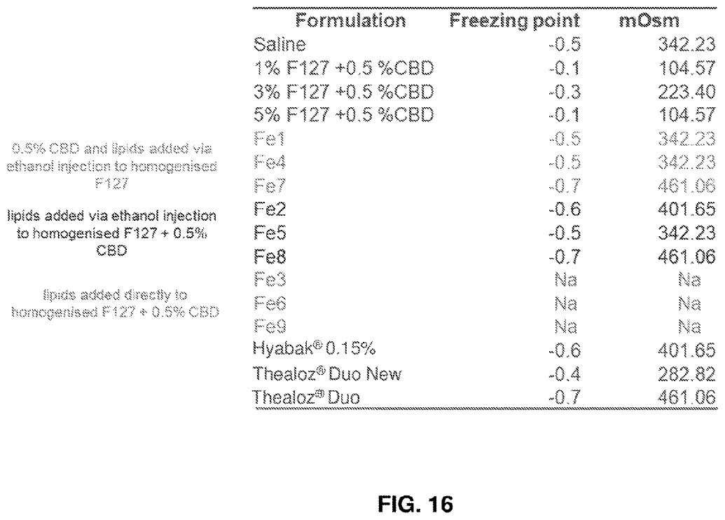

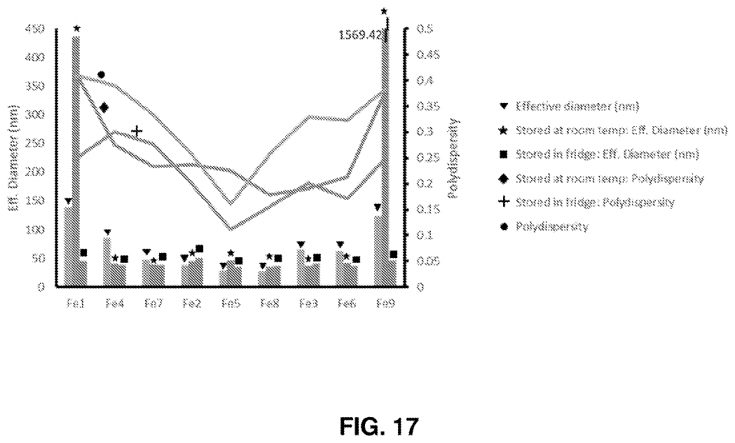

In formulations Eel, Fe4, and Fe7 the particle size and polydispersity decreased with increased PLURONIC® concentration. This trend was also observed in formulations Fe2, Fe5, and Fe8. In formulations Fe3, Fe6, and Fe9 the concentration of PLURONIC® did not correlate with the particle size. This was potentially due to the reduced dissolution of DSPC and cholesterol via the direct addition of these in powder form (FIG. 14). Particle span values for all formulations ranged between approximately 1.00 to 1.60 (FIG. 14) while polydispersity was lowest for particles in formulation Fe5. In general, formulations Fe2, Fe5, and Fe8 had the lowest mean particle size. pH across all formulations was close to neutral pH (FIG. 15). Tonicity of the formulations was also measured and compared to saline control solution, suspensions of PLURONIC® F127 and CBD only, and with commercially available eye drop solutions HYABAK® 0.15%, and THEALOZ® Duo (FIG. 16). Size distribution was characterized for all formulations showing two particle size populations. Formulations Fe5 and Fe8 showed a decreased abundance of the population of particles of larger size compared to the population of particles of lower size. Stability of Eel-Fe6 was also characterized on the day of preparation, and after 19 days of storage at either room temperature (20° C.) or at 4° C. (FIG. 17). All formulations showed stable size stability at 19 days in either room temperature (20° C.) or at 4° C. except for Fe1 and Fe9. Visual inspection of all formulation was performed at 19 days from preparation with minimal to moderate color change to an orange/pink hue for all the formulations store at room temperature. Formulations stored at 4° C. did not have observable color or appearance changes from the day of preparation (Table 20).

TABLE 20
Color change in formulations Fe.

| | Observation after 19 days from |
|---|---|
| Formulation No. | preparation stored at 20° C. |
| Fe1 | minimal color change to orange/pink |
| Fe4 | minimal color change to orange/pink |
| Fe7 | minimal color change to orange/pink |
| Fe2 | color change to orange/pink |
| Fe5 | color change to orange/pink |
| Fe8 | color change to orange/pink |
| Fe3 | color change to orange/pink |
| Fe6 | color change to orange/pink |
| Fe9 | color change to orange/pink |

### Example 16. Method Optimization of Chosen Formulations

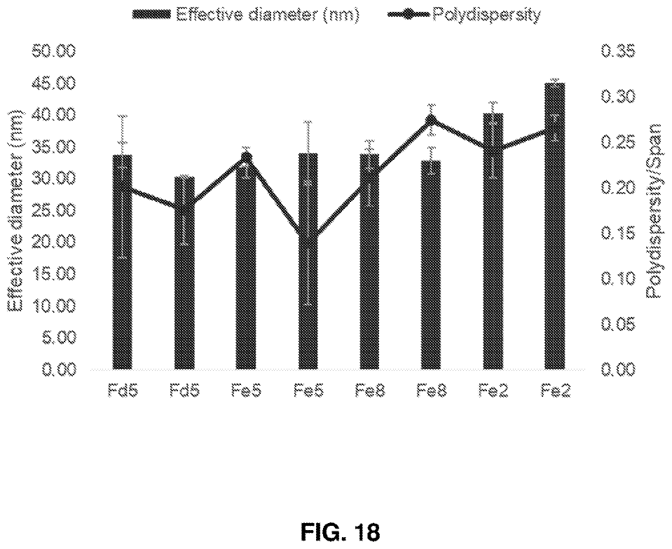

A selection of formulations was further optimized (Table 21). Duplicates of each formulation Fd5, Fe5, Fe8, and Fe2 were prepared, and the particle size and polydispersity were characterized (FIG. 18). Formulations Fd5, Fe5, and Fe8 yielded optimal particle size and polydispersity.

TABLE 21
Lipid-polymer composite particle optimization formulations.

| Formulation | | |
|---|---|---|
| No. | Formulation | Method |
| Fd5 | F127 5% + 0.5% | Homogenise for 2 min |
| | CBD | |
| Fd5 | F127 5% + 0.5% | Homogenise for 2 min |
| | CBD | |
| Fe5 | F127 3% + 0.5% | Homogenise for 2 min, ethanol injection |
| | CBD + Lipids | 7 sec/750 μl at 60° C., evaporate 2 h |
| Fe5 | F127 3% + 0.5% | Homogenise for 2 min, ethanol injection |
| | CBD + Lipids | 7 sec/750 μl at 60° C., evaporate 2 h |
| Fe8 | F127 5% + 0.5% | Homogenise for 2 min, ethanol injection |
| | CBD + Lipids | 7 sec/750 μl at 60° C., evaporate 2 h |
| Fe8 | F127 5% + 0.5% | Homogenise for 2 min, ethanol injection |
| | CBD + Lipids | 7 sec/750 μl at 60° C., evaporate 2 h, |
| | | homogenise for 2 min |
| Fe2 | F127 1% + 0.5% | Homogenise for 2 min, ethanol injection |
| | CBD + Lipids | 7 sec/750 μl at 60° C., evaporate 2 h, |
| | | homogenise for 2 min |
| Fe2 | F127 1% + 0.5% | Homogenise for 2 min, ethanol injection |
| | CBD + Lipids | 7 sec/750 μl at 60° C., evaporate 2 h, |
| | | homogenise for 2 min |

### Example 17. Visual Characterization of Formulations Fd5, Fe5, Fe8, and Fe2

The formulations of Table 21 were inspected visually for transparency and presence of precipitates (Table 22). All formulations were transparent and showed only a hint of white cloudiness. Formulations Fd5 had some precipitates. Formulations Fe5 and Fe8 showed no visually observable precipitates. Formulations Fe2 showed the highest level of visually observable precipitates compared to formulations Fd5, Fe5, and Fe8.

TABLE 22
Visual observations of formulations Fd5, Fe5, Fe8, and Fe2

| Formulation No. | Visual Observations |
|---|---|
| Fd5 | Transparent with a hint of white, some precipitate |
| Fd5 | Transparent with a hint of white, some precipitate |
| Fe5 | Transparent with a hint of white, no precipitate |
| Fe5 | Transparent with a hint of white, no precipitate |
| Fe8 | Transparent with a hint of white, no precipitate |
| Fe8 | Transparent with a hint of white, no precipitate |
| Fe2 | Transparent with a hint of white, precipitate |
| Fe2 | Transparent with a hint of white, precipitate |

### Example 18. Effect of Filtration on CBD Lipid-Polymer Nanoparticle Size and pH

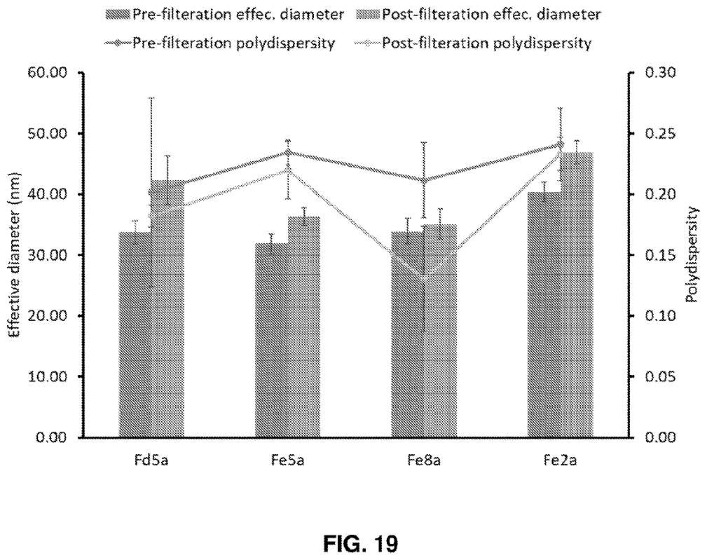

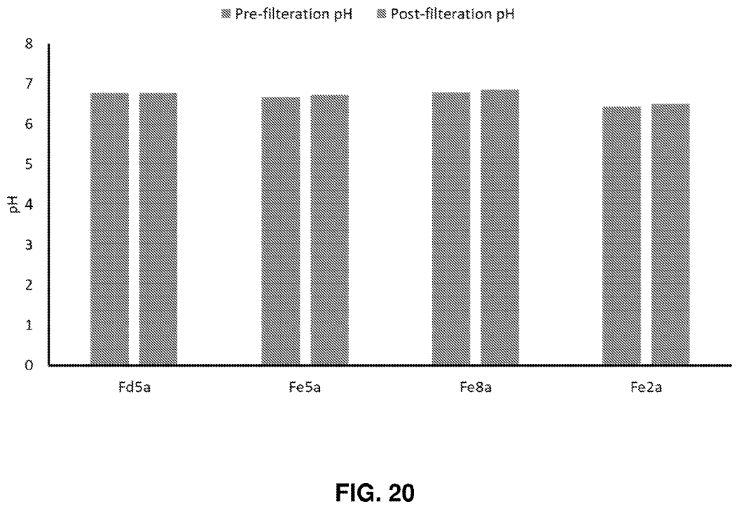

Pressure-driven filtration of lipid-polymer suspensions using non-pyrogenic, PES membrane, with a pore size of 0.22 μm was performed and the diameter of the nanoparticles was compared before and after filtration (FIG. 19). The polydispersity of the lipid-polymer nanoparticles was also characterized. Formulations Fd5a, Fe5a, Fe8a, and Fe2a all showed an increase in effective diameter following filtration (FIG. 19). The polydispersity of nanoparticles from all formulations (Fd5a, Fe5a, Fe8a, and Fe2a) decreased following filtration. Filtration of the suspensions did not produce an observable difference in the pH (FIG. 20).

### Example 19. Effect of Injection of Lipids at 45° C. on Nanoparticle Size, Polydispersity, and Visual Appearance

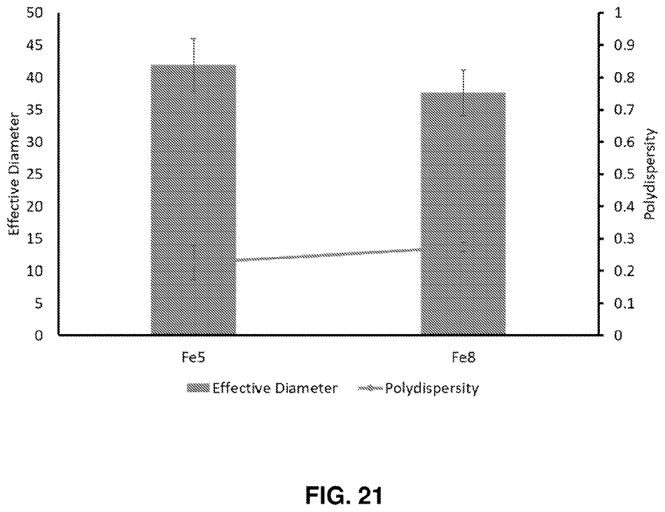

Lipid-polymer nanoparticles of formulations Fe5 and Fe8 were prepared using an injection of lipids (DSPC and cholesterol) dissolved in ethanol. In this experiment the injection was performed at 45° C. Nanoparticle size and polydispersity was characterized (FIG. 21). The effective diameter of nanoparticles of formulation Fe5 following lipid injection at 45° C. was observed to be larger than the effective diameter of the nanoparticles of formulation Fe8 following lipid injection at 45° C. Polydispersity was similar between the two formulations (FIG. 21). The visual appearance of formulation Fe5 was characterized to be transparent with a hint of white and with no observable precipitates.

### Example 20. Formulations of CBD Lipid-Polymer Nanoparticles Using Sodium Hyaluronate

The glycosaminoglycan sodium hyaluronate (SH) is known to aid in the absorption of topical pharmaceuticals. We incorporated sodium hyaluronate into the previously optimized formulations, Fe5 and Fe8, at a concentration of 0.15% w/v. The solution was sonicated after the addition of sodium hyaluronate and before the addition of CBD. Formulations containing sodium hyaluronate (Table 23) where then characterized.

TABLE 23
Formulations containing sodium hyaluronate

| Formulation No. | Formulation |
|---|---|
| Ff2 | Fe5 + 0.15% Sodium Hyaluronate |
| Ff3 | Fe8 + 0.15% Sodium Hyaluronate |

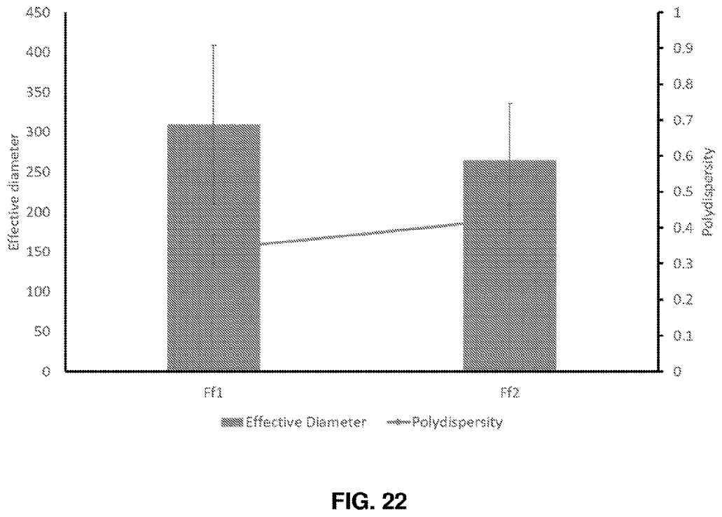

The nanoparticle size for formulations Ff1 and Ff2 was measured along with the polydispersity (FIG. 22). The visual appearance of Ff2 and Ff3 was transparent with a hint of white and no precipitate was observed (Table 24).

TABLE 24
Visual appearance of formulations containing sodium hyaluronate

| Formulation | |
|---|---|
| No. | Visual Observations |
| Ff2 | Transparent with a hint of white (more cloudy than Fe5), |
| | No precipitate, SH not fully dissolved |
| Ff3 | Transparent with a hint of white (more cloudy than Fe8), |
| | No precipitate, SH not fully dissolved |

### Example 20. Effect of Temperature and Humidity on Suspension Stability

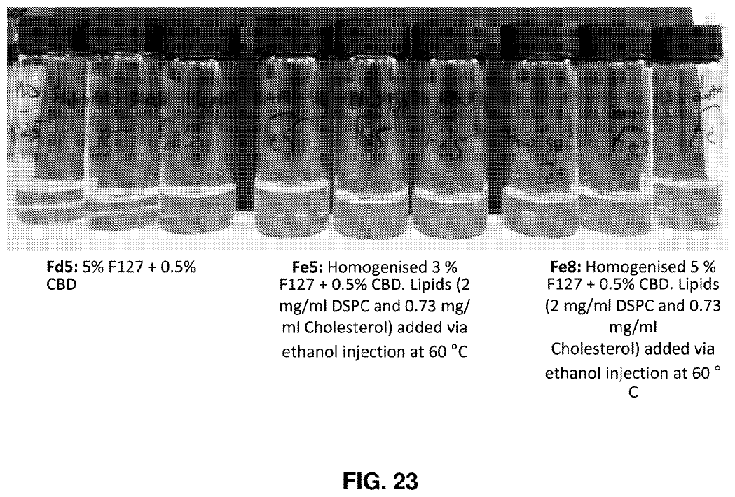

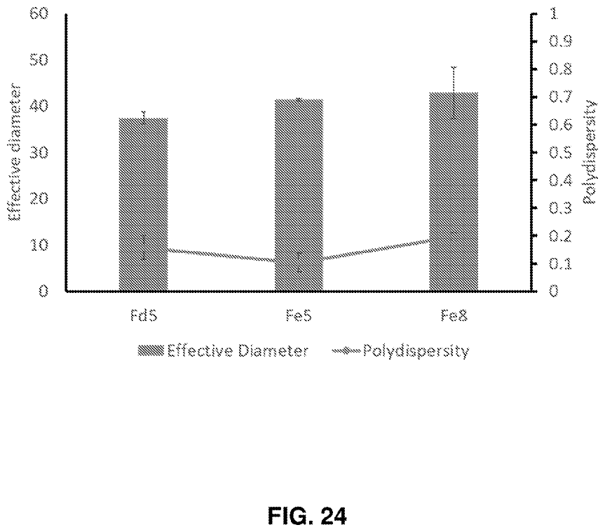

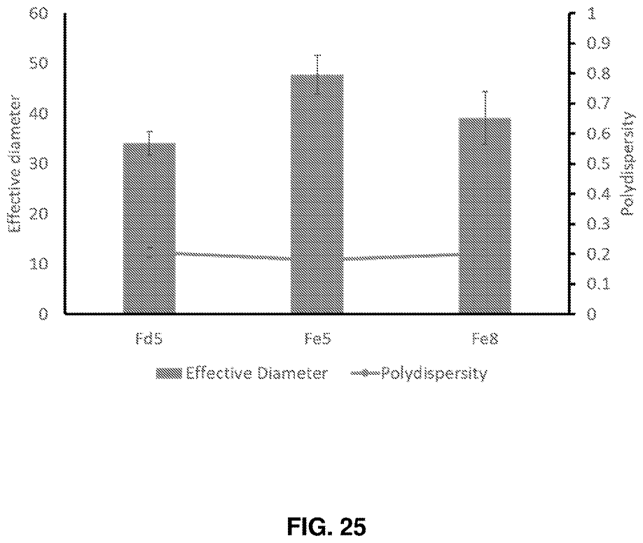

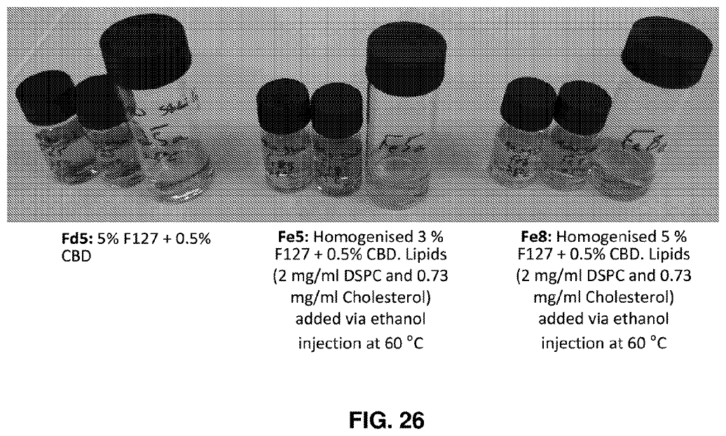

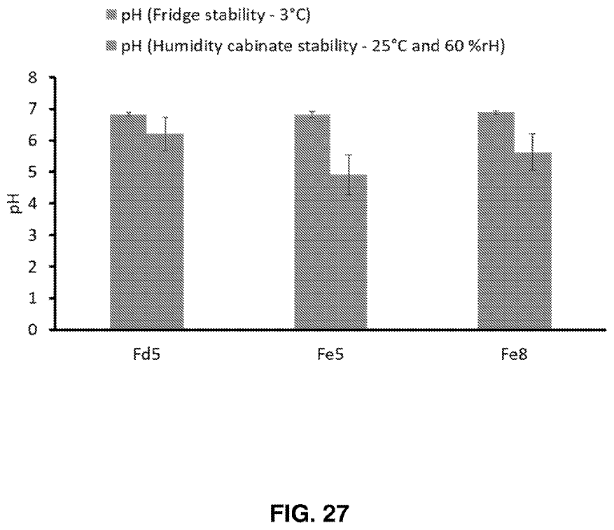

Suspension stability was characterized for formulations Fd5, Fe5, and Fe8. Suspensions were either stored at 3° C. or at 25° C. and 60% relative humidity (rH). Samples stored at 3° C. (FIG. 23) for 117 days did not demonstrate changes in appearance or size and polydispersity (FIG. 24). Samples stored at 25° C. and 60% relative humidity showed no significant change in size and polydispersity (FIG. 25) but did change in color (FIG. 26) and pH (FIG. 27) after 132 days from preparation.

### Example 21. Incorporation of Antioxidants to the Formulations

To improve the stability of the Fd5, Fe5, and Fe8 formulations we added citric acid or sodium sulfite as antioxidants to the formulations. Citric acid anhydrous (0.05% w/v) or sodium sulfite (0.2% w/v) were added to the formulation after the ethanol was evaporated. The formulation was then stirred for 10-25 minutes until the antioxidant was completely dissolved. Upon visual inspection (Table 25) there was no apparent change in the appearance of the suspensions. Formulation Fd5 with sodium sulfite showed a slight color change to a pink color after a few days of preparation.

TABLE 25
Visual appearance of Fd5, Fe5, and Fe8 with antioxidants

| Formulation No. | Observations |
|---|---|
| Fd5 + Citric acid anhydrous | No apparent change |
| Fe5 + Citric acid anhydrous | No apparent change |
| Fe8 + Citric acid anhydrous | No apparent change |
| Fd5 + sodium sulfite | No apparent change (gained an increasing |
| | pink colour after a few days) |
| Fe5 + sodium sulfite | No apparent change |
| Fe8 + sodium sulfite | No apparent change |

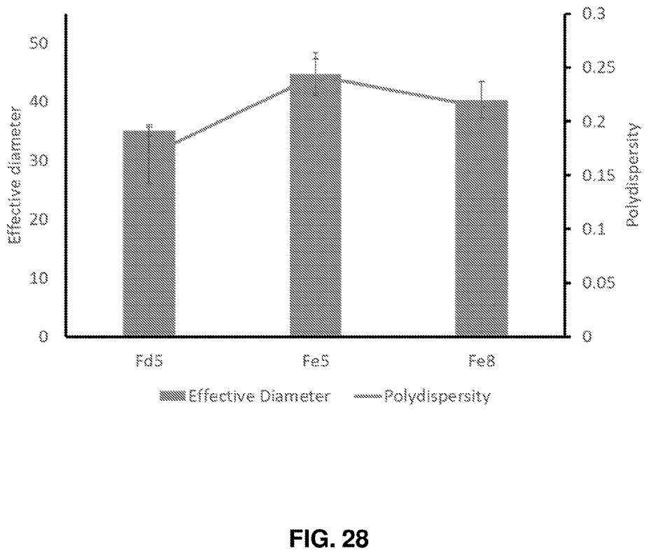

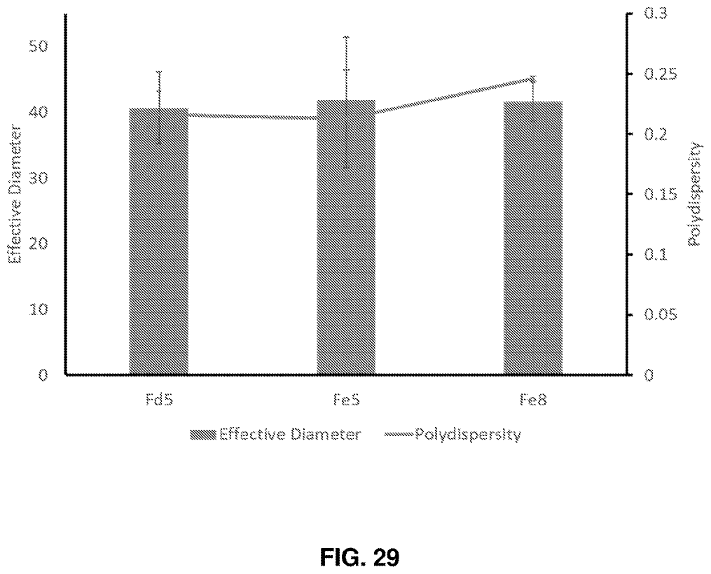

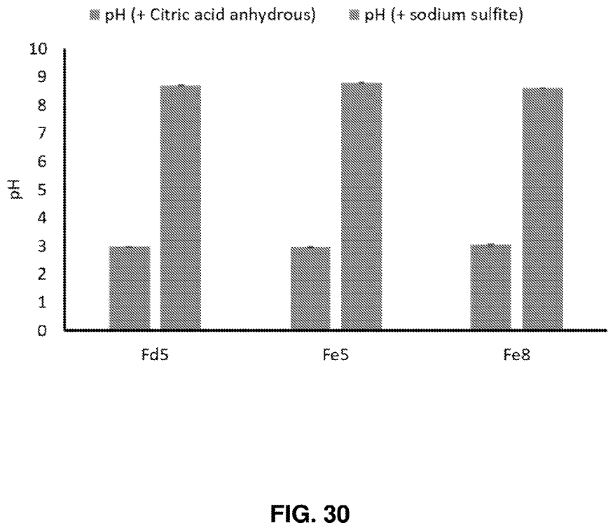

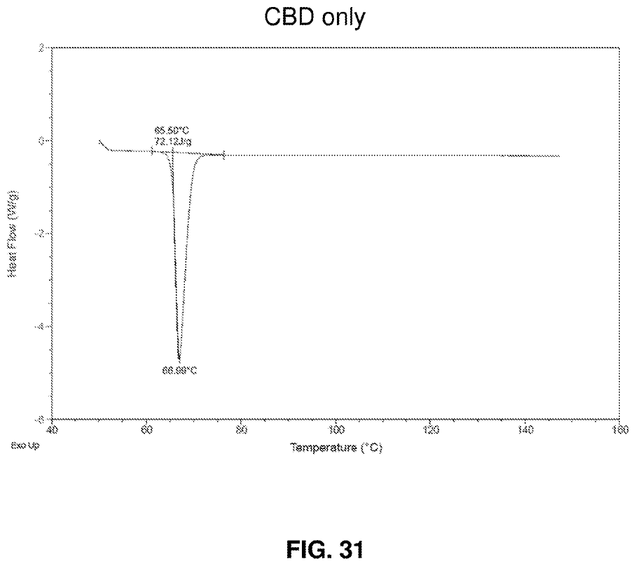

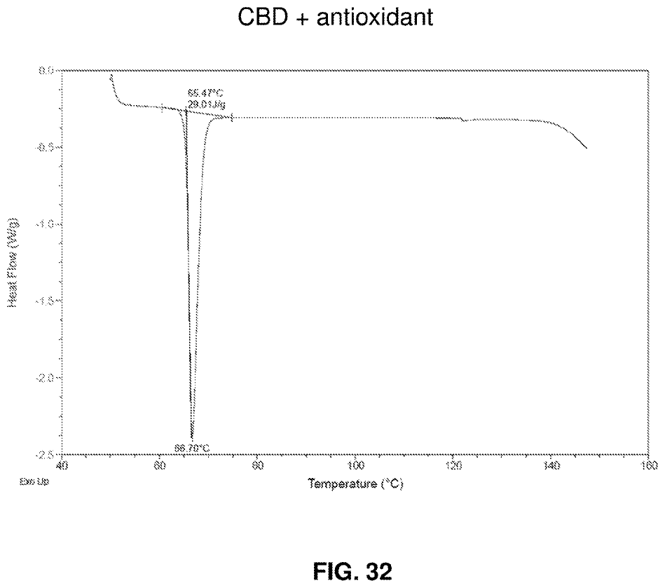

The size, polydispersity and pH were also characterized for formulations Fd5, Fe5, and Fe8 (FIGS. 28-30). To address compatibility of citric acid and sodium sulfite with CBD we also measured melting point temperatures of the formulations of CBD only (FIG. 31) and CBD+antioxidant (FIG. 32). The mean melting point for the CBD only formulations was measured to be 65.43±0.21 whereas the mean melting point for the formulations containing either citric acid or sodium sulfite was measured to be 65.37±0.14. No significant change in melting point was observed when combining CBD with the antioxidants citric acid or sodium sulfite demonstrating compatibility with CBD.

### Enumerated Embodiments

1. A pharmaceutical composition for the treatment or prevention of an ophthalmic condition, comprising therapeutically effective amounts of at least one cannabinoid (e.g., cannabidiol or derivatives thereof), and a pharmaceutically acceptable excipient or carrier, wherein the cannabinoid is present in a concentration between about 0.01% to about 10% by weight of the composition.

2. The pharmaceutical composition of embodiment 1, wherein the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

3. The pharmaceutical composition of embodiments 1 or 2, further comprising one or more terpenes selected from the group consisting of myrcene, beta-caryophyllene, linalool, alpha pinene, beta-pinene, ocimene, terpinolene, ocimene, terpinolene, alpha terpineol, alpha terpinene, gamma terpinene, alpha phellandrene, cymene, camphene, delta-3-carene, fenchol, 1,8-cineole, nerolidol, borneol, eucalyptol, camphene, and limonene, wherein one or more terpene is present at a concentration between about 0.01% to about 10% by weight of the composition.

4. The pharmaceutical composition of any one of embodiments 1-3, further comprising one or more flavonoid selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, isoflavonoids, and a combination of one or more flavonoid, wherein one or more flavonoid is present at a concentration between about 0.01% to about 10% by weight of the composition.

5. The pharmaceutical composition of any one of embodiments 1-4, further comprising β-sitosterol, wherein the β-sitosterol is present at a concentration between about 0.01% to about 10% by weight of the composition.

6. The pharmaceutical composition of any one of embodiments 1-5, further comprising methyl salicylate, wherein the methyl salicylate is present at a concentration between about 0.01% to about 10% by weight of the composition.

7. The pharmaceutical composition of any one of embodiments 1-6, further comprising tocopherol, selected from the group consisting of alpha tocopherol, gamma tocopherol, and a combination of alpha and gamma tocopherol, wherein the tocopherol is present in a concentration between about 0.01% to about 10% by weight of the composition.

8. The pharmaceutical composition of any one of embodiments 1-7, further comprising alkaloids, lignans, or a combination of alkaloids and lignans.

9. The pharmaceutical composition of any one of embodiments 1-8, further comprising one or more active agents selected from the group consisting of an antibiotic, an antiseptic agent, an antifungal, an antibacterial agent, an analgesic, non-steroidal anti-inflammatory agent, antiprotozoal agent, steroid, and an antiviral agent.

10. The pharmaceutical composition of any one of embodiments 1-9, further comprising, a thickening agent, a solubilizing agent, a tonicity agent, a pH adjuster, a preservative, an antioxidant, osmotic agent, micellization agent, demulcent, surfactant, or a combination thereof.

11. The pharmaceutical composition of embodiment 10, wherein the thickening agent is selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, and combinations thereof.

12. The pharmaceutical composition of any one of embodiments 10-11, wherein the solubilizing agent is selected from ethanol, glycerin, propylene glycol, polyethylene glycol, copolymers of ethylene oxide and propylene glycol, and combinations thereof.

13. The pharmaceutical composition of any one of embodiments 10-12, wherein the tonicity agent is selected from the group consisting of phosphate-buffered saline (PBS), Alsever's solution, Tris-buffered saline (TBS), water, balanced salt solutions (BSS), sodium chloride, potassium chloride, calcium chloride, magnesium chloride, mannitol, sorbitol, dextrose, glycerin, propylene glycol, ethanol, trehalose, and combinations thereof.

14. The pharmaceutical composition of any one of embodiments 10-13, wherein the preservative is selected from the group consisting of benzalkonium chloride (BAK), cetrimonium, sodium perborate, ethylenediaminetetraacetic acid (EDTA), chlorobutanol, and combinations thereof.

15. The pharmaceutical composition of any one of embodiments 10-14, wherein the antioxidant is selected from the group consisting of vitamin E, carnosine, N-acetylcarnosine, pyruvate, resveratrol, astaxanthin, glutathione, cysteine, cysteine ascorbate, and combinations thereof.

16. The pharmaceutical composition of any one of embodiments 1-15, wherein the composition is encapsulated in liposomes.

17. The pharmaceutical composition of any one of embodiments 1-16, wherein the composition comprises a liquid formulation suitable for use as an eye drop.

18. The pharmaceutical composition of embodiment 17, wherein the composition is an aqueous solution comprising at least one water soluble cannabinoid and saline.

19. The pharmaceutical composition of any one of embodiments 1-16, wherein the composition is coated on nanoparticles.

20. The pharmaceutical composition of any one of embodiments 1-16, wherein the composition is formulated as a gel, a thin film, an ointment, non-aqueous solution, a solid form, a paste, a liquid, an aerosol, a mist, a polymer, a film, an emulsion, a suspension, or an injectable formulation.

21. The pharmaceutical composition of embodiment 1, wherein the cannabinoid is present in a concentration of about 3% (w/w), suspended in a non-allergic pharmaceutically acceptable carrier comprising white petrolatum at a concentration of about 50% (w/w), mineral oil at a concentration of about 40% (w/w), and lanolin at a concentration of about 2% (w/w).

22. The pharmaceutical composition of any one of embodiments 1-16, wherein the composition is coated on punctal plugs.

23. The pharmaceutical composition of embodiment 22, wherein the punctal plugs are coated or impregnated with nanoparticles comprising the pharmaceutical composition.

24. A pharmaceutical eluting contact lens comprising a contact lens coated or embedded with the pharmaceutical composition of any one of embodiments 1-16, wherein the pharmaceutical composition is released from the contact lens when the lens is placed on an individual's eye.

25. A method of treating an ophthalmic condition of an individual, comprising: a) providing the pharmaceutical composition of any one of embodiments 1-23; and b) administering a therapeutically effective amount of the pharmaceutical composition to an eye of an individual suspected of having an ophthalmic condition.

26. The method of embodiment 25, wherein the pharmaceutical composition is in the form of an eye drop, and wherein administering a therapeutically effective amount of the pharmaceutical composition comprises applying the eye drop to the eye of the individual.

27. The method of embodiment 26, wherein the pharmaceutical composition is in the form of an ointment, and wherein administering a therapeutically effective amount of the pharmaceutical composition comprises applying the ointment to the eye of the individual.

28. The method of embodiment 24, wherein the pharmaceutical composition is in the form of a contact lens coated or embedded with the pharmaceutical composition, and wherein administering a therapeutically effective amount of the pharmaceutical composition comprises placing the contact lens on the individual's eye, wherein the therapeutically effective amount of the pharmaceutical composition is released from the contact lens.

29. The method of embodiment 24, wherein the pharmaceutical composition is in the form of a punctal plug coated with the pharmaceutical composition, and wherein administering a therapeutically effective amount of the pharmaceutical composition comprises placing the punctal plug on the individual's eye, wherein the therapeutically effective amount of the pharmaceutical composition is released from the coated punctal plug.

30. The method of any one of embodiments 24-29, wherein the ophthalmic condition is keratitis.

31. The method of embodiment 30, wherein the keratitis is bacterial keratitis, protozoal keratitis, fungal keratitis, or viral keratitis.

32. The method of any one of embodiments 24-29, wherein the ophthalmic condition is conjunctivitis.

33. The method of embodiment 32, wherein the conjunctivitis is bacterial conjunctivitis or viral conjunctivitis.

34. The method of any one of embodiments 24-29, wherein the ophthalmic condition is episcleritis or scleritis.

35. The method of any one of embodiments 24-29, wherein the ophthalmic condition is corneal abrasion.

36. The method of any one of embodiments 24-29, wherein the ophthalmic condition is inflammation of the eye.

37. The method of any one of embodiments 24-29 wherein the ophthalmic condition is injury to the eye following eye surgery.

38. The method of any one of embodiments 24-29, wherein the ophthalmic condition is blepharoconjunctivitis.

39. The method of any one of embodiments 24-29, wherein the ophthalmic condition is ocular rosacea.

40. The method of any one of embodiments 24-29, wherein the ophthalmic condition is glaucoma.

41. A method for treating a human suffering from dry eye, comprising: a) providing the pharmaceutical composition of any one of embodiments 1-23; and b) administering a therapeutically effective amount of the pharmaceutical composition to an eye of the human suffering from dry eye.

42. A method for treating a human suffering from meibomianitis, comprising: a) providing the pharmaceutical composition of any one of embodiments 1-23; and b) administering a therapeutically effective amount of the pharmaceutical composition to an eye or an area surrounding the eye of the human suffering from meibomianitis.

43. The method of embodiment 42, wherein the pharmaceutical composition is a liquid ophthalmic composition.

44. The method of embodiment 43, wherein the liquid ophthalmic composition is selected from the group consisting of a solution, a suspension, an emulsion, and an in-situ gel system.

45. The method of embodiment 42, wherein the pharmaceutical composition is a solid ophthalmic composition selected from the group consisting of a gel and an ointment, wherein the pharmaceutical composition is administered to an eyelid of the human.

46. A method for treating a human suffering from blepharitis, comprising: a) providing the pharmaceutical composition of any one of embodiments 1-23; and b) administering a therapeutically effective amount of the pharmaceutical composition to an eye of the human suffering from blepharitis.

47. The method of embodiment 46, wherein the pharmaceutical composition is a liquid ophthalmic composition.

48. The method of embodiment 47, wherein the liquid ophthalmic composition is selected from the group consisting of a solution, a suspension, an emulsion, and an in-situ gel system.

49. The method of embodiment 46, wherein the pharmaceutical composition is a solid ophthalmic composition selected from the group consisting of a gel and an ointment, wherein the pharmaceutical composition is administered to an eyelid of the human.

50. A kit for treating an ophthalmic condition, comprising: a) a first ophthalmic formulation containing a cannabinoid or derivatives thereof for administration during daytime; and b) a second ophthalmic formulation containing a cannabinoid or derivatives thereof for administration prior to sleep, wherein the second ophthalmic formulation has a higher viscosity than the first ophthalmic formulation.

51. The kit of embodiment 50, wherein the first and the second ophthalmic formulation contains a cannabinoid or derivative thereof in a concentration between about 0.01% to about 10% by weight.

52. The kit of embodiment 50, wherein the first ophthalmic formulation is an eye drop.

53. The kit of embodiment 50, wherein the eye drop is selected from the group consisting of a solution, a suspension, and an emulsion.

54. The kit of embodiment 50, wherein the second ophthalmic formulation is selected from the group consisting of an in-situ gel, a gel, and an ointment.

55. A method for treating an ophthalmic condition of an individual, comprising: a) administering a first ophthalmic formulation to an eye of the individual, wherein the first ophthalmic formulation is a liquid ophthalmic formulation comprising at least one cannabinoid or derivatives thereof in a concentration between about 0.01% to about 10% by weight of the composition, and a pharmaceutically acceptable excipient or carrier; and b) administering a second ophthalmic formulation to an eye, wherein the second ophthalmic formulation comprises at least one cannabinoid or derivatives thereof in a concentration between about 0.01% to about 10% by weight of the composition, and appropriate pharmaceutically acceptable excipients or carriers, and wherein the second ophthalmic formulation has a higher viscosity than the first ophthalmic formulation.

56. The method of embodiment 55, further comprising administering the second ophthalmic formulation to an area surrounding the eye.

57. The method of embodiment 55, further comprising administering the second ophthalmic formulation to the eyelid.

58. The method of embodiment 55, wherein the first ophthalmic formulation is selected from the group consisting of a solution, a suspension, and an emulsion.

59. The method of embodiment 55, wherein the second ophthalmic formulation is selected from the group consisting of an in-situ forming gel, a gel, and an ointment.

60. The method of embodiment 55, wherein the second ophthalmic formulation is administered at about bedtime.

61. The method of embodiment 55, wherein the first ophthalmic formulation is administered during daytime or during daylight hours.

62. The method of embodiment 55, wherein the first ophthalmic formulation is administered at a dosing schedule selected from the group consisting of once per day, twice per day, three times per day, four times per day, five times per day, six times per day, seven times per day, eight times per day, nine times per day, ten times per day, eleven times per day, twelve times per day, and hourly.

63. The pharmaceutical composition of any one of embodiments 1-8, wherein the composition further comprises Manuka honey.

64. A pharmaceutical composition comprising therapeutically effective amounts of: (a) a cannabinoid; and (b) a parasympathetic agonist.

65. The pharmaceutical composition of embodiment 64, further comprising an active agent selected from the group consisting of a sympathetic antagonist, a sympathetic agonist, and combinations thereof.

66. The pharmaceutical composition of embodiment 65, wherein the active agent is a sympathetic antagonist.

67. The pharmaceutical composition of embodiment 65, wherein the active agent is a combination of a sympathetic agonist and a sympathetic antagonist.

68. The pharmaceutical composition of any one of embodiments 65-67, wherein the sympathetic antagonist is an alpha-adrenergic blocking agent such as dapiprazole or thymoxamine.

69. The pharmaceutical composition of any one of embodiments 64-68, wherein the cannabinoid is a cannabidiol.

70. The pharmaceutical composition of any one of embodiments 65 and 67, wherein the active agent is a sympathetic agonist.

71. The pharmaceutical composition of embodiments 64 and 70, wherein the cannabinoid is CBD-1, wherein the parasympathetic agonist is pilocarpine, and the sympathetic agonist is brimonidine.

72. The pharmaceutical composition of any one of embodiments 64-71, wherein the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

73. The pharmaceutical composition of any one of embodiments 64-72 wherein the parasympathetic agonist is a cholinergic agonist.

74. The pharmaceutical composition of any one of embodiments 64-72, wherein the parasympathetic agonist is selected from the group consisting of bethanechol, carbamylcholine, cevimeline, and pilocarpine.

75. The pharmaceutical composition of any one of embodiments 64-72, wherein the parasympathetic agonist is a cholinesterase inhibitor.

76. The pharmaceutical composition of embodiment 75, wherein the cholinesterase inhibitor is selected from the group consisting of delta-9-tetrahydrocannabinol, carbamates, physostigmine, neostigmine, pyridostigmine, ambenonium, demecarium, rivastigmine, phenanthrene derivatives, galantamine, caffeine—noncompetitive, piperidines, donepezil, tacrine, edrophonium, huperzine, ladostigil, ungeremine and lactucopicrin.

77. The pharmaceutical composition of any one of embodiments 65, 67, 69-76, wherein the sympathetic agonist is selected from the group consisting of brimonidine, clonidine, guanfacine, guanabenz, guanoxabenz, guanethidine, iopidine, tizanidine, and xylazine.

78. The pharmaceutical composition of any one of embodiments 65, 67, 69-76, wherein the sympathetic agonist is an adrenergic agonist.

79. The pharmaceutical composition of embodiment 78, wherein the adrenergic agonist is a α2 adrenergic agonist.

80. The pharmaceutical composition of any one of embodiments 64-79, further comprising one or more terpenes selected from the group consisting of myrcene, beta-caryophyllene, linalool, alpha pinene, beta-pinene, ocimene, terpinolene, ocimene, terpinolene, alpha terpineol, alpha terpinene, gamma terpinene, alpha phellandrene, cymene, camphene, delta-3-carene, fenchol, 1,8-cineole, nerolidol, borneol, eucalyptol, camphene, and limonene, wherein one or more terpene is present at a concentration between about 0.01% to about 10% by weight of the composition.

81. The pharmaceutical composition of any one of embodiments 64-80, further comprising one or more flavonoid selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, isoflavonoids, and a combination of one or more flavonoid, wherein one or more flavonoid is present at a concentration between about 0.01% to about 10% by weight of the composition.

82. The pharmaceutical composition of any one of embodiments 64-81, further comprising a solubilizing agent is optionally selected from ethanol, glycerin, propylene glycol, polyethylene glycol, copolymers of ethylene oxide and propylene glycol, and combinations thereof.

83. The pharmaceutical composition of any one of embodiments 64-82, wherein the pharmaceutical composition is formulated for application to the eye.

84. The pharmaceutical composition of any one of embodiments 64-83, wherein the composition comprises a liquid formulation suitable for use as an eye drop.

85. The pharmaceutical composition of any one of embodiments 64-83, wherein the composition comprises CBD-1, pilocarpine, and brimonidine.

86. A method of treating a vision disorder comprising: a) providing the pharmaceutical composition of any one of embodiments 64-85; and b) administering a therapeutically effective amount of the pharmaceutical composition to an eye of a subject identified as having a vision disorder.

87. The method of embodiment 86, wherein the pharmaceutical composition is in the form of an eye drop or an ointment.

88. The method of any one of embodiments 86-87, wherein the vision disorder is selected from the group consisting of presbyopia, hypermetropia, and astigmatism.

89. The method of any one of embodiments 86-88, wherein the pharmaceutical composition is administered to the eye of the subject at least twice per day during a course of treatment.

90. The method of any one of embodiments 86-89, wherein the pharmaceutical composition is administered to the eye of the subject at least once a day during a course of treatment.

91. The method of any one of embodiments 86-89, wherein the pharmaceutical composition is administered to the eye of the subject at least once every two days during a course of treatment.

92. The method of any one of embodiments 86-91, wherein the pharmaceutical composition is administered to the eye of the subject at a dosing interval selected from the group consisting of four times a day, three times a day, twice a day, once a day, once every two days, once every three days, and once every week.

93. The method of any one of embodiments 86-89, wherein the pharmaceutical composition is administered each day for at least seven days.

94. A topical composition for the treatment or prevention of a dermatological condition, comprising a therapeutically effective amount of at least one cannabidiol, its analogs, derivatives, or a combination thereof, wherein the cannabidiol, its analogs, derivatives, or a combination thereof is present in a concentration between about 0.001% to about 10% by weight of the composition, and appropriate topical pharmaceutically acceptable excipients or carriers.

95. The topical composition of embodiment 94, wherein the therapeutically effective amount of the cannabidiol, its analogs, derivatives, or a combination thereof is about 0.01% to about 4% by weight of the composition.

96. The topical composition of embodiment 95, wherein the therapeutically effective amount of the cannabidiol, its analogs, derivatives, or a combination thereof is about 0.1% to about 3% by weight of the composition.

97. The topical composition of any one of embodiments 94-96, the cannabidiol, its analogs, derivatives, or a combination thereof is one or more of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

98. The topical composition of any one of embodiments 94-97, further comprising one or more terpenes selected from the group consisting of myrcene, beta-caryophyllene, linalool, alpha pinene, beta-pinene, ocimene, terpinolene, ocimene, terpinolene, alpha terpinol, alpha terpinene, gamma terpinene, alpha phellandrene, cymene, camphene, delta-3-carene, fenchol, 1,8-cineole, nerolidol, borneol, eucalyptol, camphene, and limonene.

99. The topical composition of embodiment 98, wherein one or more terpene is present in a concentration between about 0.01% to about 10% by weight of the composition.

100. The topical composition of any one of embodiments 94-99, further comprising one or more flavonoid is selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, isoflavonoids, and a combination of one or more flavonoid, wherein one or more flavonoid is present in a concentration between about 0.01% to about 10% by weight of the composition.

101. The topical composition of any one of embodiments 94-100, further comprising β-sitosterol, wherein the β-sitosterol is present in a concentration between about 0.01% to about 10% by weight of the composition.

102. The topical composition of any one of embodiments 94-101, further comprising methyl salicylate, wherein the methyl salicylate is present in a concentration between about 0.01% to about 10% by weight of the composition.

103. The topical composition of any one of embodiments 94-102, further comprising tocopherol, selected from the group consisting of alpha tocopherol, gamma tocopherol, and a combination of alpha and gamma tocopherol, wherein the tocopherol is present in a concentration between about 0.01% to about 20% by weight of the composition.

104. The topical composition of any one of embodiments 94-103, further comprising alkaloids, lignans, or a combination of alkaloids and lignans.

105. The topical composition of any one of embodiments 94-104, further comprising one or more active agents selected from the group consisting of an antibiotic, an antiseptic agent, antiprotozoal, an antifungal, an antibacterial agent, an analgesic and an antiviral agent.

106. The topical composition of any one of embodiments 94-105, further comprising, a thickening agent, a solubilizing agent, a tonicity agent, a pH adjuster, a preservative, an antioxidant, osmotic agent, micellization agent, demulcent, surfactant, a co-surfactant, a penetration enhancer, a chelating agent, or a combination thereof.

107. The topical composition of embodiment 106, wherein the thickening agent is selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, cellulose, chitosan, and combinations thereof.

108. The topical composition of any one of embodiments 106-107, wherein the solubilizing agent is selected from ethanol, glycerin, propylene glycol, polyethylene glycol, copolymers of ethylene oxide and propylene glycol, and combinations thereof.

109. The topical composition of any one of embodiments 106-108, wherein the tonicity agent is selected from the group consisting of phosphate-buffered saline (PBS), Alsever's solution, Tris-buffered saline (TBS), water, balanced salt solutions (BSS), sodium chloride, potassium chloride, calcium chloride, magnesium chloride, mannitol, sorbitol, dextrose, glycerin, propylene glycol, ethanol, trehalose, and combinations thereof.

110. The topical composition of any one of embodiments 106-109, wherein the preservative is selected from the group consisting of benzalkonium chloride (BAK), cetrimonium, sodium perborate, EDTA, chlorobutanol, and combinations thereof.

111. The topical composition of any one of embodiments 106-110, wherein the antioxidant is selected from the group consisting of vitamin E, carnosine, N-acetylcarnosine, pyruvate, resveratrol, astaxanthin, glutathione, cysteine, cysteine ascorbate, and combinations thereof.

112. The topical composition of any one of embodiments 94-111, wherein a pharmaceutically acceptable carrier comprises a hydrophobic or lipophilic substance comprising a paraffin oil, mineral oil, petrolatum, bee wax, butylhydroxytoluene, liquid lanolin, propylene carbonate, an ester of a C8-C18 organic acid, a C8-C30 fatty alcohol, a silicone oil, a vegetable oil, a fractionated or hydrogenated vegetable oil, a monoglyceride, a diglyceride, a triglyceride, a phospholipid, dimethyl isosorbide, a volatile solvent, N-methylpyrrolidone, dimethylacetamide, dimethylformamide; dimethyl sulfoxide, or a combination thereof.

113. The topical composition of any one of embodiments 94-112, wherein the composition is encapsulated in liposomes, micelles, noisome, fullerene, nanoshell, quantum dot, dendrimer, lipid-polymer nanoparticles, or any combination thereof.

114. The topical pharmaceutical composition of any one of embodiments 94-112, wherein the composition is coated on nanoparticles or charged polymers.

115. The topical composition of any one of embodiments 94-114, wherein the composition is formulated as a foam, cream, paste, gel, aerosol, ointment, shampoo, or lotion.

116. The topical composition of embodiment 94, wherein the cannabidiol, analogs, derivatives, or a combination thereof is present in a concentration of about 3% (w/w), suspended in a non-allergenic pharmaceutically acceptable carrier comprising white petrolatum at a concentration of about 50% (w/w), mineral oil at a concentration of about 40% (w/w), and lanolin at a concentration of about 2% (w/w).

117. A method of treating a dermatological condition of a subject, comprising: a) providing the topical composition of any one of embodiments 94-116; and b) topically applying a therapeutically effective amount of the topical composition to the subject's skin suspected of having a dermatological condition.

118. The method of embodiment 117, wherein the dermatological condition is meibomianitis or blepharitis, and wherein topically applying the composition to the subject's skin comprises applying the composition to the outer portion of the eye lid of the individual.

119. The method of embodiment 117, wherein the dermatological condition is eczema.

120. The method of embodiment 117, wherein the dermatological condition is dermatitis.

121. The method of embodiment 120, wherein the dermatitis is atopic dermatitis, contact dermatitis, seborrheic dermatitis, perioral dermatitis, or xerotic eczema.

122. The method of embodiment 117, wherein the dermatological condition is psoriasis.

123. The method of embodiment 117, wherein the dermatological condition is acne vulgaris.

124. The method of embodiment 117, wherein the dermatological condition is dryness of the skin, tinea pedis, hives, or impetigo.

125. The method of embodiment 117, wherein the dermatological condition is non-melanoma cancer.

126. The method of embodiment 117, wherein the dermatological condition is itching dermatosis.

127. The method of embodiment 117, wherein the dermatological condition is acne rosacea.

128. The method of embodiment 117, wherein the dermatological condition is aging of skin or dandruff.

129. A pharmaceutical composition for the treatment or prevention of bacterial vaginosis, comprising therapeutically effective amounts of at least one cannabinoid or derivatives thereof, and a pharmaceutically acceptable excipient or carrier, wherein the cannabinoid is present in a concentration between about 0.01% to about 30% by weight of the composition.

130. The pharmaceutical composition of embodiment 129, wherein the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

131. The pharmaceutical composition of embodiment 129, further comprising, a thickening agent, a solubilizing agent, a pH adjuster, a preservative, surfactant, or a combination thereof.

132. The pharmaceutical composition of embodiment 131, wherein the thickening agent is selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, and combinations thereof.

133. The pharmaceutical composition of embodiment 131, wherein the preservative is selected from the group consisting of benzalkonium chloride (BAK), cetrimonium, sodium perborate, EDTA, chlorobutanol, and combinations thereof.

134. The pharmaceutical composition of embodiment 129, wherein the composition is a cream.

135. The pharmaceutical composition of embodiment 129, wherein the composition is an ointment.

136. The pharmaceutical composition of embodiment 129, wherein the composition is a gel.

137. The pharmaceutical composition of embodiment 129, wherein the composition is a suppository.

138. The pharmaceutical composition of embodiment 129, wherein the composition is a capsule.

139. The pharmaceutical composition of embodiment 129, wherein the composition is non-flowing.

140. The pharmaceutical composition of embodiment 129, wherein the cannabinoid is present in a concentration of between about 0.1% and about 30% (w/w).

141. The pharmaceutical composition of embodiment 129, wherein the composition further comprises an antibiotic.

142. The pharmaceutical composition of embodiment 141, wherein antibiotic is selected from the group consisting of metronidazole, clindamycin, tinidazole, tetracycline, amoxicillin, ampicillin, lumefloxacin, norfloxacin, afloxam, ciproflaxin, azithromycin and cefltoxine.

143. The pharmaceutical composition of embodiment 129, wherein the composition further comprises a steroid.

144. The pharmaceutical composition of embodiment 143, wherein the steroid is selected from the group consisting of hydrocortisone, cortisone acetate, prednisone, prednisolone, methylprednisolone, dexamethasone, betamethasone, triamcinolone, and beclomethasone, and fluorometholone.

145. A method of treating an individual having bacterial vaginosis, comprising: a) providing the pharmaceutical composition comprising a therapeutically effective amount of at least one cannabinoid or derivatives thereof and pharmaceutically acceptable excipients; and b) administering a therapeutically effective amount of the pharmaceutical composition to a vulvovaginal surface of the individual.

146. The method of embodiment 145, wherein the pharmaceutical composition is in a form selected from the group consisting of a cream, an ointment, a gel, a suppository, and a capsule.

147. The method of embodiment 145, wherein the pharmaceutical composition is in the form of an ointment, gel, or cream, and wherein administering a therapeutically effective amount of the pharmaceutical composition to the vulvovaginal surface comprises applying the ointment, gel, or cream to the mucosal surfaces in the vaginal cavity of the individual.

148. The method of embodiment 145, wherein the pharmaceutical composition is in the form of an ointment, gel, or cream, and wherein administering a therapeutically effective amount of the pharmaceutical composition to the vulvovaginal surface comprises applying the ointment, gel, or cream to a non-mucosal surface of a vulva of the individual.

149. The method of embodiment 145, wherein the pharmaceutical composition is in the form of a suppository or capsule, and wherein administering a therapeutically effective amount of the pharmaceutical composition to the vulvovaginal surface comprises inserting the suppository or capsule into the vaginal cavity of the individual.

150. The method of embodiment 145, wherein the pharmaceutical composition further comprises an antibiotic.

151. The method of embodiment 150, wherein antibiotic is selected from the group consisting of metronidazole, clindamycin, tinidazole, tetracycline, amoxicillin, ampicillin, lumefloxacin, norfloxacin, afloxam, ciproflaxin, azithromycin and cefltoxine.

152. A method of treating an individual having candidiasis, comprising: a) providing the pharmaceutical composition comprising a therapeutically effective amount of at least one cannabinoid or derivatives thereof and pharmaceutically acceptable excipients; and b) administering a therapeutically effective amount of the pharmaceutical composition to a vulvovaginal surface of the individual.

153. The method of embodiment 152, wherein the pharmaceutical composition is in a form selected from the group consisting of a cream, an ointment, a gel, a suppository, and a capsule.

154. The method of embodiment 152, wherein the pharmaceutical composition is in the form of an ointment, gel, or cream, and wherein administering a therapeutically effective amount of the pharmaceutical composition to the vulvovaginal surface comprises applying the ointment, gel, or cream to the mucosal surfaces in the vaginal cavity of the individual.

155. The method of embodiment 152, wherein the pharmaceutical composition is in the form of an ointment, gel, or cream, and wherein administering a therapeutically effective amount of the pharmaceutical composition to the vulvovaginal surface comprises applying the ointment, gel, or cream to a non-mucosal surface of a vulva of the individual.

156. The method of embodiment 152, wherein the pharmaceutical composition is in the form of a suppository or capsule, and wherein administering a therapeutically effective amount of the pharmaceutical composition to the vulvovaginal surface comprises inserting the suppository or capsule into the vaginal cavity of the individual.

157. The method of embodiment 152, wherein the pharmaceutical composition includes fluconazole and a topical steroid.

158. The method of embodiment 152, wherein the pharmaceutical composition further comprises an antibiotic.

159. The method of embodiment 158, wherein antibiotic is selected from the group consisting of metronidazole, clindamycin, tinidazole, tetracycline, amoxicillin, ampicillin, lumefloxacin, norfloxacin, afloxam, ciproflaxin, azithromycin and cefltoxine.

160. A kit for treating bacterial vaginosis or candidiasis, comprising: a pessary containing or coated with a pharmaceutical composition comprising a cannabinoid or derivatives thereof; and a topical formulation comprising a cannabinoid or derivatives thereof.

161. A topical composition for lightening skin, comprising a therapeutically effective amount of a skin lightening agent and a therapeutically effective amount of at least one cannabinoid, its analogs, derivatives, or a combination thereof, wherein the cannabinoid, its analogs, derivatives, or a combination thereof is present in a concentration between about 0.001% to about 10% by weight of the composition, and appropriate topical pharmaceutically acceptable excipients or carriers.

162. The topical composition of embodiment 161, wherein the therapeutically effective amount of the cannabinoid, its analogs, derivatives, or a combination thereof is about 0.01% to about 4% by weight of the composition.

163. The topical composition of embodiment 161 wherein the therapeutically effective amount of the cannabinoid, its analogs, derivatives, or a combination thereof is about 0.1% to about 3% by weight of the composition.

164. The topical composition of any one of embodiments 161-163, the cannabinoid, its analogs, derivatives, or a combination thereof is one or more of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

165. The topical composition of any one of embodiments 161-164, wherein the skin lightening agent is selected from the group consisting of hydroquinone, L-ascorbic acid, glycolic acid, lactic acid, arbutin, kojic acid, daisy flower extract, licorice extract, and placenta extract.

166. The topical composition of any one of embodiments 161-165, wherein the skin lightening agent is an inhibitor of tyrosinase.

167. The topical composition of any one of embodiments 161-166, wherein the skin lightening agent is hydroquinone.

168. The topical composition of any one of embodiments 161-167, wherein the therapeutically effective amount of the skin lightening agent is about 4%.

169. The topical composition of any one of embodiments 161-168 wherein the therapeutically effective amount of the skin lightening agent is about 10%.

170. The topical composition of any one of embodiments 161-169, further comprising an antibiotic.

171. The topical composition of embodiment 170, wherein the antibiotic is selected from the group consisting of erythromycin, clindamycin, lymecycline, tinidazole, tetracycline, amoxicillin, ampicillin, lumefloxacin, norfloxacin, afloxam, ciproflaxin, azithromycin, and cefltoxine.

172. The topical composition of any one of embodiments 161-171 further comprising a steroid.

173. The topical composition of embodiment 172, wherein the steroid is selected from the group consisting of hydrocortisone, cortisone acetate, prednisone, prednisolone, methylprednisolone, dexamethasone, betamethasone, triamcinolone, and beclomethasone, fluorometholone. Other glucocorticoids useful in the treatment method include, but are not limited to, 21-acetoxypregnenolone, alelometasone, algestone, amcinonide, budesonide, chloroprednisone, clobetasol, clobetasone (EUMOVATE®), clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, rimexolone, tixocortol, triamcinolone acetonide, triamcinolone benetonide, triamcinolone hexacetonide, their ophthalmically acceptable salts, combinations thereof, and mixtures thereof.

174. The topical composition of any one of embodiments 161-173, further comprising a vitamin.

175. The topical composition of embodiment 174, wherein the vitamin is selected from the group consisting of vitamin A, vitamin B, vitamin C, vitamin D, and vitamin E.

176. The topical composition of any one of embodiments 161-175, further comprising an agent for treating acne.

177. The topical composition of embodiment 176, wherein the agent for treating acne is selected from the group consisting of salicylic acid, glycolic acid, or lactic acid.

178. The topical composition of any one of embodiments 161-177, further comprising an agent for treating wrinkles and/or fine lines.

179. The topical composition of embodiment 178, wherein the agent for treating wrinkles and/or fine lines is a retinoid.

180. The topical composition of any one of embodiments 161-179, further comprising one or more terpenes selected from the group consisting of myrcene, beta-caryophyllene, linalool, alpha pinene, beta-pinene, ocimene, terpinolene, ocimene, terpinolene, alpha terpinol, alpha terpinene, gamma terpinene, alpha phellandrene, cymene, camphene, delta-3-carene, fenchol, 1,8-cineole, nerolidol, borneol, eucalyptol, camphene, and limonene.

181. The topical composition of embodiment 180, wherein one or more terpene is present in a concentration between about 0.01% to about 10% by weight of the composition.

182. The topical composition of any one of embodiments 161-181, further comprising one or more flavonoid is selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, isoflavonoids, and a combination of one or more flavonoid, wherein one or more flavonoid is present in a concentration between about 0.01% to about 10% by weight of the composition.

183. The topical composition of any one of embodiments 161-182, further comprising β-sitosterol, wherein the β-sitosterol is present in a concentration between about 0.01% to about 10% by weight of the composition.

184. The topical composition of any one of embodiments 161-183, further comprising methyl salicylate, wherein the methyl salicylate is present in a concentration between about 0.01% to about 10% by weight of the composition.

185. The topical composition of any one of embodiments 161-184, further comprising tocopherol, selected from the group consisting of alpha tocopherol, gamma tocopherol, and a combination of alpha and gamma tocopherol, wherein the tocopherol is present in a concentration between about 0.01% to about 20% by weight of the composition.

186. The topical composition of any one of embodiments 161-185, further comprising alkaloids, lignans, or a combination of alkaloids and lignans.

187. The topical composition of any one of embodiments 161-186, further comprising one or more active agents selected from the group consisting of an antibiotic, an antiseptic agent, antiprotozoal, an antifungal, an antibacterial agent, an analgesic and an antiviral agent.

188. The topical composition of any one of embodiments 161-187, further comprising, a thickening agent, a solubilizing agent, a tonicity agent, a pH adjuster, a preservative, an antioxidant, osmotic agent, micellization agent, demulcent, surfactant, a co-surfactant, a penetration enhancer, a chelating agent, or a combination thereof.

189. The topical composition of embodiment 188, wherein the thickening agent is selected from hyaluronic acid, carboxymethylcellulose, hydroxypropyl cellulose, polyvinyl alcohol, polyacrylic acid, xanthan gum, guar gum, dextran, polyvinyl pyrrolidone, polyethylene glycol, cellulose, chitosan, and combinations thereof.

190. The topical composition of any one of embodiments 188-189, wherein the solubilizing agent is selected from ethanol, glycerin, propylene glycol, polyethylene glycol, copolymers of ethylene oxide and propylene glycol, and combinations thereof.

191. The topical composition of any one of embodiments 188-190, wherein the tonicity agent is selected from the group consisting of phosphate-buffered saline (PBS), Alsever's solution, Tris-buffered saline (TBS), water, balanced salt solutions (BSS), sodium chloride, potassium chloride, calcium chloride, magnesium chloride, mannitol, sorbitol, dextrose, glycerin, propylene glycol, ethanol, trehalose, and combinations thereof.

192. The topical composition of any one of embodiments 188-191, wherein the preservative is selected from the group consisting of BAK, cetrimonium, sodium perborate, EDTA, chlorobutanol, and combinations thereof.

193. The topical composition of any one of embodiments 188-192, wherein the antioxidant is selected from the group consisting of vitamin E, carnosine, N-acetylcarnosine, pyruvate, resveratrol, astaxanthin, glutathione, cysteine, cysteine ascorbate, and combinations thereof.

194. The topical composition of any one of embodiments 161-193, wherein a pharmaceutically acceptable carrier comprises a hydrophobic or lipophilic substance comprising a paraffin oil, mineral oil, petrolatum, bee wax, butylhydroxytoluene, liquid lanolin, propylene carbonate, an ester of a C8-C18 organic acid, a C8-C30 fatty alcohol, a silicone oil, a vegetable oil, a fractionated or hydrogenated vegetable oil, a monoglyceride, a diglyceride, a triglyceride, a phospholipid, dimethyl isosorbide, a volatile solvent, N-methylpyrrolidone, dimethylacetamide, dimethylformamide; dimethyl sulfoxide, or a combination thereof.

195. The topical composition of any one of embodiments 161-194, wherein the composition is encapsulated in liposomes, micelles, noisome, fullerene, nanoshell, quantum dot, dendrimer, lipid-polymer nanoparticles, or any combination thereof.

196. The topical pharmaceutical composition of any one of embodiments 161-195, wherein the composition is coated on nanoparticles or charged polymers.

197. The topical composition of any one of embodiments 161-196, wherein the composition is formulated as a foam, cream, paste, gel, aerosol, ointment, shampoo, or lotion.

198. The topical composition of embodiment 161, wherein the cannabinoid, analogs, derivatives, or a combination thereof is present in a concentration of about 3% (w/w), suspended in a non-allergenic pharmaceutically acceptable carrier comprising white petrolatum at a concentration of about 50% (w/w), mineral oil at a concentration of about 40% (w/w), and lanolin at a concentration of about 2% (w/w).

199. A method of treating a dermatological condition of a subject, comprising: a) providing the topical composition of any one of embodiments 161-198; b) topically applying a therapeutically effective amount of the topical composition to the subject's skin suspected of having a dermatological condition.

200. The method of embodiment 199, wherein the dermatological condition is excessive darkening of the subject's skin.

201. The method of embodiment 199, wherein the dermatological condition is a liver spot.

202. The method of embodiment 199, wherein the dermatological condition is hyperpigmentation.

203. The method of embodiment 199, wherein the dermatological condition is associated with overexposure to ultraviolet radiation.

204. The method of embodiment 199, wherein the dermatological condition is an age spot.

205. The method of embodiment 199, wherein the dermatological condition is seborrheic keratosis.

206. A method of lightening and/or whitening the skin color of a subject, comprising: a) providing the topical composition of any one of embodiments 161-198; and b) topically applying a therapeutically effective amount of the topical composition to the subject's skin.

207. A topical composition for treating acne, comprising a therapeutically effective amount of an acne agent and a therapeutically effective amount of at least one cannabinoid, its analogs, derivatives, or a combination thereof, wherein the cannabinoid, its analogs, derivatives, or a combination thereof is present in a concentration between about 0.001% to about 10% by weight of the composition, and appropriate topical pharmaceutically acceptable excipients or carriers.

208. The topical composition of embodiment 207, wherein the therapeutically effective amount of the cannabinoid, its analogs, derivatives, or a combination thereof is about 0.01% to about 4% by weight of the composition.

209. The topical composition of embodiment 207, wherein the therapeutically effective amount of the cannabinoid, its analogs, derivatives, or a combination thereof is about 0.1% to about 3% by weight of the composition.

210. The topical composition of any one of embodiments 207-209, the cannabinoid, its analogs, derivatives, or a combination thereof is one or more of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

211. The topical composition of any one of embodiments 207-210, wherein the acne agent is selected from the group consisting of salicylic acid, glycolic acid, or lactic acid.

212. The topical composition of any one of embodiments 207-211, wherein the acne agent is a retinol.

213. The topical composition of any one of embodiments 207-212, further comprising an antibiotic.

214. The topical composition of embodiment 213, wherein the antibiotic is selected from the group consisting of erythromycin, clindamycin, lymecycline, tinidazole, tetracycline, amoxicillin, ampicillin, lumefloxacin, norfloxacin, afloxam, ciproflaxin, azithromycin, and cefltoxine.

215. The topical composition of any one of embodiments 207-214, further comprising a steroid.

216. The topical composition of embodiment 215, wherein the steroid is selected from the group consisting of hydrocortisone, cortisone acetate, prednisone, prednisolone, methylprednisolone, dexamethasone, betamethasone, triamcinolone, and beclomethasone, fluorometholone. Other glucocorticoids useful in the treatment method include, but are not limited to, 21-acetoxypregnenolone, alelometasone, algestone, amcinonide, budesonide, chloroprednisone, clobetasol, clobetasone, clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, rimexolone, tixocortol, triamcinolone acetonide, triamcinolone benetonide, triamcinolone hexacetonide, their ophthalmically acceptable salts, combinations thereof, and mixtures thereof.

217. The topical composition of any one of embodiments 207-216, further comprising a vitamin.

218. The topical composition of embodiment 217, wherein the vitamin is selected from the group consisting of vitamin A, vitamin B, vitamin C, vitamin D, and vitamin E.

219. A method of treating acne of a subject, comprising: a) providing the topical composition of any one of embodiments 207-218; b) topically applying a therapeutically effective amount of the topical composition to the subject's skin suspected of having a dermatological condition.

220. A composition comprising a therapeutically effective amount of a cannabinoid, wherein the pharmaceutical composition is a powder comprising a guest particle adhered to a carrier particle.

221. The composition of embodiment 220, wherein the guest particle, carrier particle, or both comprise at least one cannabinoid.

222. The composition of any one of embodiments 220-221, wherein the cannabinoid is selected from the group consisting of cannabigerol-type, cannabichromene-type, a cannabidiol-type, a cannabinol-type, a cannabielsoin-type, and iso-tetrahydrocannabinol-type, a cannabicyclol-type, and a cannabicitran-type cannabinoid.

223. The composition of embodiment 222, wherein the cannabinoid is a cannabidiol-type cannabinoid.

224. The composition of embodiment 223, wherein the cannabidiol-type cannabinoid is selected from the group consisting of cannabidiol (CBD-1), cannabidiolic acid (CBDA), cannabidiol monomethyl ether (CBDM), cannabidiol-C4 (CBD-C4), cannabidivarinic acid (CBDVA), cannabidivarin (CBDV), and cannabidiorcol (CBD-C1).

225. The composition of any one of embodiments 220-224, wherein the guest particle, carrier particle, or both further comprise a bioactive agent.

226. The composition of embodiment 225, wherein the bioactive agent is a sedative, antianxiety agent, or a combination thereof.

227. The composition of embodiment 226, wherein the bioactive agent is selected from the group consisting of pentobarbital, secobarbital, diazepam, chlordiazepoxide, prazepam, clonazepam, midazolam, nitrazepam oxazepam, lorazepam, alprazolam, buspirone, flurazepam, temazepam, triazolam, chloral hydrate, zolpidem, zopiclone, eszopiclone, and diphenhydramine.

228. The composition of any one of embodiments 220-227, wherein the carrier particle, the guest particle, or both may comprise a solubility controlling agent, wherein the solubility controlling agent comprises an anionic surfactant, a cationic surfactant; a non-ionic surfactant, a zwitterionic surfactant, an amino acid, a sugar, a water-soluble polymer, a disintegrant, or a combination thereof.

229. The composition of any one of embodiments 220-228, wherein the carrier particle, the guest particle, or both comprises a terpene selected from the group consisting of myrcene, beta-caryophyllene, linalool, alpha-pinene, beta-pinene, ocimene, terpinolene, ocimene, terpinolene, alpha-terpineol, alpha-terpinene, gamma terpinene, alpha phellandrene, cymene, camphene, delta-3-carene, fenchol, 1,8-cineole, nerolidol, borneol, eucalyptol, camphene, and limonene, wherein the terpene is present at a concentration between about 0.01% to about 10% by weight of the composition.

230. The composition of any one of embodiments 220-229, wherein the guest particle is micronized particles, aggregated liposome, or aggregated nanoparticles.

231. The composition of embodiment 230, wherein the guest particle comprises least one cannabidiol.

232. The composition of any one of embodiments 220-231, wherein the carrier particle comprise lactose, D-mannitol, sorbitol, erythritol, α-trehalose dihydrate, dextrose, glucose monohydrate, maltitol, maltose, xylitol hydroxyapatite, D-raffinose anhydrous, raffinose pentahydrate, a surfactant, polyvinyl alcohol, polyvinylpyrrolidone, poly (lactic-co-glycolic acid) (PLGA)), microcrystalline cellulose (MCC), hydroxyl propyl methyl cellulose (HPMC), an amino acid, magnesium stearate or cyclodextrins.

233. The composition of any one of embodiments 220-231, wherein the pharmaceutical composition comprises two guest particle types adhered to one or more carrier particle types, wherein a first guest particle comprises a cannabinoid, and a second guest particle comprises a bioactive agent.

234. The composition of any one of embodiments 220-232, wherein the pharmaceutical composition comprises two or more carrier particle types adhered to one or more guest particle types.

235. The composition of embodiment 234, wherein the guest particle comprises least one cannabinoid.

236. The composition of any one of embodiments 220-235, wherein the guest particles are slowly released into a subject's lung when the pharmaceutical composition is inhaled by the individual.

237. The composition of any one of embodiments 220-236, wherein the carrier particle size is at least 5 times the average particle size of the guest particles.

238. The composition of any one of embodiments 220-237, wherein the pharmaceutical composition is formulated as a dry powder inhaler.

239. A method of treating a condition of a subject, comprising: a) providing the composition of any one of embodiments 220-238; and b) administering a therapeutically effective amount of the composition to the respiratory tract of the individual identified as having the condition; wherein the condition is selected from the group consisting of a sleep disorder, anxiety, post-traumatic stress disorder, a psychosomatic condition, a painful condition, an inflammatory condition, asthma, and diabetes.

240. The method of embodiment 239, wherein the composition is administered by insufflation.

241. The method of any one of embodiments 239-240, wherein the composition is administered to the nasal sinuses and/or the lungs.

242. The method of any one of embodiments 239-241, wherein the psychosomatic condition is insomnia.

243. A composition comprising a plurality of lipid-polymer composite particles encapsulating a bioactive agent, wherein the lipid-polymer composite particles comprise a block copolymer, a lipid selected from the group consisting of a neutral lipid, a cationic lipid, and an anionic lipid, and a sterol, wherein the plurality of lipid-polymer composite particles has a mean particle size of between 10 and 1000 nanometers.

244. The composition of embodiment 243, wherein the bioactive agent is a therapeutic agent, a nutraceutical agent, or a recreational agent.

245. The composition of embodiment 243, wherein the bioactive agent is a cannabinoid or a cannabinoid derivative, a terpene, a flavonoid, an antibiotic, an antiseptic agent, an antifungal, an antibacterial, an analgesic, an non-steroidal anti-inflammatory agent, an antiprotozoal agent, a steroid, an antiviral agent, a lipophilic drug, an anti-VEGF agent, an anti-glaucoma agent, nicotine or a nicotine analogue, cyclosporin A, tacrolimus, isotretinoin, propofol, griseofulvin or any combination thereof.

246. The composition of any one of embodiments 243-245, wherein the block copolymer is a poloxamer.

247. The composition of any one of embodiments 243-246, wherein the weight ratio of the poloxamer and the bioactive agent is between 2 and 15.

248. The composition of any one of embodiments 243-247, wherein the lipid comprises of a carbon chain of length from 4 to 22 and a neutral, cationic, or anionic head group.

249. The composition of embodiment 248, wherein the lipid is a phosphatidylcholine, a phosphatidylserine, a phosphatidylglycerol, a phosphatidylethanolamine, or a phosphatidylinositol.

250. The composition of any one of embodiments 243-249, wherein the concentration of the lipid is form about 0.1 mol % to about 10 mol %.

251. The composition of any one of embodiments 243-250, wherein the sterol is a phytosterol, or a synthetic sterol, or cholesterol, or a cholesterol analog.

252. The composition of any one of embodiments 243-251, wherein the concentration of the sterol is from about 5 mol % to about 50 mol % of the total lipid composition.

253. The composition of any one of embodiments 243-252, wherein the weight ratio of the sterol to the lipid is from about 0.01 to about 0.50.

254. A method of providing the bioactive agent to a subject, the method comprising administering to the subject a plurality of lipid-polymer composite particles encapsulating a bioactive agent, wherein the lipid-polymer composite particles comprise a polymer, a lipid and a sterol, wherein the plurality lipid-polymer composite particles has a mean particle size of between 10 nm and 1000 nanometers.

255. The method of embodiment 254, wherein the bioactive agent is a therapeutic agent, a nutraceutical agent, a recreational agent.

256. The method of embodiment 254, wherein the bioactive agent is a cannabinoid or a cannabinoid derivative, a terpene, a flavonoid, an antibiotic, an antiseptic agent, an antifungal, an antibacterial, an analgesic, a non-steroidal anti-inflammatory agent, an antiprotozoal agent, a steroid, an antiviral agent, a lipophilic drug, an anti-VEGF agent, an anti-glaucoma agent, nicotine or a nicotine analogue, cyclosporin A, tacrolimus, isotretinoin, propofol, griseofulvin or any combination thereof.

257. The method of any one of embodiments 254-256, wherein the dose of the cannabinoid is from about 0.01 mg/kg to about 30 mg/kg.

258. The method of any one of embodiments 254-257, wherein the block copolymer is a poloxamer.

259. The method of any one of embodiments 254-258, wherein the weight ratio between the poloxamer and the bioactive agent is from about 2 to about 15.

260. The method of any one of embodiments 254-259, wherein the lipid comprises of a carbon chain of length of from 4 to 22 and a neutral, cationic, or anionic head group.

261. The method of embodiment 260, wherein the lipid is a phosphatidylcholine, a phosphatidylserine, a phosphatidylglycerol, a phosphatidylethanolamine, or a phosphatidylinositol.

262. The method of any one of embodiments 254-261, wherein the concentration of lipid is between 0.1-10 mol %.

263. The method of any one of embodiments 254-262, wherein the sterol is a phytosterol, or a synthetic sterol, or cholesterol, or a cholesterol analog.

264. The method of any one of embodiments 254-263, wherein the concentration of the sterol is between 5-50 mol % of the total lipid composition.

265. The method of any one of embodiments 254-264, wherein the weight ratio of the sterol to the lipid is between 0.01 and 0.50.

266. The method of any one of embodiments 254-265, wherein the mode of administration is topically, orally, by injection, sublingually, buccally, rectally, vaginally, by ocular route, by otic route, by nasal route, by inhalation, by nebulization, ortransdermally.

267. A method of preparing a plurality of lipid-polymer composite particles encapsulating a bioactive agent, wherein the lipid-polymer composite particles comprise a block copolymer, a lipid selected from the group consisting of a neutral lipid, a cationic lipid, and an anionic lipid, and a sterol, wherein the plurality of lipid-polymer composite particles has a mean particle size of between 10 and 1000 nanometers, the method comprising homogenizing the bioactive agent with the polymer to produce a homogenized solution and injecting the lipid and the sterol into the homogenized solution.

268. The method of embodiment 267, wherein the bioactive agent is a therapeutic agent, a nutraceutical agent, or a recreational agent.

269. The method of any one of embodiments 267-268, wherein the bioactive agent is a cannabinoid or a cannabinoid derivative, a terpene, a flavonoid, an antibiotic, an antiseptic agent, an antifungal, an antibacterial, an analgesic, a non-steroidal anti-inflammatory agent, an antiprotozoal agent, a steroid, an antiviral agent, a lipophilic drug, an anti-VEGF agent, an anti-glaucoma agent, nicotine or a nicotine analogue, cyclosporin A, tacrolimus, isotretinoin, propofol, griseofulvin or any combination thereof.

270. The method of any one of embodiments 267-269, wherein the block copolymer is a poloxamer.

271. The method of any one of embodiments 267-270, wherein the weight ratio of the poloxamer and the bioactive agent is between 2 and 15.

272. The method of any one of embodiments 267-271, wherein the lipid comprises of a carbon chain of length from 4 to 22 and a neutral, cationic, or anionic head group.

273. The method of any one of embodiments 267-272, wherein the lipid is a phosphatidylcholine, a phosphatidylserine, a phosphatidylglycerol, a phosphatidylethanolamine, or a phosphatidylinositol.

274. The method of any one of embodiments 267-273, wherein the concentration of the lipid is form about 0.1 mol % to about 10 mol %.

275. The method of any one of embodiments 267-274, wherein the sterol is a phytosterol, or a synthetic sterol, or cholesterol, or a cholesterol analog.

276. The method of any one of embodiments 267-275, wherein the concentration of the sterol is from about 5 mol % to about 50 mol % of the total lipid composition.

277. The method of any one of embodiments 267-276, wherein the weight ratio of the sterol to the lipid is from about 0.01 to about 0.50.

278. The method of any one of embodiments 267-277, further comprising incorporating an antioxidant.

279. The method of embodiment 278, wherein the antioxidant is citric acid or sodium sulfite.

280. The method of any one of embodiments 267-279, further comprising incorporating the lipid via ethanol injection at a temperature between 25-75° C.

281. A composition comprising a therapeutically effective amount of a cannabinoid and micellar water.

282. The composition of embodiment 281, further comprising a therapeutically effective amount of a bioactive agent.

283. The composition of any one of embodiments 281-282, wherein the bioactive agent comprises an antibiotic, an antifungal, an antiviral, an analgesic, an antiseptic, a steroid, a non-steroidal anti-inflammatory drug,

284. The composition of embodiment 281, wherein the cannabinoid is present in a concentration between 0.01% to about 10% by weight of the composition.

285. The composition of any one of embodiments 281-284, wherein the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

286. A method of treating a dermatological condition of a subject, comprising: a) providing the composition of any one of embodiments 281-285; and b) administering a therapeutically effective amount of the composition to the skin of the subject identified as having the condition; wherein the condition is selected from the group consisting of acne, eczema, dermatitis, psoriasis, acne vulgaris, dryness of the skin, tinea pedis, hives, impetigo, non-melanoma cancer, itching dermatosis, acne rosacea, aging of skin, or dandruff.

287. A method of treating an ophthalmic condition of a subject, comprising: a) providing the composition of any one of embodiments 281-285; and b) administering a therapeutically effective amount of the composition to the eye of the subject identified as having the condition; wherein the condition is selected from the group consisting of dry eye, blepharitis, meibomianitis, keratitis, bacterial keratitis, protozoal keratitis, fungal keratitis, viral keratitis, conjunctivitis, bacterial conjunctivitis, viral conjunctivitis, episcleritis, scleritis, corneal abrasion, inflammation of the eye, injury to the eye following eye surgery, blepharoconjunctivitis, ocular rosacea, glaucoma, and contact lens intolerance.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A pharmaceutical composition for the treatment or prevention of an ophthalmic condition, comprising therapeutically effective amounts of at least one cannabinoid and a pharmaceutically acceptable excipient or carrier wherein the cannabinoid is present in a concentration between about 0.01% to about 10% by weight of the composition.

2. The pharmaceutical composition of claim 1, wherein the cannabinoid is selected from the group consisting of cannabidiolic acid (CBDA), cannabidiol (CBD-1), cannabidiol monomethyl ether (CBDM), cannabidiol-C4 (CBD-C4), cannabidivarinic acid (CBDVA), cannabidivarin (CBDV), and cannabidiorcol (CBD-C1).

3. The pharmaceutical composition of claim 1, further comprising:
(a) one or more terpenes selected from the group consisting of myrcene, beta-caryophyllene, linalool, alpha pinene, beta-pinene, ocimene, terpinolene, ocimene, terpinolene, alpha terpineol, alpha terpinene, gamma terpinene, alpha phellandrene, cymene, camphene, delta-3-carene, fenchol, 1,8-cineole, nerolidol, borneol, eucalyptol, camphene, and limonene, wherein one or more terpene is present at a concentration between about 0.01% to about 10% by weight of the composition;
(b) one or more flavonoid selected from the group consisting of cannaflavin A, cannaflavin B, phenolic acids, stilbenoids, phytochemicals, dihydroflavonols, anthocyanins, anthocyanidins, polyphenols, tannins, flavones, flavonols, flavan-3-ols, flavan-4-ol, flavan-3,4-diol, homoisoflavonoids, phenylpropanoids, phloroglucinols, coumarins, phenolic acids, naphthodianthrones, steroid glycosides, bioflavonoids, isoflavonoids, and a combination of one or more flavonoid, wherein one or more flavonoid is present at a concentration between about 0.01% to about 10% by weight of the composition;
(c) β-sitosterol, wherein the β-sitosterol is present at a concentration between about 0.01% to about 10% by weight of the composition;
(d) methyl salicylate, wherein the methyl salicylate is present at a concentration between about 0.01% to about 10% by weight of the composition;
(e) tocopherol, selected from the group consisting of alpha tocopherol, gamma tocopherol, and a combination of alpha and gamma tocopherol, wherein the tocopherol is present in a concentration between about 0.01% to about 10% by weight of the composition;
(f) alkaloids, lignans, or a combination of alkaloids and lignans;
(g) one or more active agents selected from the group consisting of an antibiotic, an antiseptic agent, an antifungal, an antibacterial agent, an analgesic, non-steroidal anti-inflammatory agent, antiprotozoal agent, steroid, and an antiviral agent; and/or
(h) a thickening agent, a solubilizing agent, a tonicity agent, a pH adiuster, a preservative, an antioxidant, osmotic agent, micellization agent, demulcent, surfactant, or a combination thereof.

4-10. (canceled)

11. The pharmaceutical composition of claim 1, wherein the composition is encapsulated in liposomes or is formulated as a gel, a thin film, an ointment, non-aqueous solution, a solid form, a paste, a liquid, an aerosol, a mist, a polymer, a film, an emulsion, a suspension, or an injectable formulation.

12. (canceled)

13. A pharmaceutical eluting contact lens comprising a contact lens coated or embedded with the pharmaceutical composition of claim 1, wherein the pharmaceutical composition is released from the contact lens when the lens is placed on an individual's eye.

14. A method of treating an ophthalmic condition, dry eye, meibomianitis, or blepharitis of an individual comprising
administering a therapeutically effective amount of the pharmaceutical composition of claim 1 to an eye of the individual.

15-17. (canceled)

18. A kit for treating an ophthalmic condition, comprising:
a) a first ophthalmic formulation comprising the pharmaceutical composition of claim 1 for administration during daytime; and
b) a second ophthalmic formulation containing a cannabinoid or derivatives thereof for administration prior to sleep,
wherein the second ophthalmic formulation has a higher viscosity than the first ophthalmic formulation.

19. (canceled)

20. A method for treating an ophthalmic condition of an individual, comprising:
a) administering a first ophthalmic formulation comprising the pharmaceutical composition of claim 1 to an eye of the individual, wherein the first ophthalmic formulation is a liquid ophthalmic formulation; and
b) administering a second ophthalmic formulation to an eye, wherein the second ophthalmic formulation comprises at least one cannabinoid or derivatives thereof in a concentration between about 0.01% to about 10% by weight of the composition, and a pharmaceutically acceptable excipient or carrier, and wherein the second ophthalmic formulation has a higher viscosity than the first ophthalmic formulation.

21. A pharmaceutical composition comprising therapeutically effective amounts of:
(a) a cannabinoid; and
(b) a parasympathetic agonist.

22-25. (canceled)

26. A method of treating a vision disorder comprising
administering a therapeutically effective amount of the pharmaceutical composition of claim 21 to an eye of a subject identified as having a vision disorder.

27. (canceled)

28. A topical composition for the treatment or prevention of a dermatological condition, comprising a therapeutically effective amount of at least one cannabidiol, its analogs, derivatives, or a combination thereof, wherein the cannabidiol, its analogs, derivatives, or a combination thereof is present in a concentration between about 0.001% to about 10% by weight of the composition, and appropriate topical pharmaceutically acceptable excipients or carriers.

29-31. (canceled)

32. A method of treating a dermatological condition lightening and/or whitening the skin color of a subject, or treating acne comprising
topically applying a therapeutically effective amount of the topical composition of claim 28 to the subject's skin.

33. (canceled)

34. A pharmaceutical composition for the treatment or prevention of bacterial vaginosis, comprising therapeutically effective amounts of at least one cannabinoid or derivatives thereof, and a pharmaceutically acceptable excipient or carrier, wherein the cannabinoid is present in a concentration between about 0.01% to about 30% by weight of the composition.

35. A method of treating an individual having bacterial vaginosis or candidiasis comprising
administering a therapeutically effective amount of the pharmaceutical composition of claim 34 to a vulvovaginal surface of the individual.

36-40. (canceled)

41. A kit for treating bacterial vaginosis or candidiasis, comprising:
a pessary containing or coated with the pharmaceutical composition of claim 34; and
a topical formulation comprising a cannabinoid or derivatives thereof.

42-50. (canceled)

51. A composition comprising a therapeutically effective amount of a cannabinoid, wherein the pharmaceutical composition is a powder comprising a guest particle adhered to a carrier particle.

52-55. (canceled)

56. A method of treating a condition of a subject, comprising:
administering a therapeutically effective amount of the composition of claim 51 to the respiratory tract of the individual identified as having the condition;
wherein the condition is selected from the group consisting of a sleep disorder, anxiety, post-traumatic stress disorder, a psychosomatic condition, a painful condition, an inflammatory condition, asthma, and diabetes.

57-72. (canceled)

73. A composition comprising a therapeutically effective amount of a cannabinoid and micellar water.

74. A method of treating a dermatological condition of a subject, comprising
administering a therapeutically effective amount of the composition of claim 73 to the skin of the subject identified as having the condition;
wherein the condition is selected from the group consisting of acne, eczema, dermatitis, psoriasis, acne vulgaris, dryness of the skin, tinea pedis, hives, impetigo, non-melanoma cancer, itching dermatosis, acne rosacea, aging of skin, or dandruff.

75. A method of treating an ophthalmic condition of a subject, comprising
administering a therapeutically effective amount of the composition of claim 73 to the eye of the subject identified as having the condition;
wherein the condition is selected from the group consisting of dry eye, blepharitis, meibomianitis, keratitis, bacterial keratitis, protozoal keratitis, fungal keratitis, viral keratitis, conjunctivitis, bacterial conjunctivitis, viral conjunctivitis, episcleritis, scleritis, corneal abrasion, inflammation of the eye, injury to the eye following eye surgery, blepharoconjunctivitis, ocular rosacea, glaucoma, and contact lens intolerance.
